SEQUENCE LISTING (17 sequences)

SEQ ID NO: 1 - protein, 266 aa, Deinococcus geothermalis
MQLDRYRVPPGQRVRLSNWPTDDDGGLSKAEGEALLPDLQQRLANLQERLYAESQQALLIVLQARDAGGKDGTVKHVIGAFNPSGVQVSNFKVPTEEERAHDFLWRIHRQTPRLGMIGVFNRSQYEDVLVTRVHHLIDDQTAQRRLKHICAFESLLTDSGTRIVKFYLHISPEEQKKRLEARLADPSKHWKFNPGDLQERAHWDAYTAVYEDVLTTSTPAAPWYVVPADRKWFRNLLVSQILVQTLEEMNPQFPAPAFNAADLRIV

SEQ ID NO: 2 - protein, 280 aa, Meiothermus ruber
MGFCSIEFLMGAQMKKYRVQPDGRFELKRFDPDDTSAFEGGKQAALEALAVLNRRLEKLQELLYAEGQHKVLVVLQAMDAGGKDGTIRVVFDGVNPSGVRVASFGVPTEQELARDYLWRVHQQVPRKGELVIFNRSHYEDVLVVRVKNLVPQQVWQKRYRHIREFERMLADEGTTILKFFLHISKDEQRQRLQERLDNPEKRWKFRMGDLEDRRLWDRYQEAYEAAIRETSTEYAPWYVIPANKNWYRNWLVSHILVETLEGLAMQYPQPETASEKIVIE

SEQ ID NO: 3 - protein, 268 aa, Meiothermus silvanus
MAKTIGATLNLQDIDPRSTPGFNGDKEKALALLEKLTARLDELQEQLYAEHQHRVLVILQGMDTSGKDGTIRHVFKNVDPLGVRVVAFKAPTPPELERDYLWRVHQHVPANGELVIFNRSHYEDVLVARVHNLVPPAIWSRRYDHINAFEKMLVDEGTTVLKFFLHISKEEQKKRLLERLVEADKHWKFDPQDLVERGYWEDYMEAYQDVLDKTHTQYAPWHVIPADRKWYRNLQVSRLLVEALEGLRMKYPRPKLNIPRLKSELEKM

SEQ ID NO: 4 - protein, 300 aa, Thermosynechococcus elongatus
MIPQDFLDEINPDRYIVPAGGNFHWKDYDPGDTAGLKSKVEAQELLAAGIKKLAAYQDVLYAQNIYGLLIIFQAMDAAGKDSTIKHVMSGLNPQACRVYSFKAPSAEELDHDFLWRANRALPERGCIGIFNRSYYEEVLVVRVHPDLLNRQQLPPETKTKHIWKERFEDINHYERYLTRNGILILKFFLHISKAEQKKRFLERISRPEKNWKFSIEDVRDRAHWDDYQQAYADVFRHTSTKWAPWHIIPANHKWFARLMVAHFIYQKLASLNLHYPMLSEAHREQLLEAKALLENEPDED

SEQ ID NO: 5 - protein, 281 aa, Anaerolinea thermophila
MGEAMERYFIKPGEKVRLKDWSPDPPKDFEGDKESTRAAVAELNRKLEVLQERLYAERKHKVLVILQGMDTSGKDGVIRSVFEGVNPQGVKVANFKVPTQEELDHDYLWRVHKVVPGKGEIVIFNRSHYEDVLVVRVHNLVPPEVWKKRYEQINQFERLLHETGTTILKFFLFISREEQKQRLLERLADPAKHWKFNPGDLKERALWEEYEKAYEDVLSRTSTEYAPWILVPADKKWYRDWVISRVLVETLEGLEIQLPPPLADAETYRRQLLEEDAPESR

SEQ ID NO: 6 - protein, 270 aa, Caldilinea aerophila
MDVDRYRVPPGSTIHLSQWPPDDRSLYEGDKKQGKQDLSALNRRLETLQELLYAEGKHKVLIILQGMDTSGKDGVIRHVFNGVNPQGVKVASFKVPTAVELAHDFLWRIHRQTPGSGEIVIFNRSHYEDVLVVRVHGLVPPEVWARRYEHINAFEKLLVDEGTTILKFFLHISKEEQRQRLLERLEMPEKRWKFSVGDLAERKRWDEYMAAYEAVLSKTSTEYAPWYIVPSDRKWYRNLVISHVIINALEGLNMRYPQPEDIAFDTIVIE

SEQ ID NO: 7 - protein, 294 aa, Chlorobaculum tepidum
MKLDLDAFRIQPGKKPNLAKRPTRIDPVYRSKGEYHELLANHVAELSKLQNVLYADNRYAILLIFQAMDAAGKDSAIKHVMSGVNPQGCQVYSFKHPSATELEHDFLWRTNCVLPERGRIGIFNRSYYEEVLVVRVHPEILEMQNIPHNLAHNGKVWDHRYRSIVSHEQHLHCNGTRIVKFYLHLSKEEQRKRFLERIDDPNKNWKFSTADLEERKFWDQYMEAYESCLQETSTKDSPWFAVPADDKKNARLIVSRIVLDTLESLNLKYPEPSPERRKELLDIRKRLENPENGK

SEQ ID NO: 8 - protein, 269 aa, Oceanithermus profundus
MDVSRYRVPPGSGFDPEAWPTREDDDFAGGKKEAKKELARLAVRLGELQARLYAEGRQALLIVLQGMDTAGKDGTIRHVFRAVNPQGVRVTSFKKPTALELAHDYLWRVHRHAPARGEIGIFNRSHYEDVLVVRVHELVPPEVWGRRYDHINAFERLLADEGTRIVKFFLHISKDEQKRRLEARLENPRKHWKFNPADLSERARWGDYAAAYAEALSRTSSDRAPWYAVPADRKWQRNRIVAQVLVDALEAMDPRFPRVDFDPASVRVE

SEQ ID NO: 9 - protein, 290 aa, Roseiflexus castenholzii
MYAQRVVPGMRVRLHDIDPDANGGLNKDEGRARFAELNAELDVMQEELYAAGIHALLLILQGMDTAGKDGAIRNVMLNLNPQGCRVESFKVPTEEELAHDFLWRVHRVVPRKGMVGVFNRSHYEDVLVVRVHSLVPESVWRARYDQINAFERLLADTGTIIVKCFLHISKEEQEQRLLARERDVSKAWKLSAGDWRERAFWDDYMAAYEEALTRCSTDYAPWYIIPANRKWYRDLAISEALVETLRPYRDDWRRALDAMSRARRAELEAFRAEQHAMEGRPQGAGGVSRR

SEQ ID NO: 10 - protein, 282 aa, Roseiflexus sp
MHYAHTVIPGTQVRLRDIDPDASGGLTKDEGRERFASFNATLDAMQEELYAAGVHALLLILQGMDTAGKDGAIRNVMHNLNPQGCRVESFKVPTEEELAHDFLWRVHKVVPRKGMVGVFNRSHYEDVLVVRVHSLVPEHVWRARYDQINAFERLLTDTGTIIVKCFLHISKDEQEKRLLAREQDVTKAWKLSAGDWRERERWDEYMAAYEEALTRCSTEYAPWYIIPANRKWYRDLAISEVLVETLRPYRDDWQRALDAMSQARLAELKAFRHQQTAGATRL

SEQ ID NO: 11 - protein, 274 aa, Truepera radiovictrix
MSQGSAKGLGKLDKKVYARELALLQLELVKLQGWIKAQGLKVVVLFEGRDAAGKGSTITRITQPLNPRVCRVVALGAPTERERTQWYFQRYVHHLPAAGEMVLFDRSWYNRAGVERVMGFCTEAEYREFLHACPTFERLLLDAGIILIKYWFSVSAAEQERRMRRRNENPAKRWKLSPMDLEARARWVAYSKAKDAMFYHTDTKASPWYVVNAEDKRRAHLSCIAHLLSLIPYEDLTPPPLEMPPRDLAGADEGYERPDKAHQTWVPDYVPPTR

SEQ ID NO: 12 - protein, 186 aa, Thermus thermophilus
MDVGQAVIFLGPPGAGKGTQASRLAQELGFKKLSTGDILRDHVARGTPLGERVRPIMERGDLVPDDLILELIREELAERVIFDGFPRTLAQAEALDRLLSETGTRLLGVVLVEVPEEELVRRILRRAELEGRSDDNEETVRRRLEVYREKTEPLVGYYEARGVLKRVDGLGTPDEVYARIRAALGI

SEQ ID NO: 13 - protein, 208 aa, Thermus thermophilus
MRGIVTIDGPSASGKSSVARRVAAALGVPYLSSGLLYRAAAFLALRAGVDPGDEEGLLALLEGLGVRLLAQAEGNRVLADGEDLTSFLHTPEVDRVVSAVARLPGVRAWVNRRLKEVPPPFVAEGRDMGTAVFPEAAHKFYLTASPEVRAWRRARERPQAYEEVLRDLLRRDERDKAQSAPAPDALVLDTGGMTLDEVVAWVLAHIRR

SEQ ID NO: 14 - protein, 225 aa, Pyrococcus furiosus
MRIVFDIGGSVLVPENPDIDFIKEIAYQLTKVSEDHEVAVVVGGGKLARKYIEVAEKFNSSETFKDFIGIQITRANAMLLIAALREKAYPVVVEDFWEAWKAVQLKKIPVMGGTHPGHTTDAVAALLAEFLKADLLVVITNVDGVYTADPKKDPTAKKIKKMKPEELLEIVGKGIEKAGSSSVIDPLAAKIIARSGIKTIVIGKEDAKDLFRVIKGDHNGTTIEP

SEQ ID NO: 15 - protein, 207 aa, Thermotoga maritima [sequence omitted]

SEQ ID NO: 16 - protein, 142 aa, Aquifex aeolicus
MAVERTLIIVKPDAMEKGALGKILDRFIQEGFQIKALKMFRFTPEKAGEFYYVHRERPFFQELVEFMSSGPVVAAVLEGEDAIKRVREIIGPTDSEEARKVAPNSIRAQFGTDKGKNAIHASDSPESAQYEICFIFSGLEIV

SEQ ID NO: 17 - DNA, 15 bp, Unknown
  Initially Transcribed Sequence
gggagaccaggaatt

(19) United States
(12) Patent Application Publication
(10) Pub. No.: US 2022/0162659 A1
(43) Pub. Date: May 26, 2022

(54) CELL-FREE PRODUCTION OF RIBONUCLEIC ACID

(71) Applicants: Andrey J. ZARUR, Winchester, MA (US); Drew S. CUNNINGHAM, Winchester, MA (US); James R. ABSHIRE, Cambridge, MA (US); Rachit JAIN, Medford, MA (US); Michael E. HUDSON, Framingham, MA (US); GREENLIGHT BIOSCIENCES, INC., Medford, MA (US)

(72) Inventors: Andrey J. ZARUR, Winchester, MA (US); Drew S. CUNNINGHAM, Winchester, MA (US); James R. ABSHIRE, Cambridge, MA (US); Rachit JAIN, Medford, MA (US); Michael E. HUDSON, Framingham, MA (US)

(21) Appl. No.: 17/441,448

(22) PCT Filed: Mar. 30, 2020
(86) PCT No.: PCT/US2020/025824

§ 371 Date: Sep. 21, 2021

Related U.S. Application Data
(60) Provisional application No. 62/826,983, filed on Mar. 29, 2019.

(51) Int. Cl. C12P 19/34; C12N 9/12; C12N 9/22; C12N 1/06; C12Q 1/6844

(52) U.S. Cl. CPC C12P 19/34; C12Y 301/30001; C12N 9/1247; C12N 9/1258; C12N 9/22; C12N 1/06; C12Q 1/6844; C12Y 207/04001; C12Y 207/04003; C12Y 207/04006; C12Y 207/04008; C12Y 207/04022; C12Y 207/04025; C12Y 207/07006; C12Y 207/07008; C12Y 301/13001; C12N 9/1229

(57) ABSTRACT

This invention relates to in vitro production of nucleic acids, particularly RNAs and specifically messenger RNAs (mRNA).

### RELATED APPLICATION

This application claims priority to U.S. Provisional Patent Application No. 62/826,983 filed Mar. 29, 2019, which is hereby incorporated in its entirety.

This invention relates to methods disclosed in International Application No. PCT/US2018/05535 filed Oct. 11, 2018, entitled “Methods and Compositions for Nucleoside Triphosphate and Ribonucleic Acid Production,” incorporated by reference in its entirety herein.

### FIELD OF THE INVENTION

This invention relates to in vitro production of nucleic acids, particularly RNAs and specifically messenger RNAs (mRNA), and more specifically eukaryotic mRNAs. The reagents and methods disclosed herein enable in vitro production of mRNA at low cost, high efficiency, and at commercially useful scale.

### BACKGROUND OF RELATED ART

Ribonucleic acid (RNA) is ubiquitous to life, acting as the key messenger of information in cells, carrying the instructions from DNA for the regulation and synthesis of proteins. RNA is of interest in biotechnology as synthetically modulating mRNA levels in cells has applications in fields such as agricultural crop protection, anti-cancer therapeutics, gene therapies, vaccines, immune system modulation, disease detection, and animal health. Functional single-stranded (e.g. mRNA) and double-stranded RNA molecules have been produced in living cells and in vitro using purified, recombinant enzymes and purified nucleotide triphosphates (see, e.g., European Patent No. 1631675, U.S. Patent Application Publication No. 2014/0271559 A1, and PCT/US2018/05535, each of which is incorporated herein by reference). Nonetheless, the production of RNA and specifically mRNA at scales enabling widespread commercial application is currently cost-prohibitive. Methods are needed that are cheaper; faster; and easily executed, preferably without the need for external suppliers of specialty reagents as a means of providing firmer control of the process to benefit product safety and quality; and that generate RNA, specifically mRNA, of comparable quantity and grade as prior art methods.

### SUMMARY OF THE INVENTION

Provided herein are reagents and methods for producing in vitro RNA, particularly mRNA, in commercially useful quantities and costs.

As described below, in one aspect, the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; and (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, vii) at least one DNA template encoding an mRNA with a polyA tail, and viii) one or more capping reagents are added under conditions that produce mRNA, and further wherein, optionally ix) at least one deoxyribonuclease is added under conditions that digest the DNA following RNA production.

In a second aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, vii) at least one DNA template encoding an mRNA, and viii) one or more capping reagents are added under conditions that produce capped RNA and further wherein optionally ix) at least one deoxyribonuclease is added under conditions that digest the DNA following RNA production; and d) (i) further incubating said reaction mixture produced in step (c) in the presence of a polyA polymerase and ATP, under conditions that produce mRNA or (ii) removing the RNA polymerase from the reaction mixture of step (c) by inactivation or physical separation followed by producing mRNA by further incubating the reaction mixture in the presence of polyA polymerase and ATP.

In a third aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase vii) at least one DNA template encoding an mRNA with a poly A tail, are added under conditions that produce uncapped RNA, and further wherein optionally viii) at least one deoxyribonuclease is added under conditions that digest the DNA following RNA production; and (d) exchanging buffer conditions from said reaction mixture from step (c) and incubating said reaction mixture in the presence of capping enzymes, GTP, and a methyl donor to produce mRNA.

In a fourth aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, and vii) at least one DNA template encoding an mRNA are added under conditions that produce uncapped, untailed RNA, and further wherein, optionally viii) at least one deoxyribonuclease is added under conditions that digest the DNA following RNA production; d) (i) further incubating said reaction mixture produced in step (c) in the presence of a polyA polymerase and ATP, under conditions that produce uncapped RNA; or (ii) removing the RNA polymerase from the reaction mixture of step (c) by inactivation or physical separation followed by producing uncapped RNA by further incubating the reaction mixture in the presence of polyA polymerase and ATP; and e) exchanging buffer conditions from said reaction mixture from step (c) and incubating said reaction mixture in the presence of capping enzymes, GTP, and a methyl donor to produce mRNA.

In a fifth aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; and (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase, vii) at least one DNA template encoding an mRNA with a polyA tail, and viii) one or more capping reagents are added under conditions that produce mRNA, and further wherein optionally ix) at least one deoxyribonuclease is added under conditions that digest the DNA following RNA production.

In a sixth aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase, vii) at least one DNA template encoding an mRNA, and viii) one or more capping reagents are added under conditions that produce capped RNA and further wherein optionally ix) at least one deoxyribonuclease is added under conditions that digest the DNA following RNA production; and d) (i) further incubating said reaction mixture produced in step (c) in the presence of a polyA polymerase and ATP, under conditions that produce mRNA or (ii) removing the RNA polymerase from the reaction mixture of step (c) by inactivation or physical separation followed by producing mRNA by further incubating the reaction mixture in the presence of polyA polymerase and ATP.

In a seventh aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase and vii) at least one DNA template encoding an mRNA with a poly A tail are added under conditions that produce uncapped RNA; and further wherein optionally viii) at least one deoxyribonuclease is added under conditions that digest the DNA following RNA production; and (d) exchanging buffer conditions from said reaction mixture from step (c) and incubating said reaction mixture in the presence of capping enzymes, GTP, and a methyl donor to produce mRNA.

In an eighth aspect the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase and vii) at least one DNA template encoding an mRNA, are added under conditions that produce uncapped, untailed RNA, and further wherein optionally viii) at least one deoxyribonuclease is added under conditions that digest the DNA following RNA production; (d) further incubating said reaction mixture produced in step (c) in the presence of a polyA polymerase and ATP, under conditions that produce uncapped RNA; or (ii) removing the RNA polymerase from the reaction mixture of step (c) by inactivation or physical separation followed by producing uncapped RNA by further incubating the reaction mixture in the presence of polyA polymerase and ATP; and e) exchanging buffer conditions from said reaction mixture from step (c) and incubating said reaction mixture in the presence of capping enzymes, GTP, and a methyl donor to produce mRNA.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1. Schematic of method to produce mRNA. 1A) PolyA Tail Encoded in DNA Template & Capping via Capping Reagent; B) Enzymatic Addition of PolyA Tail & Capping via Capping Reagent; C) PolyA Tail Encoded in DNA Template & Capping via Capping Enzymes; and D) Enzymatic Addition of PolyA Tail & Capping via Capping Enzymes.

FIG. 2. Biosynthetic pathway for production of RNA. A biosynthetic pathway for producing NTPs, and downstream RNA, using cellular RNA as the starting material is shown. In this pathway, a ribonuclease is used to degrade cellular RNA into NMPs or NDPs (FIG. 2b).

FIG. 3. RNA products 1. An agarose gel of RNA products produced in reactions comprising RNA polymerase and NMPs produced by depolymerization (− NMPs) or purified NMPs (+ NMPs, 4 mM each) is shown. Abbreviations: −2 log: 2-log DNA ladder (New England Biolabs), NMPs: equimolar mix of 5′-NMPs, RNA Pol: thermostable T7 RNA polymerase, Template 1: Linear DNA template, Template 2: Plasmid DNA template.

FIG. 4. RNA Products 2. An agarose gel of RNA products produced in reactions comprising RNA polymerase and NMPs produced by depolymerization of purified RNA is shown. As a negative control, a reaction was performed in the absence of RNA polymerase. Abbreviations: −2 log: 2-log DNA ladder (New England Biolabs), NMPs: equimolar mixture of 5′-nucleoside monophosphates, RNA Pol: thermostable T7 RNA polymerase, Template 1: Linear DNA template, Template 2: Plasmid DNA template.

FIG. 5. RNA Products 3. An agarose gel of RNA products produced by cell-free RNA (CFR) synthesis using a wild-type polymerase (W) or a thermostable polymerase mutant (T) at 37° C. is shown. Abbreviations: −2 log: 2-log DNA ladder (New England Biolabs), W: wild-type T7 RNA polymerase (New England Biolabs), T: thermostable T7 RNA polymerase, Template 1: Linear DNA template, Template 2: Plasmid DNA template.

FIG. 6. Nucleotides produced over time. A graph of acid-soluble nucleotides (mM) produced over time during depolymerization of various sources of RNA using purified RNase R or Nuclease P1. Acid-soluble nucleotides were measured by UV absorbance.

FIG. 7. Available NMPs produced by Nuclease P1. A graph of the percent of available 5′-NMPs produced over time during depolymerization of RNA from E. coli or yeast using Nuclease P1 is shown. Percent of available 5′-NMPs was determined by liquid chromatography-mass spectrometry (LC-MS).

FIG. 8. Analysis of different lysate temperatures. Nucleomic profile plots for RNA depolymerization across different temperatures of a lysate from E. coli. Cumulative concentrations of 20 analytes are shown. Nucleosides are shown in a white-speckled pattern, and were minimally produced. Data for 50° C. was also collected but is not shown.

FIG. 9. Analysis of cell-free synthesis of NTPs. A graph showing that cell-free synthesis of NTPs results in similar NTP titers regardless of nucleotide source after a 1 hour incubation at 48° C. is presented. For each source of nucleotides (cellular RNA, purified NMPs, or purified NDPs), a quantity of substrate sufficient to provide approximately 4 mM of each nucleotide was added to the reaction. For example, reactions with NDPs comprised 4 mM each ADP, CDP, GDP, and UDP.

FIG. 10. Expression of green fluorescent protein (GFP) in CFR mRNA-transfected HeLa cells. mRNA produced by in vitro transcription is presented as a control. Fluorescence microscopy images are shown.

FIG. 11. Quantification of GFP expression resulting from mRNAs with differing untranslated regions (UTRs). Relative fluorescence units (RFUs) are shown. UTR source genes: HSD, 5′ hydroxysterol dehydrogenase, 3′ albumin; COX, 5′ cytochrome oxidase, 3′ albumin; HBG, 5′, 3′ human β-globin; XBG, 5′, 3′ Xenopus β-globin.

FIG. 12. Capillary gel electrophoresis analysis of mRNA. Capillary gel electrophoresis results are shown for mRNA produced by in vitro transcription (IVT) and CFR, along with the percentage in each sample of total nucleic acid representing the mRNA species of interest.

FIG. 13. mRNA in CFR or IVT-produced RNA. An immunoblot is shown. Ref, reference mRNA available commercially.

FIG. 14. Endotoxin analysis of mRNA preparations. Endotoxin units (EU) per mL are shown.

FIG. 15. Yield and composition of mRNAs after reversed-phase ion-pair high performance liquid chromatography. A chromatogram and analysis of percent by mass of nucleic acid, protein, salts, unreacted NMPs, and other dry solids is shown for the samples, along with the percentage of nucleic acid representing the mRNA species of interest (top) and the overall purity (% nucleic acid in sample×% nucleic acid that is species of interest, bottom).

FIG. 16. Enzyme-linked Immunosorbant Assay (ELISA)-quantified production of Hemagglutinin (HA) in HeLa extracts using CFR mRNA. Concentration is shown in ng/mL. HBG (5′, 3′ human β-globin) and XBG (5′, 3′ Xenopus β-globin) represent different UTRs. Both crude and HPLC-purified samples are shown.

FIG. 17. Western blot of production of HA in HeLa extracts using CFR mRNA. A comparison to production from mRNA produced through in vitro transcription is shown. The arrow highlights the expected size of the HA protein (63 kDa).

FIG. 18. Luminescence quantification of firefly luciferase expression in HeLa extracts using CFR mRNA. HBG (5′, 3′ human β-globin) and XBG (5′, 3′ Xenopus β-globin) represent different UTRs. -mRNA, no RNA (negative control).

FIG. 19. Luminescence quantification of firefly luciferase expression in HeLa cells using CFR mRNA. HBG(1) and HBG(2) represent high and low levels of lipofectamine, as indicated. Multiple time points are shown.

FIG. 20. In vivo Imaging System (IVIS) images of luciferase expression in mice using CFR mRNA. LNP, lipid nanoparticles. GenVoy, commercial formulation. Arrows highlight areas of luminescence indicating expression of luciferase.

FIG. 21. Luminescence quantification of luciferase expression in vivo using CFR mRNA. Measurements from 0 to 72 hours after administration are shown against results from IVT mRNA (see labels).

FIG. 22. Nucleoside-modified mRNAs with ARCA capping. (A) Purity of CFR-produced mRNA versus IVT produced mRNA; (B) Quantification of nucleoside modification and capping (GLB—GreenLight Bio; IVT—in vitro transcription).

FIG. 23. Target gene expression in mice. Target gene expression at 5, 24, 48, and 72 hours after injection of D-luciferin. Gene expression was achieved by formulating mRNA proprietary lipid nanoparticle formulations into BALB/c mice (n=10/group, IM) at 0.2 μg dose (left) and 1 μg dose.

FIG. 24. Serum immunity in mice. Titers from mice treated with 30 g and 3 g doses of hemagglutinin mRNA versus positive control mice treated with inactivated H1N1. Circles indicate mice selected for subsequent challenge study.

FIG. 25. Body weight changes. Mice administered HA mRNA (30 μg) or inactivated H1N1 were protected from influenza-associated weight loss while untreated mice (circles) and mice treated with FLuc mRNA (30 μg; squares) showed decreases in body weight.

FIG. 26. RNA production. (A) Electropherograms of uncapped IVT-produced mRNA (reference); (B) electropherogram of CFR-produced mRNA using cellular RNA-derived nucleotides; and (C) electropherogram of uncapped CFR-produced mRNA using an equimolar mix of purified nucleoside monophosphates (5 mM each).

FIG. 27. CFR-produced mRNAs and polyA tail length. Overlay electropherogram of CFR-produced mRNAs with polyA tails of 0, 50, 100, or 150 nucleotides in length.

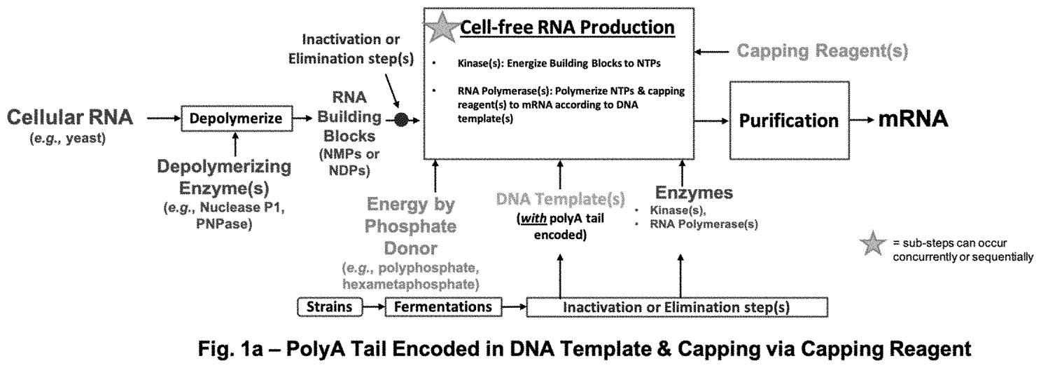

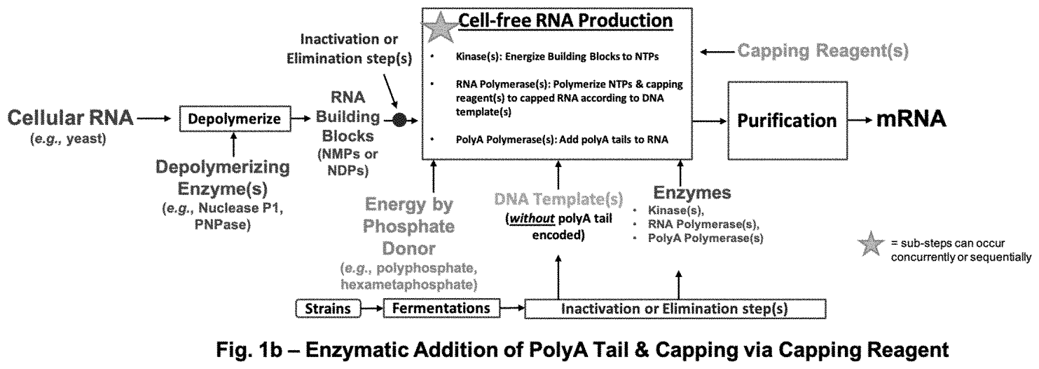

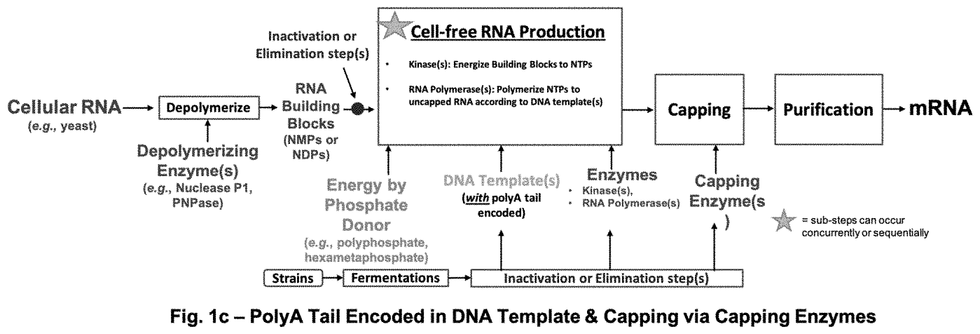

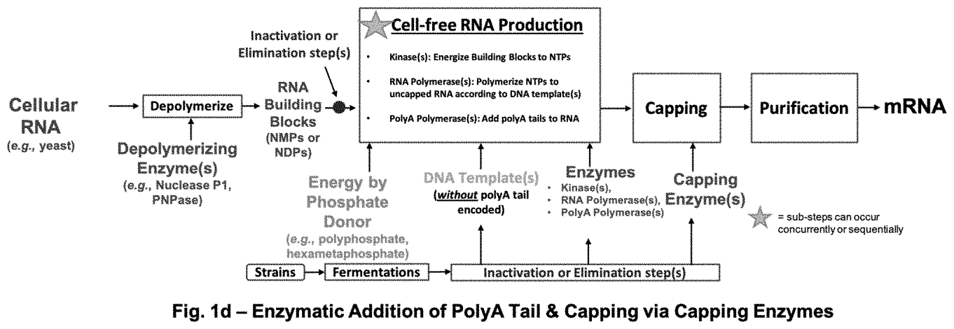

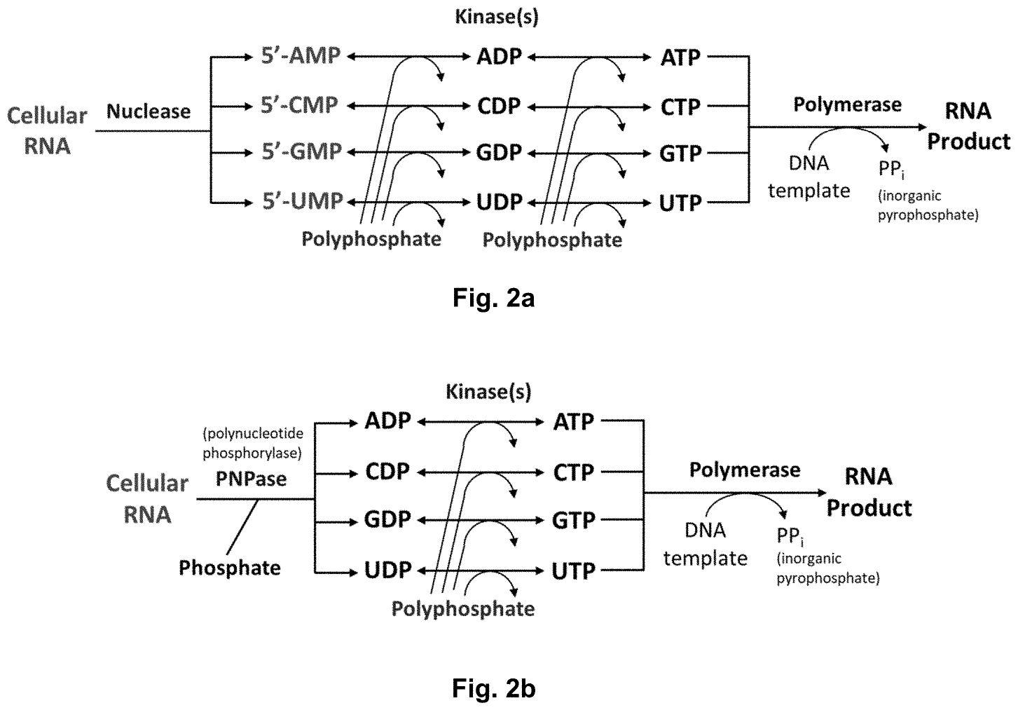

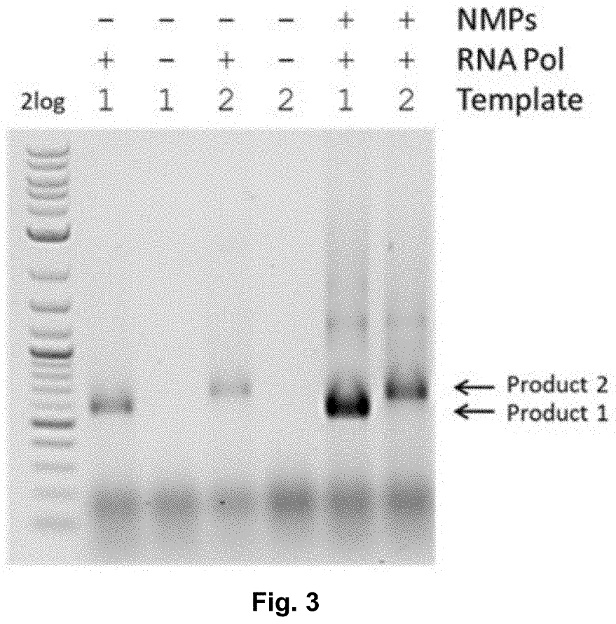

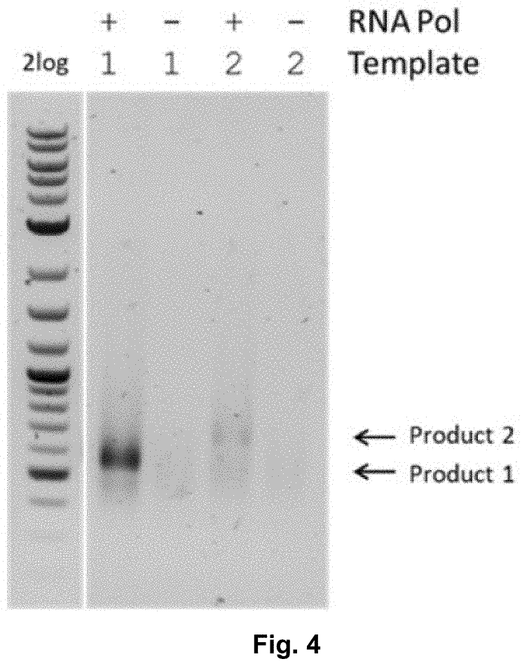

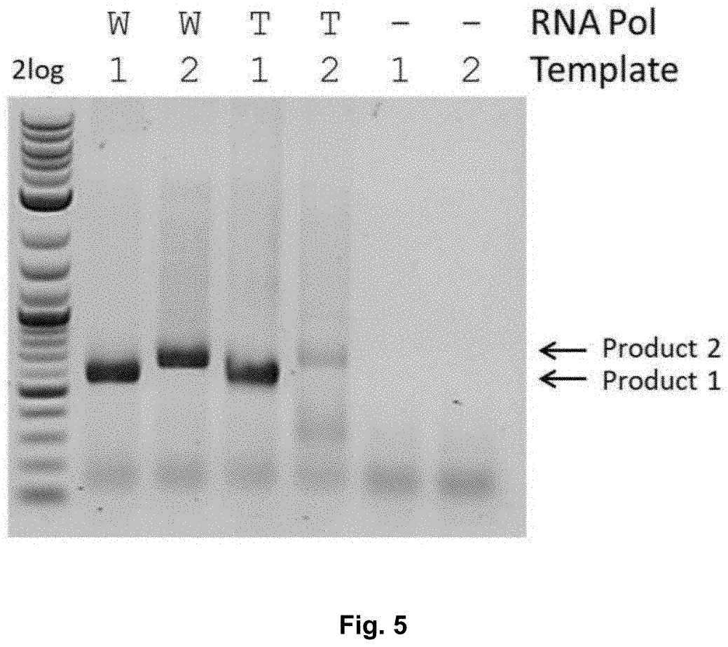

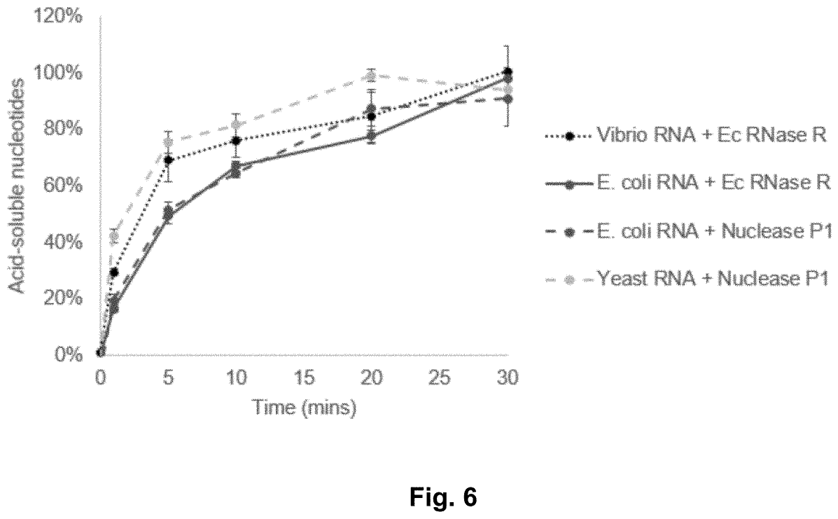

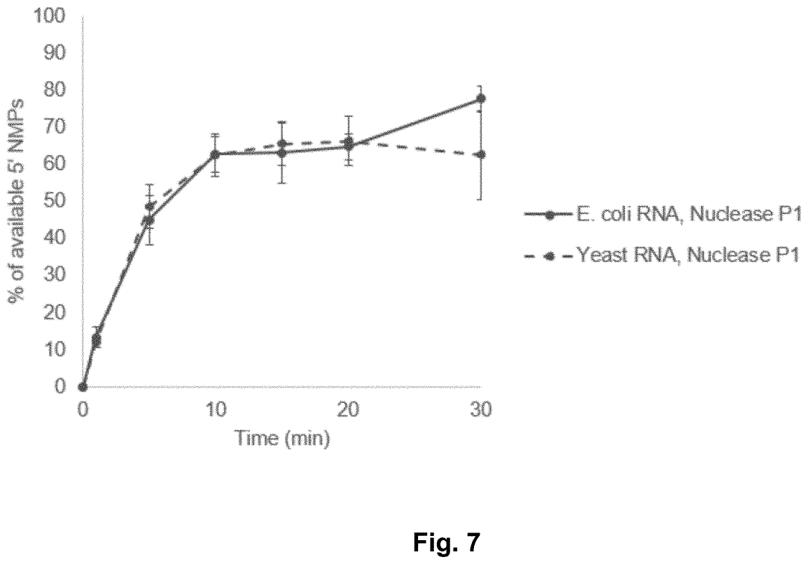

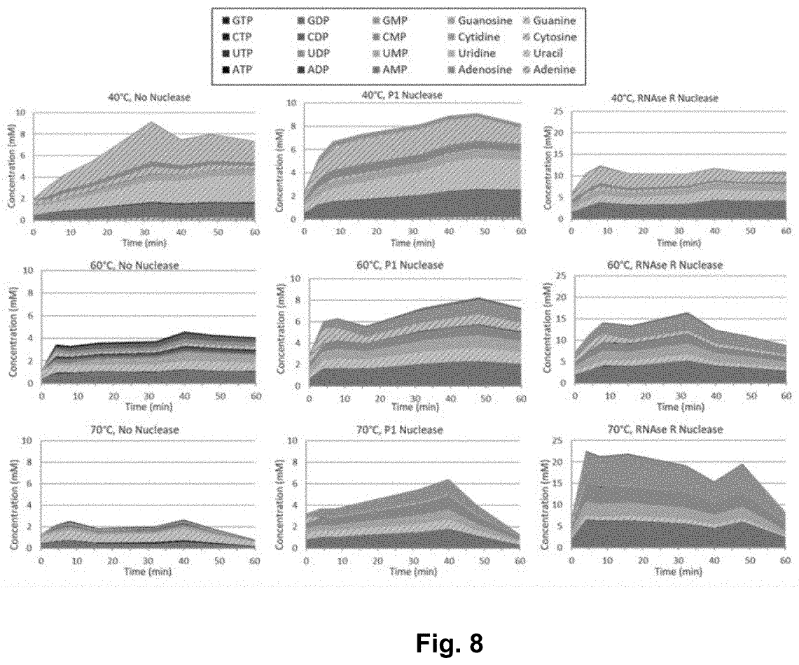

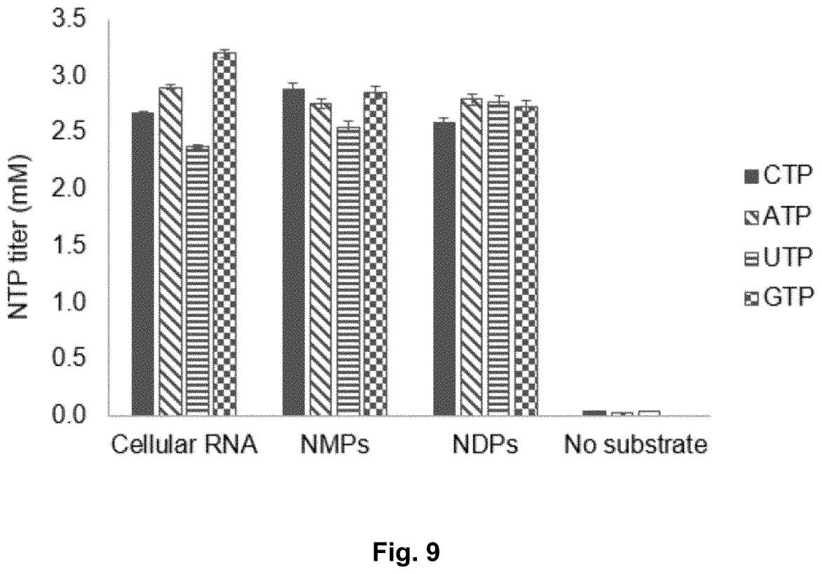

### DETAILED DESCRIPTION OF PREFERRED EMBODIMENTS

Provided herein are methods, compositions, cells, constructs, and systems for cell-free production (biosynthesis) of RNA and specifically messenger RNA (mRNA), and more specifically eukaryotic mRNA. In certain embodiments as set forth with further specificity below, this disclosure provides methods for cell-free RNA (CFR) production using inexpensive, scalable starting material (or “biomass”) to supply the building blocks for RNA. In certain embodiments as set forth with further specificity below, the methods provided herein comprise the following generally described steps of the disclosed methods.

### Conversion of Cellular RNA to Nucleoside Monophosphates

To produce nucleoside monophosphate “building blocks” of RNA, cellular (endogenous) RNA is incubated in a cell-free reaction mixture with one or more enzymes that depolymerize cellular RNA (comprising, inter alia, ribosomal or rRNA; messenger or mRNA; and transfer RNA or tRNA) into its constituent 5′ nucleoside monophosphates (NMPs). In certain embodiments, the RNA depolymerizing enzyme is a ribonuclease (e.g., Nuclease P1, RNase R) that depolymerizes the cellular RNA to 5′-NMPs. Cells, as the source of RNA, can be engineered to express the nuclease, or the nuclease can be produced by a separate cell and introduced to the reaction. For example, cellular RNA from yeast might be depolymerized by Nuclease P1 that was produced by Penicillium citrinum. Nuclease P1 is a zinc-dependent single-nuclease that hydrolyzes single-stranded RNA and DNA to RNA into 5′ nucleoside monophosphates. The enzyme has no base specificity.

Thereafter (i.e., when the cellular RNA has been depolymerized), the nuclease is eliminated (e.g. by physical separation, such as filtration, precipitation, capture, and/or chromatography) or inactivated (e.g. by temperature, pH, salt, detergent, alcohol or other solvents, and/or chemical inhibitors) in some embodiments. In order to contrast the RNA synthesis methods described herein to the RNA synthesis method of in vitro transcription (IVT), RNA synthesis methods described herein are denoted as “cell-free” RNA synthesis. While the IVT method is also technically cell-free, this method relies on direct addition of nucleotide substrates that are triphosphorylated and, therefore, does not require additional energy. The term “cell-free” is used for contrast, to denote RNA synthesis methods that allow for nucleotide substrates that need not be triphosphorylated (e.g., 5′-nucleoside monophosphates and/or nucleoside diphosphates), as the methods provide for a kinase enzyme (or enzymes) and an energy source (e.g., a phosphate donor like polyphosphate or hexametaphosphate) to convert nucleotides of a lower degree of phosphorylation to the their respective triphosphorylated forms.

### Conversion of Cellular RNA to Nucleoside Diphosphates

In other embodiments, the cellular RNA is depolymerized into nucleoside diphosphates (NDPs). In alternative embodiments, the RNA depolymerizing enzyme is a ribonuclease (e.g. polynucleotide phosphorylase (PNPase)) that depolymerizes the cellular RNA to NDPs in the presence of phosphate. Cells, as the source of RNA, can be engineered to express the cell-specific nuclease, or the nuclease can be produced by a separate cell and introduced to the reaction. Thereafter (i.e., when the cellular RNA has been depolymerized), the nuclease is eliminated (e.g. by physical separation, such as filtration, precipitation, capture, and/or chromatography) or inactivated (e.g. by temperature, pH, salt, detergent, alcohol or other solvents, and/or chemical inhibitors) in some embodiments.

### Production of Nucleoside Triphosphates

After depolymerization, the cell-free reaction mixture is incubated under conditions that result in phosphorylation of the NMPs or NDPs to NTPs (nucleoside triphosphates) using a plurality or mixture of kinase enzymes, including nucleoside monophosphate kinases which in some embodiments are specific for phosphorylating each of the individual NMPs in the mixture (i.e., AMP, GMP, CMP and UMP), a nucleoside diphosphate kinase (NDK), and a polyphosphate kinase (PPK). In the case where the depolymerization reaction produces NDPs (e.g. when using PNPase), the mixture would consist of a nucleoside diphosphate kinase (NDK), a polyphosphate kinase (PPK), and optionally, one or more nucleoside monophosphate kinases to salvage any NMPs generated through reversible reactions with NDK and PPK. Kinases can be produced at high titer in fermentations (e.g., in E. coli cells). The cells can then be lysed (e.g. using high-pressure homogenization) to produce cell extracts containing the kinases. Undesirable enzymatic activities present in the cell extracts (inter alia, phosphatases, nucleases, proteases, deaminases, oxidoreductases and/or hydrolases) are then removed (i.e., eliminated or inactivated) from the kinase-containing cell extracts, for example, by heating, without inactivating the kinase activities; in certain embodiments where heat is used to inactivate such undesirable enzymatic activities the kinases can be thermostable variants thereof resulting in a preparation containing kinase activity. In certain embodiments, undesirable enzymatic activity is eliminated (e.g. by physical separation, such as filtration, precipitation, capture, and/or chromatography) or inactivated (e.g. by temperature, pH, salt, detergent, alcohol or other solvents, and/or chemical inhibitors). NMPs or NDPs are then incubated with the preparations in the presence of an energy source (e.g. polyphosphate, such as hexametaphosphate) to produce NTPs in the cell-free reaction mixture. Minimally, a PPK and an energy source is required to convert NMPs or NDPs to NTPs. Optionally, NDK and nucleoside monophosphate kinases that are specific for each of the individual NMPs are included.

Polymerization into RNA

NTPs produced as described above can be subsequently or concurrently polymerized to RNA (either in the same reaction mixture or in a separate reaction mixture) using an RNA polymerase (e.g., bacteriophage T7 RNA polymerase) and an engineered template (e.g., DNA template, either expressed by the engineered cells and included as a cellular component of the cell-free reaction mixture, or later added to the cell-free reaction mixture). In some embodiments for producing eukaryotic mRNA, the DNA template is provided wherein the 3′ terminus of the sequence encoded in the template is followed by a polyadenylate sequence, so that the resulting RNA contains a polyadenylate (polyA) tail characteristic of eukaryotic mRNA. As used herein, the term untailed is used to describe “RNA lacking a polyA tail.” Alternatively, in DNA templates that do not encode a polyadenylate sequence positioned at the 3′ terminus of the sequence encoded in the template, the polyA tail can be added enzymatically by adding polyA polymerase, and incubating in the presence of ATP. ATP could be added directly, or produced by phosphorylating AMP and/or ADP using polyphosphate kinase and polyphosphate. Eukaryotic mRNA production further entails addition of a 5′ cap which can be accomplished using capping enzymes or capping reagents known in the art, as set forth with more specificity below. As used herein, the term uncapped means RNA lacking a cap.

### RNA to be Synthesized

As described below, in one embodiment, the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; and (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, vii) at least one DNA template encoding an mRNA with a polyA tail, viii) one or more capping reagents are added under conditions that produce mRNA, and further wherein, optionally ix) at least one deoxyribonuclease is added under conditions that digest the DNA following RNA production.

In a second embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, vii) at least one DNA template encoding an untailed RNA, and viii) one or more capping reagents are added under conditions that produce capped RNA; and further wherein optionally ix) at least one deoxyribonuclease is added under conditions that digest the DNA following RNA production; and d) (i) further incubating said reaction mixture produced in step (c) in the presence of a polyA polymerase and ATP, under conditions that produce mRNA or (ii) removing the RNA polymerase from the reaction mixture of step (c) by inactivation or physical separation followed by producing mRNA by further incubating the reaction mixture in the presence of polyA polymerase and ATP.

In a third embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase vii) at least one DNA template encoding an mRNA with a poly A tail, are added under conditions that produce uncapped RNA, and further wherein optionally viii) at least one deoxyribonuclease is added under conditions that digest the DNA following RNA production; and (d) exchanging buffer conditions from said reaction mixture from step (c) and incubating said reaction mixture in the presence of capping enzymes, GTP, and a methyl donor to produce mRNA.

In a fourth embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; (b) treating said reaction mixture under conditions wherein the RNA depolymerizing enzymes are eliminated or inactivated; (c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, and v) at least one nucleoside-diphosphate (NDP) kinase, under conditions wherein nucleotide triphosphates are produced and further wherein vi) at least one RNA polymerase, vii) at least one DNA template encoding an mRNA, are added under conditions that produce uncapped, untailed RNA, and further wherein, optionally viii) at least one deoxyribonuclease is added under conditions that digest the DNA following RNA production; d) (i) further incubating said reaction mixture produced in step (c) in the presence of a polyA polymerase and ATP, under conditions that produce uncapped RNA; or (ii) removing the RNA polymerase from the reaction mixture of step (c) by inactivation or physical separation followed by producing uncapped RNA by further incubating the reaction mixture in the presence of polyA polymerase and ATP; and e) exchanging buffer conditions from said reaction mixture from step (c) and incubating said reaction mixture in the presence of capping enzymes, GTP, and a methyl donor to produce mRNA.

In a fifth embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; and (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase, vii) at least one DNA template encoding an mRNA with a polyA tail, viii) one or more capping reagents are added under conditions that produce mRNA, and further wherein optionally ix) at least one deoxyribonuclease is added under conditions that digest the DNA following RNA production.

In a sixth embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor, and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase, vii) at least one DNA template encoding an untailed RNA, and viii) one or more capping reagents are added under conditions that produce capped RNA, and further wherein optionally ix) at least one deoxyribonuclease is added under conditions that digest the DNA following RNA production; and d) (i) further incubating said reaction mixture produced in step (c) in the presence of a polyA polymerase and ATP, under conditions that produce mRNA or (ii) removing the RNA polymerase from the reaction mixture of step (c) by inactivation or physical separation followed by producing mRNA by further incubating the reaction mixture in the presence of polyA polymerase and ATP.

In a seventh embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced, and further wherein vi) at least one RNA polymerase and vii) at least one DNA template encoding an mRNA with a poly A tail, are added under conditions that produce uncapped RNA; and further wherein optionally viii) at least one deoxyribonuclease is added under conditions that digest the DNA following RNA production; and (d) exchanging buffer conditions from said reaction mixture from step (c) and incubating said reaction mixture in the presence of capping enzymes, GTP, and a methyl donor to produce mRNA.

In an eighth embodiment the present disclosure features a cell-free reaction method for synthesizing a eukaryotic messenger ribonucleic acid (mRNA), the method comprising: (a) incubating in a reaction mixture cellular RNA and one or more enzymes that depolymerize RNA under conditions wherein the cellular RNA is substantially depolymerized, wherein the resulting first reaction mixture comprises nucleoside diphosphates; (b) treating said reaction mixture under conditions wherein the RNA depolymerizing enzymes are eliminated or inactivated; (c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, and v) at least one guanosine monophosphate (GMP) kinase, under conditions wherein nucleotide triphosphates are produced and further wherein vi) at least one RNA polymerase and vii) at least one DNA template encoding an mRNA, are added under conditions that produce uncapped, untailed RNA, and further wherein optionally viii) at least one deoxyribonuclease is added under conditions that digest the DNA following RNA production; (d) further incubating said reaction mixture produced in step (c) in the presence of a polyA polymerase and ATP, under conditions that produce uncapped RNA; or (ii) removing the RNA polymerase from the reaction mixture of step (c) by inactivation or physical separation followed by producing uncapped RNA by further incubating the reaction mixture in the presence of polyA polymerase and ATP; and e) exchanging buffer conditions from said reaction mixture from step (c) and incubating said reaction mixture in the presence of capping enzymes, GTP, and a methyl donor to produce mRNA.

In further embodiments, for each embodiment described above, steps (c)(i)-(c)(v) can be performed to produce nucleotide triphosphates before the remaining steps of (c), instead of concurrently.

In the embodiments described above, a second reaction mixture can be achieved by mixing a second reaction mixture or adding the recited components to the first reaction mixture to create the second reaction mixture.

Further, as described above, nucleotides produced by methods other than the depolymerization of cellular RNA as described in steps (a) and (b) of each embodiment (e.g., nucleotides derived from fermentation processes, chemical processes, or chemoenzymatic processes), can also be used as the nucleotides in step (c), thereby eliminating the need for steps (a) and (b) of each embodiment. Such nucleotides can include NMPs, NDPs, NTPs, or a mixture thereof. Further, such nucleotides may consist of one or more of unmodified nucleotides, modified nucleotides, or mixtures thereof. Such nucleotides may further consist of one or more unmodified NMPs and one or more modified NTPs, or an unmodified AMP, CMP, and GMP with pseudoUTP, or an unmodified AMP, GMP, pseudoUTP and 5-methyl CTP. The modified nucleotides can be added to cellular-derived nucleotides to achieve partially modified resulting mRNAs. Consistent with this, the use of such nucleotides can produce an mRNA as described in the embodiments herein.

In one embodiment, the methods are directed at cell-free RNA synthesis using cellular RNA in which the poly-A tail is encoded in the DNA template. The method comprises (a) lysing one or more cultures of cells that comprise kinases (e.g. nucleoside monophosphate (NMP) kinases, nucleoside diphosphate (NDP) kinases, polyphosphate kinases), a RNA polymerase, one or more capping enzymes, thereby producing one or more cell lysates, (b) combining in one or more reactions cellular RNA with an enzyme that depolymerizes RNA (e.g. Nuclease P1), and incubating that reaction under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) with one or more treatments that eliminate or inactivate undesired enzymatic activities, to produce 5′ NMP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate) and a DNA template containing a promoter operably linked to a nucleotide sequence encoding a mRNA and a polyadenylate sequence positioned at the 3′ terminus of the sequence encoded in the template, under conditions that result in production of nucleoside triphosphates and polymerization of the nucleoside triphosphates, thereby producing a cell-free reaction mixture that comprises uncapped RNA, and (e) exchanging the buffer and adding one or more capping enzyme preparations produced in step (c) to the reaction mixture of step (d), along with a methyl donor (e.g., S-adenosylmethionine), and incubating in the presence of GTP, thereby producing mRNA. In one aspect, the cell-free reaction mixture in step (d) comprises NMPs, kinases, an energy and phosphate source, a DNA template, and RNA polymerase. In a further aspect, the cell-free reaction mixture comprises NMPs, kinases, and an energy and phosphate source. The reaction mixture is then mixed with a DNA template and RNA polymerase. Optionally, after RNA synthesis the reaction mixture is treated with a deoxyribonuclease.

In a further embodiment, the methods are directed at cell-free RNA synthesis using cellular RNA in which a polyA tail is added enzymatically. The method comprises (a) lysing one or more cultures of cells that comprise kinases (e.g. nucleoside monophosphate (NMP) kinases, nucleoside diphosphate (NDP) kinases, polyphosphate kinases), a RNA polymerase, a PolyA polymerase, one or more capping enzymes, thereby producing one or more cell lysates, (b) combining in one or more reactions cellular RNA with an enzyme that depolymerizes RNA (e.g. Nuclease P1), and incubating that reaction under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) with one or more treatments that eliminate or inactivate undesired enzymatic activities, to produce 5′ NMP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate) and a DNA template containing a promoter operably linked to a nucleotide sequence encoding a mRNA, under conditions that result in production of nucleoside triphosphates and polymerization of the nucleoside triphosphates, thereby producing a cell-free reaction mixture that comprises uncapped, untailed RNA, (e) treating the cell-free reaction mixture with a deoxyribonuclease; (f) adding a polyA tail enzymatically in the presence of polyA polymerase and ATP to produce uncapped RNA, and (g) exchanging the buffer and adding one or more capping enzyme preparations produced in step (c) to the reaction mixture of step (f), along with a methyl donor (e.g., S-adenosylmethionine), and incubating in the presence of GTP, thereby producing mRNA. In one aspect, the cell-free reaction mixture comprises NMPs, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the DNA template. The method comprises (a) lysing one or more cultures of cells that comprise cellular RNA, kinases (e.g. nucleoside monophosphate (NMP) kinases, nucleoside diphosphate (NDP) kinases, polyphosphate kinases), a RNA polymerase, thereby producing one or more cell lysates, (b) combining the one or more cell lysates produced in step (a) comprising cellular RNA with an enzyme that depolymerizes RNA (e.g. Nuclease P1), and incubating the cell lysate under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) comprising kinases, and RNA polymerase with one or more treatments that eliminate or inactivate undesired enzymatic activities, to produce 5′ NMP preparations and enzyme preparations (d) combining the one or more preparations produced in step (c) in the cell-free reaction mixture comprising kinases and RNA polymerase and incubating the cell-free reaction mixture in the presence of an energy and phosphate source (e.g. polyphosphate), capping reagent, a DNA template containing a promoter operably linked to a nucleotide sequence encoding a mRNA and a polyadenylate sequence positioned at the 3? terminus of the sequence encoded in the template, and capping reagent under conditions that result in production of nucleoside triphosphates and polymerization of the nucleoside triphosphates, thereby producing a cell-free reaction mixture that comprises mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase. Optionally, after RNA synthesis the reaction mixture is treated with a deoxyribonuclease.

In a further embodiment, the methods are directed at cell-free RNA synthesis using cell lysates in which the poly-A tail is added enzymatically. The method comprises (a) lysing one or more cultures of cells that comprise cellular RNA, kinases (e.g. nucleoside monophosphate (NMP) kinases, nucleoside diphosphate (NDP) kinases, polyphosphate kinases), a RNA polymerase, a polyA polymerase, thereby producing one or more cell lysates, (b) combining the one or more cell lysates produced in step (a) comprising cellular RNA with an enzyme that depolymerizes RNA (e.g. Nuclease P1), and incubating that cell lysate under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) comprising kinases, RNA polymerase, and polyA polymerase with one or more treatments that eliminate or inactivate undesired enzymatic activities, to produce 5′NMP preparations and enzyme preparations (d) combining the one or more preparations produced in step (c) in the cell-free reaction mixture comprising kinases and RNA polymerase and incubating the cell-free reaction mixture in the presence of an energy and phosphate source (e.g. polyphosphate), a DNA template containing a promoter operably linked to a nucleotide sequence encoding an untailed RNA, and capping reagent, under conditions that result in production of nucleoside triphosphates and polymerization of the nucleoside triphosphates, thereby producing a cell-free reaction mixture that comprises untailed RNA, (e) treating the cell-free reaction mixture with a deoxyribonuclease; and (f) adding a polyA tail enzymatically in the presence of polyA polymerase and ATP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template containing a promoter in which the poly-A tail is encoded in the template. The method comprises (a) lysing one or more cultures of cells that comprise kinases (e.g. nucleoside monophosphate (NMP) kinases, nucleoside diphosphate (NDP) kinases, polyphosphate kinases), a RNA polymerase, a DNA template containing a promoter operably linked to a nucleotide sequence encoding a mRNA and a polyadenylate sequence positioned at the 3′ terminus of the sequence encoded in the template, one or more capping enzymes, thereby producing one or more cell lysates, (b) combining in one or more reactions cellular RNA with an enzyme that depolymerizes RNA (e.g. Nuclease P1), and incubating that reaction under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) with one or more treatments that eliminate or inactivate undesired enzymatic activities, to produce 5′-NMP preparations, enzyme preparations, and DNA template preparations (d) incubating the DNA template with a restriction endonuclease, e.g. a Type IIS restriction endonuclease, that cleaves immediately 3′ to the encoded polyA tail, to produce a preparation of linearized DNA template, and subsequently inactivating or removing the restriction endonuclease (e) combining the one or more preparations produced in step (c) in the cell-free reaction mixture comprising kinases and RNA polymerase and incubating the cell-free reaction mixture in the presence of an energy and phosphate source (e.g. polyphosphate) and a DNA template under conditions that result in production of nucleoside triphosphates and polymerization of the nucleoside triphosphates, thereby producing a cell-free reaction mixture that comprises uncapped RNA, and (e) exchanging the buffer and adding one or more capping enzyme preparations produced in step (c) to the reaction mixture of step (d), along with a methyl donor (e.g., S-adenosylmethionine) and incubating in the presence of GTP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template containing a promoter in which the poly-A tail is added enzymatically. The method comprises RNA synthesis methods can comprise (a) lysing one or more cultures of cells that comprise kinases (e.g. nucleoside monophosphate (NMP) kinases, nucleoside diphosphate (NDP) kinases, polyphosphate kinases), a RNA polymerase, a PolyA polymerase, a DNA template containing a promoter operably linked to a nucleotide sequence encoding an untailed RNA, one or more capping enzymes, thereby producing one or more cell lysates, (b) combining in one or more reactions cellular RNA with an enzyme that depolymerizes RNA (e.g. Nuclease P1), and incubating that reaction under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) with one or more treatments that eliminate or inactivate undesired enzymatic activities, to produce 5′-NMP preparations, enzyme preparations, and DNA template preparations, Incubating the DNA template with a restriction endonuclease, e.g. a Type IIS restriction endonuclease, that cleaves immediately 3′ to the encoded untailed RNA, to produce a preparation of linearized DNA template, and subsequently inactivating or removing the restriction endonuclease (d) combining the one or more preparations produced in step (c) in the cell-free reaction mixture comprising kinases and RNA polymerase and incubating the cell-free reaction mixture in the presence of an energy and phosphate source (e.g. polyphosphate) and DNA template under conditions that result in production of nucleoside triphosphates and polymerization of the nucleoside triphosphates, thereby producing a cell-free reaction mixture that comprises uncapped, untailed RNA, (e) treating the cell-free reaction mixture with a deoxyribonuclease, (f) adding a polyA tail enzymatically in the presence of polyA polymerase and ATP, thereby producing a cell-free reaction mixture that comprises uncapped RNA, (g) exchanging the buffer and adding one or more capping enzyme preparations produced in step (c) to the reaction mixture of step (f), along with a methyl donor (e.g., S-adenosylmethionine) and incubating in the presence of GTP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the template. The method comprises (a) lysing one or more cultures of cells that comprise cellular RNA, kinases (e.g. nucleoside monophosphate (NMP) kinases, nucleoside diphosphate (NDP) kinases, polyphosphate kinases), a RNA polymerase, a DNA template containing a promoter operably linked to a nucleotide sequence encoding a mRNA and a polyadenylate sequence positioned at the 3′ terminus of the sequence encoded in the template, thereby producing one or more cell lysates, (b) combining the one or more cell lysates produced in step (a) comprising cellular RNA with an enzyme that depolymerizes RNA (e.g. Nuclease P1), and incubating that cell lysate under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) comprising kinases RNA polymerase, and one or more capping enzymes with one or more treatments that eliminate or inactivate undesired enzymatic activities, to produce 5′-NMP preparations, enzyme preparations, and DNA template preparations, (d) Incubating the DNA template with a restriction endonuclease, e.g. a Type IIS restriction endonuclease, that cleaves immediately 3′ to the encoded polyA tail, to produce a preparation of linearized DNA template, and subsequently inactivating or removing the restriction endonuclease; (e) combining the one or more preparations produced in (c) in the cell-free reaction mixture comprising kinases and RNA polymerase and incubating the cell-free reaction mixture in the presence of an energy and phosphate source (e.g. polyphosphate), capping reagent, and DNA template under conditions that result in production of nucleoside triphosphates and polymerization of the nucleoside triphosphates, thereby producing a cell-free reaction mixture that comprises mRNA. Optionally, after RNA synthesis the reaction mixture is treated with a deoxyribonuclease. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template in which the poly-A tail is added enzymatically. The method comprises RNA synthesis methods can comprise (a) lysing one or more reactions one or more cultures of cells that comprise cellular RNA, kinases (e.g. nucleoside monophosphate (NMP) kinases, nucleoside diphosphate (NDP) kinases, polyphosphate kinases), a RNA polymerase, a PolyA polymerase, a DNA template containing a promoter operably linked to a nucleotide sequence encoding an untailed RNA, thereby producing one or more cell lysates, (b) combining the one or more cell lysates produced in step (a) comprising cellular RNA with an enzyme that depolymerizes RNA (e.g. Nuclease P1), and incubating that cell lysate under conditions that result in depolymerization of RNA, thereby producing a cell-free reaction mixture that comprises 5′ nucleoside monophosphates, (c) treating (i) the cell-free reaction mixture comprising 5′ nucleoside monophosphates produced in step (b) and (ii) the one or more cell lysates produced in step (a) comprising kinases and RNA polymerase with one or more treatments that eliminate or inactivate undesired enzymatic activities, to produce 5′-NMP preparations, enzyme preparations, and DNA template preparations, (d) Incubating the DNA template with a restriction endonuclease, e.g. a Type IIS restriction endonuclease, that cleaves immediately 3′ to the encoded untailed RNA, to produce a preparation of linearized DNA template, and subsequently inactivating or removing the restriction endonuclease; (e) combining the one or more preparations produced in (c) in the cell-free reaction mixture comprising kinases and RNA polymerase and incubating the cell-free reaction mixture in the presence of an energy and phosphate source (e.g. polyphosphate), capping reagent, and DNA template, under conditions that result in production of nucleoside triphosphates and polymerization of the nucleoside triphosphates, thereby producing a cell-free reaction mixture that comprises untailed RNA, (f) treating the cell-free reaction mixture with a deoxyribonuclease, and (g) adding a polyA tail enzymatically by incubating in the presence of polyA polymerase and ATP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the DNA template. The method comprises (a) lysing one or more cultures of cells that comprise kinases (e.g. nucleoside monophosphate (NMP) kinases, nucleoside diphosphate (NDP) kinases, polyphosphate kinases), a RNA polymerase, one or more capping enzymes, thereby producing one or more cell lysates, (b) combining in one or more reactions cellular RNA with an enzyme that depolymerizes RNA (e.g. PNPase), and incubating that reaction under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) with one or more treatments that eliminate or inactivate undesired enzymatic activities, to produce NDP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate) and a DNA template containing a promoter operably linked to a nucleotide sequence encoding a mRNA and a polyadenylate sequence positioned at the 3′ terminus of the sequence encoded in the template, under conditions that result in production of nucleoside triphosphates and polymerization of the nucleoside triphosphates, thereby producing a cell-free reaction mixture that comprises uncapped RNA, (e) exchanging the buffer and adding one or more capping enzyme preparations produced in step (c) to the reaction mixture of step (d), along with a methyl donor (e.g., S-adenosylmethionine) and incubating in the presence of GTP, thereby producing mRNA. Optionally, after RNA synthesis the reaction mixture is treated with a deoxyribonuclease. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template containing a promoter in which the poly-A tail is added enzymatically. The method comprises (a) lysing one or more cultures of cells that comprise kinases (e.g. nucleoside monophosphate (NMP) kinases, nucleoside diphosphate (NDP) kinases, polyphosphate kinases), a RNA polymerase, a PolyA polymerase, one or more capping enzymes, thereby producing one or more cell lysates, (b) combining in one or more reactions cellular RNA with an enzyme that depolymerizes RNA (e.g. PNPase), and incubating that reaction under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) with one or more treatments that eliminate or inactivate undesired enzymatic activities, to produce 5′NDP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate) and a DNA template containing a promoter operably linked to a nucleotide sequence encoding an untailed RNA, under conditions that result in production of nucleoside triphosphates and polymerization of the nucleoside triphosphates, thereby producing a cell-free reaction mixture that comprises uncapped, untailed RNA, (e) treating the cell-free reaction mixture with a deoxyribonuclease; (f) adding a polyA tail enzymatically by adding polyA polymerase and incubating in the presence of ATP, thereby producing uncapped RNA; (g) exchanging the buffer and adding one or more capping enzyme preparations produced in step (c) to the reaction mixture of step (f), along with a methyl donor (e.g., S-adenosylmethionine) and incubating in the presence of GTP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the DNA template. The methods comprise (a) lysing one or more cultures of cells that comprise cellular RNA, kinases (e.g. nucleoside monophosphate (NMP) kinases, nucleoside diphosphate (NDP) kinases, polyphosphate kinases), a RNA polymerase, thereby producing one or more cell lysates, (b) combining the one or more cell lysates produced in step (a) comprising cellular RNA with an enzyme that depolymerizes RNA (e.g. PNPase), and incubating that cell lysate under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) comprising kinases and RNA polymerase, with one or more treatments that eliminate or inactivate undesired enzymatic activities, to produce 5′-NDP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate), capping reagent, and a DNA template containing a promoter operably linked to a nucleotide sequence encoding a mRNA and a polyadenylate sequence positioned at the 3′ terminus of the sequence encoded in the template, under conditions that result in production of nucleoside triphosphates and polymerization of the nucleoside triphosphates, thereby producing a cell-free reaction mixture that comprises mRNA. Optionally, after RNA synthesis the reaction mixture is treated with a deoxyribonuclease. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template containing a promoter in which the poly-A tail is added enzymatically. The method comprises (a) lysing one or more cultures of cells that comprise cellular RNA, kinases (e.g. nucleoside monophosphate (NMP) kinases, nucleoside diphosphate (NDP) kinases, polyphosphate kinases), a RNA polymerase, a PolyA polymerase, thereby producing one or more cell lysates, (b) combining the one or more cell lysates produced in step (a) comprising cellular RNA with an enzyme that depolymerizes RNA (e.g. PNPase), and incubating that cell lysate under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) comprising kinases RNA polymerase, and one or more capping enzymes with one or more treatments that eliminate or inactivate undesired enzymatic activities, to produce 5′-NMP preparations and enzyme preparations, (d) combining the one or more preparations produced in step (c) in the cell-free reaction mixture comprising kinases and RNA polymerase and incubating the cell-free reaction mixture in the presence of an energy and phosphate source (e.g. polyphosphate), capping reagent, and a DNA template containing a promoter operably linked to a nucleotide sequence encoding an untailed RNA, under conditions that result in production of nucleoside triphosphates and polymerization of the nucleoside triphosphates, thereby producing a cell-free reaction mixture that comprises untailed RNA, (e) treating the cell-free reaction mixture with a deoxyribonuclease; (f) adding a polyA tail enzymatically by adding polyA polymerase and incubating in the presence of ATP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the DNA template. The methods comprise (a) lysing one or more cultures of cells that comprise kinases (e.g. nucleoside monophosphate (NMP) kinases, nucleoside diphosphate (NDP) kinases, polyphosphate kinases), a RNA polymerase, a DNA template containing a promoter operably linked to a nucleotide sequence encoding a mRNA and a polyadenylate sequence positioned at the 3′ terminus of the sequence encoded in the template, one or more capping enzymes, thereby producing one or more cell lysates, (b) combining in one or more reactions cellular RNA with an enzyme that depolymerizes RNA (e.g. PNPase), and incubating that reaction under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) with one or more treatments that eliminate or inactivate undesired enzymatic activities, to produce 5′NDP preparations, enzyme preparations, and DNA template preparations, d) incubating the DNA template with a restriction endonuclease, e.g. a Type IIS restriction endonuclease, that cleaves immediately 3′ to the encoded polyA tail, to produce a preparation of linearized DNA template, and subsequently inactivating or removing the restriction endonuclease (e) combining the one or more preparations produced in step (c) in the cell-free reaction mixture comprising kinases and RNA polymerase and incubating the cell-free reaction mixture in the presence of an energy and phosphate source (e.g. polyphosphate) and DNA template under conditions that result in production of nucleoside triphosphates and polymerization of the nucleoside triphosphates, thereby producing a cell-free reaction mixture that comprises uncapped RNA, and (f) exchanging the buffer and adding one or more capping enzyme preparations produced in step (c) to the reaction mixture of step (e), along with a methyl donor (e.g., S-adenosylmethionine), and incubating in the presence of and GTP, thereby producing mRNA. Optionally, after RNA synthesis the reaction mixture is treated with a deoxyribonuclease. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates in which the poly-A tail is encoded in the DNA template. The methods comprise (a) lysing one or more cultures of cells that comprise kinases (e.g. nucleoside monophosphate (NMP) kinases, nucleoside diphosphate (NDP) kinases, polyphosphate kinases), a RNA polymerase, a PolyA polymerase, a DNA template containing a promoter operably linked to a nucleotide sequence encoding an untailed RNA, and one or more capping enzymes, thereby producing one or more cell lysates, (b) combining in one or more reactions cellular RNA with an enzyme that depolymerizes RNA (e.g. PNPase), and incubating that reaction under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) with one or more treatments that eliminate or inactivate undesired enzymatic activities, to produce 5′NDP preparations, enzyme preparations, and DNA template preparations, (d) incubating the DNA template with a restriction endonuclease, e.g. a Type IIS restriction endonuclease, that cleaves immediately 3′ to the encoded untailed RNA, to produce a preparation of linearized DNA template, and subsequently inactivating or removing the restriction endonuclease (e) combining the one or more preparations produced in (c) in the cell-free reaction mixture comprising kinases and RNA polymerase and incubating the cell-free reaction mixture in the presence of an energy and phosphate source (e.g. polyphosphate) and DNA template under conditions that result in production of nucleoside triphosphates and polymerization of the nucleoside triphosphates, thereby producing a cell-free reaction mixture that comprises uncapped, untailed RNA, (f) treating the cell-free reaction mixture with a deoxyribonuclease; (g) adding a polyA tail enzymatically by adding polyA polymerase and incubating in the presence of ATP, thereby producing uncapped RNA; (h) exchanging the buffer and adding one or more capping enzyme preparations produced in step (c) to the reaction mixture of step (e), along with a methyl donor (e.g., S-adenosylmethionine) and incubating in the presence of GTP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, RNA synthesis methods can comprise (a) lysing one or more reactions one or more cultures of cells that comprise cellular RNA, kinases (e.g. nucleoside monophosphate (NMP) kinases, nucleoside diphosphate (NDP) kinases, polyphosphate kinases), a RNA polymerase, a DNA template containing a promoter operably linked to a nucleotide sequence encoding a mRNA and a polyadenylate sequence positioned at the 3′ terminus of the sequence encoded in the template, thereby producing one or more cell lysates, (b) combining the one or more cell lysates produced in step (a) comprising cellular RNA with an enzyme that depolymerizes RNA (e.g. PNPase), and incubating that cell lysate under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) comprising kinases RNA polymerase, and one or more capping enzymes with one or more treatments that eliminate or inactivate undesired enzymatic activities, to produce 5′ NDP preparations, enzyme preparations, and DNA template preparations, d) incubating the DNA template with a restriction endonuclease, e.g. a Type IIS restriction endonuclease, that cleaves immediately 3′ to the encoded polyA tail, to produce a preparation of linearized DNA template, and subsequently inactivating or removing the restriction endonuclease; (e) combining the one or more preparations produced in step (c) in the cell-free reaction mixture comprising kinases and RNA polymerase and incubating the cell-free reaction mixture in the presence of an energy and phosphate source (e.g. polyphosphate), capping reagent, and DNA template under conditions that result in production of nucleoside triphosphates and polymerization of the nucleoside triphosphates, thereby producing a cell-free reaction mixture that comprises mRNA. Optionally, after RNA synthesis the reaction mixture is treated with a deoxyribonuclease. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

In a further embodiment, the methods are directed at cell-free RNA synthesis using cell lysates that include a DNA template in which the poly-A tail is added enzymatically. The methods comprise (a) lysing one or more reactions one or more cultures of cells that comprise cellular RNA, kinases (e.g. nucleoside monophosphate (NMP) kinases, nucleoside diphosphate (NDP) kinases, polyphosphate kinases), a RNA polymerase, a PolyA polymerase, a DNA template containing a promoter operably linked to a nucleotide sequence encoding an untailed RNA, thereby producing one or more cell lysates, (b) combining the one or more cell lysates produced in step (a) comprising cellular RNA with an enzyme that depolymerizes RNA (e.g. PNPase), and incubating that cell lysate under conditions that result in depolymerization of RNA, thereby producing a cell-free reaction mixture that comprises 5′ nucleoside diphosphates, (c) treating (i) the cell-free reaction mixture comprising 5′ nucleoside diphosphates produced in step (b) and (ii) the one or more cell lysates produced in step (a) comprising kinases and RNA polymerase with one or more treatments that eliminate or inactivate undesired enzymatic activities, to produce 5′NDP preparations, enzyme preparations, and DNA template preparations (d) incubating the DNA template with a restriction endonuclease, e.g. a Type IIS restriction endonuclease, that cleaves immediately 3′ to the encoded untailed RNA, to produce a preparation of linearized DNA template, and subsequently inactivating or removing the restriction endonuclease (e) combining the one or more preparations produced in steps (c-d) in the cell-free reaction mixture comprising kinases and RNA polymerase and incubating the cell-free reaction mixture in the presence of an energy and phosphate source (e.g. polyphosphate) and capping reagent, under conditions that result in production of nucleoside triphosphates and polymerization of the nucleoside triphosphates, thereby producing a cell-free reaction mixture that comprises untailed RNA, (g) treating the cell-free reaction mixture with a deoxyribonuclease; (h) adding a polyA tail enzymatically by adding polyA polymerase and incubating in the presence of ATP, thereby producing mRNA. In one aspect, the cell-free reaction mixture comprises NMP, kinases, a source of phosphates, a DNA template, and RNA polymerase. In a further aspect, the cell-free reaction mixture comprises NMPs, kinases, and a phosphate source. The reaction mixture is then mixed with a DNA template and RNA polymerase.

Further embodiments of synthesizing mRNA include, including an internal ribosome entry site (IRES) in any of the uncapped mRNA produced from cellular RNA depolymerized to NMPs or NDPs, respectively, as discussed above. In still other embodiments, the uncapped mRNA produced by any of the previous embodiments is subsequently capped using capping enzyme(s) to produce a capped mRNA. In still other embodiments, the uncapped mRNA produced from cellular RNA depolymerized to NMPs or NDPs, respectively, is subsequently capped using capping enzyme(s) to produce a capped mRNA. In still other embodiments, the RNA-polymerase-containing step of any of the previous embodiments also includes a cap analog, thereby producing capped mRNA instead of uncapped RNA and obviating the need for subsequent enzymatic capping step(s).

As discussed above, examples of RNA end products include preferably messenger RNA (mRNA). In some embodiments, the concentration of RNA end product (biosynthesized RNA) is at least 1 g/L to 50 g/L. For example, the concentration of RNA end product can be 1, 5, 10, 15, 20, 25, 30, 35, 40, 45, or 50 g/L, or more. In many embodiments, the concentration of RNA end product is at least 1 g/L. Single batches can be up to or exceeding 10,000 L.

### Cell-Free Production

“Cell-free production” is the use of biological processes for the synthesis of a biomolecule or chemical compound without using living cells. Initially in such methods, cells are lysed, resulting in cell lysates. Unpurified (crude) portions or partially purified portions, both containing enzymes, can be used for producing a desired product. In some embodiments, enzymes used in such processes are purified enzymes that can be added to cell lysates. As a non-limiting example, cells can be cultured, harvested, and lysed by high-pressure homogenization or other cell lysis methods (e.g., chemical cell lysis). Cell-free reactions can be conducted in a batch or fed-batch mode. In some instances, enzymatic pathways fill a reactor's working volume and can be more dilute than in the intracellular environment. Yet substantially all of the cellular catalysts can be provided thereby, including catalysts that are membrane-associated.

It should be understood that while many of the embodiments described herein refer to “lysing cultured cells” that comprise particular enzymes, the phrase is intended to encompass lysing a clonal population of cells obtained from a single culture (e.g., containing all the enzymes needed to synthesize RNA) as well as lysing more than one clonal population of cells, each obtained from different cell cultures (e.g., each containing one or more enzymes needed to synthesize RNA and/or the cellular RNA substrate). For example, in some embodiments, a population of cells (e.g., engineered cells) expressing a particular kinase can be cultured together and used to produce one cell lysate, and another population of cells (e.g., engineered cells) expressing a different kinase can be cultured together and used to produce another cell lysate. These two or more cell lysates, each comprising different kinase, can then be combined for use in a cell-free mRNA biosynthesis method of the present disclosure. In embodiments of this invention wherein heat is used to inactivate undesired enzymatic activities in the presence of enzymes, such as kinases whose enzymatic activities are desired to be maintained, thermostable variants of such enzymes, such as kinases or, are advantageously employed.

### Depolymerization of Biomass Ribonucleic Acid to Nucleoside Monophosphates

The present disclosure is based on the conversion of RNA from biomass (e.g., endogenous cellular RNA) to desired synthetic mRNA through a cell-free process involving a series of enzymatic reactions. First, RNA (e.g., endogenous RNA) present in a reaction mixture, derived from cellular RNA, is converted to its constituent monomers by a nuclease. As used herein and understood in the art, the term “biomass” is intended to mean the total mass of cellular materials and includes, but is not limited to, carbohydrate, DNA, lipid, protein, RNA, and fragments thereof. Optionally, the RNA can be crudely purified before conversion to monomers. RNA from biomass (e.g., endogenous RNA) typically includes ribosomal RNA (rRNA), messenger RNA (mRNA), transfer RNA (tRNA), other RNAs, or a combination thereof. Depolymerization or degradation of RNA with an appropriate nuclease, for example, a nuclease that produces 5′-nucleoside monophosphates (5′-NMPs) results in a pool of 5′-NMPs, also referred to simply as “monomers.” These monomers, which are converted to nucleoside diphosphates, which are further converted to nucleoside triphosphates, are used as starting material for downstream polymerization/synthesis of an mRNA. In some embodiments, the monomers have a modified backbone lineage or are alternative bases, i.e. thioate, and are depolymerized with a specialized nuclease and phosphorylated with a specialized kinase. Production of commercial quantities of NTPs is contemplated as described in PCT/US2018/05535 entitled “Methods and Compositions for Nucleoside Triphosphate and Ribonucleic Acid Production”, incorporated by reference herein in its entirety.

The amount of RNA (e.g., endogenous RNA) required to synthesize an mRNA can vary, depending on, for example, the desired length and yield of a particular mRNA as well as the nucleotide composition of the mRNA relative to the nucleotide composition of the RNA of the cell (e.g., endogenous RNA from an E. coli cell or a yeast cell). Typically, for a bacterial cell, for example, RNA (e.g., endogenous RNA) content ranges from 5-50% of the total cell mass, whereas for eukaryotic cells the amount is about 20%. The mass percent of the starting material can be calculated, for example, using the following equation: (kilogram (kg) of RNA/kilogram of dry cell weight)×100%.

Endogenous RNA can be depolymerized or degraded into its constituent monomers by chemical or enzymatic means. Chemical hydrolysis of RNA, however, typically produces 2′- and 3′-NMPs, which cannot be polymerized into RNA and thus are less advantageous than enzymatic degradation methods. Thus, the methods, compositions, and systems as provided herein primarily use enzymes for depolymerizing endogenous RNA. An “enzyme that depolymerizes RNA” catalyzes hydrolysis of phosphodiester bonds between two nucleotides in an RNA molecule. Thus, “an enzyme that depolymerizes RNA” converts RNA (cellular RNA) into its monomeric form, either nucleoside monophosphates (NMPs) or nucleoside diphosphates (NDPs). Depending on the enzyme, enzymatic depolymerization of RNA can yield 3′-NMPs, 5′-NMPs, a combination of 3′-NMPs and 5′-NMPs, or 5′-NDPs. Because it is not possible to polymerize 3′-NTPs (converted from 3′-NDPs, which are converted from 3′-NMPs), enzymes that yield 5′-NMPs (which are then converted to 5′-NDPs, and then 5′-NTPs) or 5′-NDPs (which are then converted to 5′-NTPs) such as Nuclease P1 or PNPase are preferred. In some embodiments, enzymes that yield 3′-NMPs are removed from genomic DNA of the engineered cell to increase efficiency of RNA production. In some embodiments, the enzyme used for RNA depolymerization is Nuclease P1. In some embodiments, the concentration of Nuclease P1 used is 0.1-3.0 mg/mL (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mg/mL). In some embodiments, the concentration of Nuclease P1 used is 1-3 mg/mL.

Examples of enzymes that depolymerize RNA include, without limitation, nucleases (e.g., Nuclease P1), including ribonucleases (RNases, e.g., RNase R) and phosphodiesterases. Nucleases catalyze the degradation of nucleic acid into smaller components (e.g., monomers, also referred to as nucleoside monophosphates, or oligonucleotides). Phosphodiesterases catalyze degradation of phosphodiester bonds. These enzymes that depolymerize RNA can be encoded by full-length genes or by gene fusions (e.g., DNA that includes at least two different genes (or fragments of genes) encoding at two different enzymatic activities).

RNase functions in cells to regulate RNA maturation and turn over. Each RNase has specific substrate preferences. Thus, in some embodiments, a combination of different RNases, or a combination of different nucleases, generally, can be used to depolymerize biomass-derived RNA (e.g., endogenous RNA). For example, 1-2, 1-3, 1-4, 1-5, 1-6, 1-7, 1-8, 1-9, or 1-10 different nucleases can be used in combination to depolymerize RNA. In some embodiments, at least 1, at least 2, at least 3, at least 4, at least 5, at least 6, at least 7, at least 8, at least 9, or at least 10 different nucleases can be used in combination to depolymerize RNA. Non-limiting examples of nucleases for use as provided herein are included in Table 1. In some embodiments, the nuclease used is Nuclease P1.

TABLE 1
Examples of Enzymes for RNA Depolymerization

| Enzyme | Organism | EC # | UniProt | Reference |
|---|---|---|---|---|
| Nuclease P1 | Penicillium citrinum | 3.1.30.1 | P24289 | 1, 2, 3 |
| (P1 Nuclease) | | | | |
| RNase II | Escherichia coli | 3.1.13.1 | P30850 | 4, 5 |
| RNase III | Escherichia coli | 3.1.26.3 | P0A7Y0 | 6, 7, 8 |
| RNase R | Pseudomonas putida | 3.1.13.— | R9V9M9 | 9 |
| | or | | P21499 | |
| | Escherichia coli | | | |
| RNase JI | Bacillus subtilis | 3.1.4.1 | Q45493 | 10, 11 |
| NucA | Serratia marcescens | 3.1.30.2 | P13717 | 12, 13, 14 |
| RNase T | Escherichia coli | 3.1.27.3 | P30014 | 15, 16, 17 |
| RNase E | Escherichia coli | 3.1.26.12 | P21513 | 18, 19 |
| PNPase | Escherichia coli | 2.7.7.8 | P05055 | 55 |

Enzymes that depolymerize RNA (e.g., RNases) can be endogenous to a host cell (host-derived), or they can be encoded by engineered nucleic acids exogenously introduced into a host cell (e.g., on an episomal vector or integrated into the genome of the host cell). Alternatively, enzymes can be added to the reaction as isolated protein, including commercially sourced isolated protein. As other alternatives, partially purified enzymes can be used in the reaction, including enzymes that are partially purified from cells that endogenously produce the enzyme or cells that are engineered to produce the enzyme.

For incubating cellular RNA in a cell-free reaction mixture, conditions that result in depolymerization of RNA are known in the art or can be determined by one of ordinary skill in the art, taking into consideration, for example, optimal conditions for a particular nuclease (e.g., Nuclease P1) activity, including pH, temperature, length of time, and salt concentration of the cell lysate as well as any exogenous cofactors. Examples for these reaction conditions include those described previously (see, e.g., Wong et al., 1983, J. Am. Chem. Soc. 105: 115-117, European Patent No. EP1587947B1, or Cheng and Deutscher, 2002, J Biol Chem. 277:21624-21629).

In some embodiments, metal ions (e.g., Zn²⁺, Mg²⁺) are depleted from the depolymerization reaction. In some embodiments, the concentration of metal ion (e.g., Zn²⁺, Mg²⁺) is 8 mM or less (e.g., less than 8 mM, less than 7 mM, less than 6 mM, less than 5 mM, less than 4 mM, less than 3 mM, less than 2 mM, less than 1 mM, less than 0.5 mM, less than 0.1 mM and less than 0.05 mM). In some embodiments, the concentration of metal ion (e.g., Zn²⁺, Mg²⁺) is 0.1 mM-8 mM, 0.1 mM-7 mM, or 0.1 mM-5 mM. In some embodiments, the metal ion is Zn²⁺.

The pH of a cell lysate during an RNA depolymerization reaction can have a value of 3 to 8. In some embodiments, the pH value of a cell lysate is 3-8, 4-8, 5-8, 6-8, 7-8, 3-7, 4-7, 5-7, 6-7, 3-6, 4-6, 5-6, 3-5, 3-4, or 4-5. In some embodiments, the pH value of a cell lysate is 3.0, 3.5, 4.0, 4.5, 5.0, 5.5, 6.0, 6.5, 7.0, 7.5, or 8.0. In some embodiments, the pH value of a cell lysate is 5.0, 5.1, 5.2, 5.3, 5.4, or 5.5. In some advantageous embodiments, the pH is 5.8. The pH of a cell lysate can be adjusted, as needed.

The temperature of a cell lysate during a RNA depolymerization reaction can be 15° C. to 99° C. In some embodiments, the temperature of a cell lysate during an RNA depolymerization reaction is 15-95° C., 15-90° C., 15-80° C., 15-70° C., 15-60° C., 15-50° C., 15-40° C. 15-30° C., 25-95° C., 25-90° C., 25-80° C., 25-70° C., 25-60° C., 25-50° C., 25-40° C., 25-30° C., 30-95° C., 30-90° C., 30-80° C., 30-70° C., 30-60° C., 30-50° C., 40-95° C., 40-90° C., 40-80° C., 40-70° C., 40-60° C., 40-50° C., 50-95° C., 50-90° C., 50-80° C., 50-70° C., 50-60° C., 60-95° C., 60-90° C., 60-80° C., or 60-70° C. In some embodiments, the temperature of a cell lysate during an RNA depolymerization reaction is 70° C. In some embodiments, the temperature of a cell lysate during an RNA depolymerization reaction is 15° C., 25° C., 32° C., 37° C., 40° C., 42° C., 45° C., 50° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., or 99° C.

In some embodiments, a cell-free reaction mixture during an RNA depolymerization reaction is incubated for 24 hours at a temperature of 70° C. In some embodiments, a reaction mixture during a RNA depolymerization reaction is incubated for 5-30 min at a temperature of 70° C. In some embodiments, a reaction mixture during a RNA depolymerization reaction has a pH of 5-5.5 and is incubated for 15 minutes at a temperature of 70° C. In some embodiments, a reaction mixture during a RNA depolymerization reaction may be incubated under conditions that result in greater than 65% conversion of RNA to NDP or RNA to 5′-NMPs. In some embodiments, RNA is converted to NDP or 5′-NMPs at a rate of (or at least) 50 mM/hr, 100 mM/hr or 200 mM/hr. In other embodiments, a reaction mixture during an RNA depolymerization reaction is incubated at a higher temperature (for example, 50° C.-70° C.).

A cell lysate produced for effecting a RNA depolymerization reaction can be incubated for 5 minutes (min) to 72 hours (hrs). In some embodiments, a cell lysate during an RNA depolymerization reaction is incubated for 5-10 min, 5-15 min, 5-20 min, 5-30 min, or 5 min-48 hrs. For example, a cell lysate during an RNA depolymerization reaction can be incubated for 5 min, 10 min, 15 min, 20 min. 25 min, 30 min, 45 min, 1 hr, 2 hrs. 3 hrs, 4 hrs, 5 hrs, 6 hrs, 7 hrs, 8 hrs, 9 hrs, 10 hrs, 11 hrs, 12 hrs, 18 hrs, 24 hrs, 30 hrs, 36 hrs. 42 hours, or 48 hours. In some embodiments, a cell lysate during an RNA depolymerization reaction is incubated for 24 hours at a temperature of 37° C. In some embodiments, a cell lysate during an RNA depolymerization reaction is incubated for 5-10 min at a temperature of 70° C. In some embodiments, a cell lysate during an RNA depolymerization reaction has a pH of 5-5.5 and is incubated for 15 minutes at a temperature of 70° C. In some embodiments, a cell lysate during an RNA depolymerization reaction can be incubated under conditions that result in greater than 65% conversion of RNA to 5′-NMPs. In some embodiments, RNA is converted to 5′-NMPs at a rate of (or at least) 50 mM/hr, 100 mM/hr or 200 mM/hr.

In some embodiments, salt is added to a cell lysate, for example, to prevent enzyme aggregation. For example, sodium chloride, potassium chloride, sodium acetate, potassium acetate, or a combination thereof, can be added to a cell lysate. The concentration of salt in a cell lysate during an RNA depolymerization reaction can be 5 mM to 1 M. In some embodiments, the concentration of salt in a cell lysate during an RNA depolymerization reaction 5 mM, 10 mM, 15 mM, 20 mM, 25 mM, 50 mM, 100 mM, 150 mM, 200 mM, 250 mM, 500 mM, 750 mM, or 1 M. In some embodiments, the cell lysate comprises a mixture that includes 40-60 mM potassium phosphate, 1-5 mM MnCl₂, and/or 10-50 mM MgCl₂ (e.g., 20 mM MgCl₂).

In some embodiments, buffer is added to a cell lysate, for example, to achieve a particular pH value and/or salt concentration. Examples of buffers include, without limitation, phosphate buffer, Tris buffer, MOPS buffer, HEPES buffer, citrate buffer, acetate buffer, malate buffer, MES buffer, histidine buffer, PIPES buffer, bis-tris buffer, and ethanolamine buffer.

In some embodiments, Nuclease P1 is used to depolymerize the biomass. In some embodiments, the Nuclease P1 is filtered out of the reaction before subsequent steps.

Depolymerization of RNA can result in the production of 5′-NMPs, including 5′-AMP, 5′-UMP, 5′-CMP, and 5′-GMP. It should be understood that depolymerization of RNA does not result in any predetermined ratio of NMPs but will depend on the composition of the cellular RNA.

In some embodiments, PNPase is used to depolymerize the biomass. In some embodiments, the PNPase is inactivated or eliminated from the reaction before subsequent steps.

Depolymerization of RNA in the presence of phosphate can result in the production of 5′-NDPs, including 5′-ADP, 5′-UDP, 5′-CDP, and 5′-GDP. It should be understood that depolymerization of RNA does not result in any predetermined ratio of NDPs but will depend on the composition of the cellular RNA. As used herein, the use of PNPase for making NDPs requires the use of phosphate.

In some embodiments, 50-98% of the endogenous RNA in a cell upon lysis is converted to (depolymerized to) 5′-NMPs or 5′-NDPs. For example, 50-95%, 50-90%, 50-85%, 50-80%, 75-98%, 75-95%, 75-90%, 75-85% or 75-80% RNA is converted to (depolymerized to) 5′-NMPs. In some embodiments, 65-70% of the endogenous RNA in a cell upon lysis is converted to (depolymerized to) 5′-NMPs or 5′NDPs. Lower yields are also acceptable.

### Elimination or Inactivation of RNA Depolymerizing Enzyme

Following conversion of RNA from biomass (e.g., endogenous RNA) to its monomeric constituents (e.g., NMPs or NDPs) by endogenous and/or exogenous nucleases, there can remain in the reaction mixture or cell lysate several enzymes, including nucleases and phosphatases, which can have deleterious effects on RNA biosynthesis. For example, a nuclease used for depolymerization (e.g., Nuclease P1) can remain active following depolymerization of the biomass. As another example, cellular RNA sources contain numerous native phosphatases, many of which dephosphorylate NTPs, NDPs, and NMPs. Dephosphorylation of NMPs derived from cellular RNA following RNA depolymerization can result in the accumulation of non-phosphorylated nucleosides and result in a loss of usable NMP substrate, thus reducing synthetic RNA yield.

### Elimination or Inactivation of Undesired Enzymatic Activities

For reaction mixtures that include materials derived from cells, (e.g. cell lysate(s) or enzyme preparations obtained from cell lysate(s)), it may be advantageous to remove, eliminate, or inactivate undesired native enzymatic activities using any of the methods described herein. Undesired native enzymatic activities include, for example, phosphatases, nucleases, proteases, deaminases, oxidoreductases, and hydrolases. Dephosphorylation of NDPs or NTPs following RNA depolymerization to NMPs can result in futile energy cycles (energy cycles that produce a low yield of synthetic RNA) during which NMPs are phosphorylated to NDPs and NTPs and are in turn dephosphorylated to NMP or nucleoside starting point. Futile cycles reduce RNA product yield per unit energy input (e.g., polyphosphate, ATP, or other sources of high energy phosphate).

Numerous methods can be utilized to remove, eliminate, or inactivate undesired enzymatic activities. In some embodiments, undesired enzymatic activities are removed by removing genes encoding deleterious enzymes from the host genome. Enzymes deleterious to RNA biosynthesis, as provided herein, can be deleted from the host cell genome during engineering, provided the enzymes are not essential for host cell (e.g., bacterial cell) survival and/or growth. Deletion of enzymes or enzyme activities can be achieved, for example, by deleting or modifying in the host cell genome a gene encoding the enzyme. An enzyme is “essential for host cell survival” if a host cell cannot survive without expression and/or activity of a particular enzyme. Similarly, an enzyme is “essential for host cell growth” if a host cell cannot divide and/or grow without expression and/or activity of a particular enzyme.

If enzymes deleterious to the biosynthesis of RNA are essential for host cell survival and/or growth, other methods can be used. In some embodiments, the enzymatic activities are eliminated by heat inactivation. In some embodiments, the enzymatic activities are eliminated by a change in pH. In some embodiments, the enzymatic activities are eliminated by a change in salt concentration. In some embodiments, the enzymatic activities are eliminated by treatment with alcohol or another organic solvent. In some embodiments, the enzymatic activities are eliminated by detergent treatment. In some embodiments the enzymatic activities are eliminated through the use of chemical inhibitors. In some embodiments, the enzymatic activities are eliminated by physical separation, including, but not limited to, methods of filtration, precipitation, and capture, and/or chromatography. In some embodiments, the chromatography used is immobilized metal chromatography. In some embodiments, the capture method requires the enzyme to have a hexahistidine tag. A combination of any of the foregoing approaches can also be used.

In some embodiments, native enzymatic activity is removed via genetic modification, enzyme secretion from a cell, localization (e.g., periplasmic targeting), and/or protease targeting. In other embodiments, native enzymatic activity is inactivated via temperature, pH, salt, detergent, alcohol or other solvents, and/or chemical inhibitors. In yet other embodiments, native enzymatic activity is eliminated via physical separation, such as precipitation, filtration, capture, and/or chromatography.

Undesired (e.g., native) enzymatic activity(ies) may be removed using genetic, conditional, or separation approaches. In some embodiments, a genetic approach is used to remove undesired enzymatic activity. Thus, in some embodiments, cells are modified to reduce or remove undesired enzymatic activities. Examples of genetic approaches that may be used to reduce or remove undesired enzymatic activity(ies) include, but are not limited to, secretion, gene knockouts, and protease targeting. In some embodiments, a conditional approach is used to remove undesired enzymatic activity. Thus, in some embodiments, undesired enzymes exhibiting undesired activities remain in an enzyme preparation, a cell lysate, and/or a reaction mixture and are selectively inactivated. Examples of conditional approaches that may be used to reduce or remove undesired enzymatic activity include, but are not limited to, changes in temperature, pH, salt, detergent, alcohol or other solvents, and/or chemical inhibitors. In some embodiments, a separation/purification approach is used to remove undesired enzymatic activity. Thus, in some embodiments, undesired enzymes exhibiting undesired activities are physically separated from an enzyme preparation, a cell lysate, and/or a reaction mixture. Examples of separation approaches that may be used to reduce or eliminate undesired enzymatic activity include, but are not limited to, physical separation, such as filtration, precipitation, capture, and/or chromatography.

In various embodiments provided herein, enzymes prepared from cells or lysates of cells that express pathway enzymes are used in a reaction mixture for the production of NTP and/or RNA. In these cells or cell lysates, there are enzymes that may have deleterious effects on NTP and/or RNA production. Non-limiting examples of such enzymes include phosphatases, nucleases, proteases, deaminases, oxidoreductases, and/or hydrolases, such as those expressed by E. coli cells. Phosphatases remove phosphate groups (e.g., converting NMPs to nucleosides, converting NDPs to NMPs, or converting NTPs to NDPs), which reduce NTP production due to futile cycles of nucleotide phosphorylation/dephosphorylation. Nucleases cleave nucleic acids into monomers or oligomers, which lead to RNA product degradation (e.g., by RNase) and/or DNA template degradation (e.g., by DNase). Proteases cleave proteins into amino acids or peptides, which degrade pathway enzymes. Deaminases remove amino groups, which reduced NTP concentrations by conversion of pathway intermediates to non-useful substrates (e.g., xanthine and hypoxanthine) and can lead to mutations in RNA products (e.g., C to U). Hydrolases (e.g., nucleoside hydrolase or nucleotide hydrolase) cleave nucleosides or nucleotides into base and sugar moieties, which reduce NTP concentrations due to irreversible degradation of nucleotides. Oxidoreductases catalyze the transfer of electrons from one molecule (the oxidant) to another molecule (the reductant). Oxidation and/or reduction reactions can, for example, damage nucleobases in DNA and/or RNA, leading to errors in transcription and/or translation, or damage proteins or enzymes leading to loss of function.

Thus, it is advantageous in many embodiments to remove, eliminate, or inactivate these native enzymatic activities or other undesired enzymatic activities in an enzyme preparation, a cell lysate, and/or a reaction mixture.

Examples of enzymes that can be heat inactivated, deleted or physically removed from the genome of a host cell, include, without limitation, nucleases (e.g., RNase III, RNase I, RNase R, Nuclease P1, PNPase, RNase II, and RNase T), phosphatases (e.g., nucleoside monophosphatases, nucleoside diphosphatases, nucleoside triphosphatases), and other enzymes that depolymerize RNA or dephosphorylate nucleotides. Enzymes that depolymerize RNA include any enzyme that is able to cleave, partially hydrolyze, or completely hydrolyze a RNA molecule.

Examples of techniques to inactivate enzymes include conditional approaches. In some embodiments, an enzyme preparation, a cell lysate, and/or a reaction mixture includes an enzyme exhibiting undesired activity that is selectively inactivated. In some embodiments, an enzyme exhibiting undesired activity is selectively inactivated by exposing the enzyme to elimination conditions (e.g., high or low temperature, acidic or basic pH value, high salt or low salt, detergent, and/or organic solvent). In some embodiments, undesirable enzymatic activity is eliminated by precipitation or chromatography.

“Heat inactivation” refers to the process of heating a cell-free reaction mixture to a temperature sufficient to inactivate (or at least partially inactivate) endogenous nucleases, phosphatases, or other enzymes. Generally, the process of heat inactivation involves denaturation of (unfolding of) the deleterious enzyme. The temperature at which endogenous cellular proteins denature varies among organisms. In E. coli, for example, endogenous cellular enzymes generally denature at temperatures above 41° C. The denaturation temperature can be higher or lower than 41° C. for other organisms. Enzymes of a reaction mixture, as provide here, can be heat inactivated at a temperature of 55° C.-95° C., or higher. In some embodiments, enzymes of a reaction mixture can be heat inactivated at a temperature of 55-90° C., 55-80° C., 55-70° C. 55-60° C., 60-95° C., 60-90° C., 60-80° C., 60-70° C., 70-95° C., 70-90° C. or 70-80° C. For example, enzymes in a cell-free reaction mixture can be heat inactivated at a temperature of 55° C., 60° C., 65° C., 70° C., 75° C., 80° C. 85° C., 90° C., or 95° C. In some embodiments, enzymes of a cell-free reaction mixture can be heat inactivated at a temperature of 55-95° C. In some embodiments, enzymes of a cell-free reaction mixture can be heat inactivated at a temperature of 70° C. In some embodiments, enzymes of a cell-free reaction mixture can be heat inactivated at a temperature of 60° C. It can also be possible to introduce chemical inhibitors of deleterious enzymes. Such inhibitors can include, but are not limited to, sodium orthovanadate (inhibitor of protein phosphotyrosyl phosphatases), sodium fluoride (inhibitor of phosphoseryl and phosphothreonyl phosphatases), sodium pyrophosphate (phosphatase inhibitor), sodium phosphate, and/or potassium phosphate.

The period of time during which a cell-free reaction mixture is incubated at elevated temperatures to achieve heat inactivation of undesired enzymes can vary, depending, for example, on the volume of the cell-free reaction mixture and the organism from which biomass was prepared. In some embodiments, a cell-free reaction mixture is incubated at a temperature of 55° C.-99° C. for 0.5 minutes (min) to 24 hours (hr). For example, a cell-free reaction mixture can be incubated at a temperature of 55° C.-99° C. for 0.5 min, 1 min, 2 min, 4 min, 5 min, 10 min, 15 min, 30 min, 45 min, or 1 hr. In some embodiments, a cell-free reaction mixture is incubated at a temperature of 55° C.-99° C. for 30 minutes, 1, 2, 3, 4, 5, 6, 7, 8, 9, 10, 11, 12, 13, 14, 15, 16, 17, 18, 19, 20, 24, 36, 42, or 48 hr.

In some embodiments, enzymes are heat inactivated at a temperature of 60-80° C. for 10-20 min. In some embodiments, enzymes are heat inactivated at a temperature of 70° C. for 15 min.

In some embodiments, enzymes that depolymerize endogenous RNA comprise one or more modifications (e.g., mutations) that render the enzymes more sensitive to heat. These enzymes are referred to as “heat-sensitive enzymes.” Heat-sensitive enzymes denature and become inactivated at temperatures lower than that of their wild-type counterparts, and/or the period of time required to reduce the activity of the heat-sensitive enzymes is shorter than that of their wild-type counterparts.

It should be understood that enzymes that are heat inactivated can, in some instances, retain some degree of activity. For example, the activity level of a heat-inactivated enzyme can be less than 50% of the activity level of the same enzyme that has not been heat inactivated. In some embodiments, the activity level of a heat-inactivated enzyme is less than 40%, less than 30%, less than 20%, less than 10%, less than 5%, less than 1%, or less than 0.1% of the activity level of the same enzyme that has not been heat inactivated.

Thus, an enzyme's activity can be completely eliminated or reduced. An enzyme is considered completely inactive if the denatured (heat inactivated) form of the enzyme no longer catalyzes a reaction catalyzed by the enzyme in its native form. A heat-inactivated, denatured enzyme is considered “inactivated” when activity of the heat-inactivated enzyme is reduced by at least 50% relative to activity of the enzyme that is not heated (e.g., in its native environment). In some embodiments, activity of a heat-inactivated enzyme is reduced by 50-100% relative to the activity of the enzyme that is not heated. For example, activity of a heat-inactivated enzyme is reduced by 50-90%, 50-85%, 50-80%, 50-75%, 50-70%, 50-65%, 50-60%, or 50-55% relative to activity of the enzyme that is not heated. In some embodiments, the activity of a heat-inactivated enzyme is reduced by 25%, 30%, 35%, 40%, 45%, 50%, 55%, 60%, 65%, 70%, 75%, 80%, 85%, 90%, 95%, 98%, 99%, or 100% relative to the activity of the enzyme that is not heated.

In some embodiments, a reaction mixture is exposed to an acid or base (change in pH) that temporarily or irreversibly inactivates an enzyme exhibiting undesired activity. “Acid or base inactivation” refers to the process of adjusting a reaction mixture to a pH sufficient to inactivate (or at least partially inactivate) undesired enzyme(s). Generally, the process of acid or base inactivation involves denaturation of (unfolding of) the enzyme(s). The pH at which enzymes denature varies among organisms. In E. coli, for example, native enzymes generally denature at pH above 7.5 or below 6.5. The denaturation pH can be higher or lower than the denaturation pH for other organisms. Enzymes of a reaction mixture, as provide herein, can be base inactivated at a pH of 7.5-14, or higher. In some embodiments, enzymes of a cell-free reaction mixture are base inactivated at a pH of 8-14, 8.5-14, 9-14, 9.5-14, 10-14, 10.5-14, 11-14, 11.5-14, 12-14, 12.5-14, 13-14, or 13.5-14. In some embodiments, enzymes of a cell-free reaction mixture are base inactivated at a pH of 7.5-13.5, 7.5-13, 7.5-12.5, 7.5-12, 7.5-11.5, 7.5-11, 7.5-10.5, 7.5-10, 7.5-9.5, 7.5-9, 7.5-8.5, or 7.5-8. For example, enzymes of a cell-free reaction mixture can be base inactivated at a pH of approximately 7.5, 8, 8.5, 9, 9.5, 10, 10.5, 11, 11.5, 12, 12.5, 13, 13.5, or 14. Enzymes of a cell-free reaction mixture, as provide herein, can be acid inactivated at a pH of 6.5-0, or lower. In some embodiments, enzymes of a cell-free reaction mixture are acid inactivated at a pH of 6.5-0.5, 6.5-1, 6.5-1.5, 6.5-2, 6.5-2.5, 6.5-3, 6.5-3.5, 6.5-4, 6.5-4.5, 6.5-5, or 6.5-6. In some embodiments, enzymes of a cell-free reaction mixture are acid inactivated at a pH of 6-0, 5.5-0, 5-0, 4.5-0, 4-0, 3.5-0, 3-0, 2.5-0, 2-0, 1.5-0, 1-0, or 0.5-0. For example, enzymes of a cell-free reaction mixture can be acid inactivated at a pH of approximately 6.5, 6, 5.5, 5, 4.5, 4, 3.5, 3, 2.5, 2, 1.5, 1, 0.5, or 0.

In some embodiments, a cell-free reaction mixture is exposed to a high salt or low salt (change in salt concentration) that temporarily or irreversibly inactivates an enzyme exhibiting undesired activity. “Salt inactivation” refers to the process of adjusting an enzyme preparation, a cell lysate, and/or a cell-free reaction mixture to a salt concentration sufficient to inactivate (or partially inactivate) an enzyme. Generally, the process of salt inactivation involves denaturation of (unfolding of) the enzyme. The salt concentration at which enzymes denature varies among organisms. In E. coli, for example, native enzymes generally denature at a salt concentration above 600 mM. The denaturation salt concentration can be higher or lower than the denaturation salt concentration for other organisms. Salts are combinations of anions and cations. Non-limiting examples of cations that can be used for salt inactivation of undesired enzyme activities in a cell-free reaction mixture as set forth herein include lithium, sodium, potassium, magnesium, calcium and ammonium. Non-limiting examples of anions that can be used for salt inactivation of undesired enzyme activities in a cell-free reaction mixture as set forth herein include acetate, chloride, sulfate, and phosphate. Enzymes of a cell-free reaction mixture, as provided herein, can be salt inactivated at a salt concentration of 600-1000 mM, or higher. In some embodiments, enzymes of an enzyme preparation, a cell lysate, and/or a cell-free reaction mixture can be salt inactivated at a salt concentration of 700-1000 mM, 750-1000 mM, 800-1000 mM, 850-1000 mM, 900-1000 mM, 950-1000 mM. In some embodiments, enzymes of an enzyme preparation, a cell lysate, and/or a cell-free reaction mixture can be salt inactivated at a salt concentration of 600-950 mM, 600-900 mM, 600-850 mM, 600-800 mM, 600-750 mM, 600-700 mM, or 600-650 mM. For example, enzymes of an enzyme preparation, a cell lysate, and/or a cell-free reaction mixture may be salt inactivated at a salt concentration of approximately 600 mM, 650 mM, 700 mM, 750 mM, 800 mM, 850 mM, 900 mM, 950 mM, or 1000 mM. Enzymes of an enzyme preparation, a cell lysate, and/or a cell-free reaction mixture, as provided herein, may be salt inactivated at a salt concentration of 400-1 mM, or lower. In some embodiments, enzymes of an enzyme preparation, a cell lysate, and/or a cell-free reaction mixture can be salt inactivated at a salt concentration of 350-1 mM, 300-1 mM, 250-1 mM, 200-1 mM, 150-1 mM, 100-1 mM, or 50-1 mM. In some embodiments, enzymes of an enzyme preparation, a cell lysate, and/or a cell-free reaction mixture be salt inactivated at a salt concentration of 400-50 mM, 400-100 mM, 400-150 mM, 400-200 mM, 400-250 mM, 400-300 mM, or 400-350 mM. For example, enzymes of an enzyme preparation, a cell lysate, and/or a cell-free reaction mixture may be salt inactivated at a salt concentration of approximately 400 mM, 350 mM, 300 mM, 250 mM, 200 mM, 150 mM, 100 mM, 50 mM, or 1 mM.

In some embodiments, an organic solvent is added to an enzyme preparation, a cell lysate, and/or a reaction mixture to inactivate an enzyme exhibiting undesired activity. Non-limiting examples of organic solvents include ethanol, methanol, ether, dioxane, acetone, methyl ethyl ketone, acetonitrile, dimethyl sulfoxide, and toluene.

In some embodiments, a detergent is added to an enzyme preparation, a cell lysate, and/or a reaction mixture to inactivate an enzyme exhibiting undesired activity. Non-limiting examples of detergents include sodium dodecyl sulfate (SDS), ethyl trimethylammonium bromide (ETMAB), lauryl trimethyl ammonium bromide (LTAB), and lauryl trimethylammonium chloride (LTAC).

In some embodiments, a chemical inhibitor is added to an enzyme preparation, a cell lysate, and/or a reaction mixture to inactivate an enzyme exhibiting undesired activity. Non-limiting examples of chemical inhibitors include sodium orthovanadate (inhibitor of protein phosphotyrosyl phosphatases), sodium fluoride (inhibitor of phosphoseryl and phosphothreonyl phosphatases), sodium pyrophosphate (phosphatase inhibitor), sodium phosphate, and/or potassium phosphate. In some embodiments, chemical inhibitors are selected from a chemical inhibitor library.

For any of the conditional approaches used herein, it should be understood that any of the pathway enzymes present in the cell lysate or cell-free reaction mixture may also be exposed to the elimination conditions (e.g., high or low temperature, acidic or basic pH value, high salt or low salt, detergent and/or organic solvent). Thus, in some embodiments, the pathway enzymes (e.g., polyphosphate kinase, NMP kinase, NDP kinase, and/or polymerase) can withstand elimination conditions. An enzyme is considered to withstand elimination conditions if the enzyme, following exposure to the elimination conditions, retains at least 10% (e.g., at least 20%, at least 30%, at least 40%, at least 50%, at least 60%, at least 70%, at least 80%, or at least 90%) of its enzymatic activity (relative to enzymatic activity prior to exposure to the inactivation condition).

For example, when native enzymes of an enzyme preparation, a cell lysate, and/or a cell-free reaction mixture are heat-inactivated (e.g., exposed to a temperature of at least 55° C., or 55-95° C., for at least 30 seconds or 30 seconds-60 min), the pathway enzymes can be thermostable enzymes. Thus, in some embodiments, at least one of a polyphosphate kinase, NMP kinase, NDP kinase, nucleoside kinase, phosphoribosyltransferase, nucleoside phosphorylase, ribokinase, phosphopentomutase, and polymerase is a thermostable variant thereof Δn enzyme (e.g., kinase or polymerase) is considered thermostable if the enzyme (a) retains activity after temporary exposure to high temperatures that denature native enzymes (i.e. 42° C.) or (b) functions at a high rate after temporary exposure to a medium to high temperature where native enzymes function at low rates. Thermostable enzymes are known, and non-limiting examples of thermostable enzymes for use are provided herein. Other non-limiting examples of pathway enzymes that can withstand elimination conditions are also provided herein. In some embodiments, a native enzyme exhibiting undesired activity is physically removed from a reaction mixture. In some embodiments, an enzyme exhibiting undesired activity is precipitated out of a cell-free reaction mixture. In some embodiments, an enzyme exhibiting undesired activity is filtered (e.g., based on size) from a reaction mixture. In some embodiments, an enzyme exhibiting undesired activity is removed from a reaction mixture via capture and/or chromatography (e.g., by differential affinity to a stationary phase). In some embodiments, an enzyme exhibiting undesired activity is removed from a reaction mixture via affinity chromatography. Examples of affinity chromatography include, but are not limited to, Protein A chromatography, Protein G chromatography, metal binding chromatography (e.g., nickel chromatography), lectin chromatography, and GST chromatography. In some embodiments, an enzyme exhibiting undesired activity is removed from a reaction mixture via ion exchange chromatography. Examples of anion exchange chromatography (AEX) include, but are not limited to, diethylaminoethyl (DEAE) chromatography, quaternary aminoethyl (QAE) chromatography, and quaternary amine (Q) chromatography. Examples of cation exchange chromatography include, but are not limited to, carboxymethyl (CM) chromatography, sulfoethyl (SE) chromatography, sulfopropyl (SP) chromatography, phosphate (P) chromatography, and sulfonate (S) chromatography. In some embodiments, an enzyme exhibiting undesired activity is removed from a reaction mixture via hydrophobic interaction chromatography (HIC). Examples of hydrophobic interaction chromatography include, but are not limited to, Phenyl Sepharose chromatography, Butyl Sepharose chromatography, Octyl Sepharose chromatography, Capto Phenyl chromatography, Toyopearl Butyl chromatography, Toyopearl Phenyl chromatography, Toyopearl Hexyl chromatography, Toyopearl Ether chromatography, and Toyopearl PPG chromatography. Any of the chemistries detailed above could be alternatively be used to immobilize or capture pathway enzymes.

Nuclease P1 from Penicillium citrinum, a zinc dependent-enzyme, hydrolyzes both 3′-5′-phosphodiester bonds in RNA and heat denatured DNA and 3′-phosphomonoester bonds in mono- and oligonucleotides terminated by 3′-phosphate without base specificity. Nuclease P1 is capable of hydrolyzing single-stranded DNA and RNA completely to the level of ribonucleoside 5′-monophosphates.

E. coli RNase I localizes to the periplasmic space in intact bacterial cells and catalyzes depolymerization of a wide range of RNA molecules, including rRNA, mRNA, and tRNA. Under physiological conditions the periplasmic localization of this enzyme means that the enzyme has little impact on RNA stability within the cell; however, mixing of the periplasm and cytoplasm in bacterial cell lysates permits RNase I access to cellular RNA. The presence of RNase I in a cell lysate can reduce the yield of synthetic RNA through RNA degradation. Neither RNase I nor the gene encoding RNase I, rna, is essential for cell viability, thus, in some embodiments, rna is deleted or mutated in engineered host cells. In other embodiments, RNase I in the reaction mixture is heat inactivated following depolymerization of endogenous RNA.

E. coli RNase R and RNase T catalyze the depolymerization of dsRNA, rRNA, tRNA, and mRNA, as well as small unstructured RNA molecules. Neither the enzymes nor the genes encoding the enzymes, rnr and rnt, respectively, are essential for bacterial cell viability, thus, in some embodiments, rnr and/or rnt are deleted or mutated in engineered host cells (e.g., E. coli host cells). In other embodiments, RNase R and/or RNase T in a cell-free reaction mixture can be heat inactivated following the depolymerization of endogenous RNA.

E. coli RNase E and PNPase are components of the degradasome, which is responsible for mRNA turnover in cells. RNase E is thought to function together with PNPase and RNase II to turn over cellular mRNA pools. Disruption of the gene encoding RNase E, rne, is lethal in E. coli. Thus, in some embodiments, RNase E in a cell-free reaction mixture can be heat inactivated following depolymerization of endogenous RNA. Neither PNPase nor the gene encoding PNPase, pnp, is essential for cell viability, thus, in some embodiments, pnp can be deleted or mutated in engineered host cells (e.g., E. coli host cells). In other embodiments, PNPase in the reaction mixture is heat inactivated following depolymerization of endogenous RNA.

E. coli RNase II depolymerizes both mRNA and tRNA in a 3′ to 5′ direction. Neither RNase II nor the gene encoding RNase II, rnb, is essential for cell viability, thus, in some embodiments, rnb is deleted or mutated in engineered host cells. In other embodiments, RNase II in the reaction mixture is heat inactivated following depolymerization of endogenous RNA.

While neither pnp nor rnb is essential to host cell survival, disruption of both simultaneously can be lethal. Thus, in some embodiments, both PNPase and RNase II are heat inactivated.

### Phosphorylation of Nucleoside Monophosphates or Nucleoside Diphosphates to Nucleoside Triphosphates

Following conversion of cellular RNA to its component monomers, and following elimination or inactivation of the nuclease(s) that depolymerize RNA and undesired enzymatic activities, the resulting nucleoside monophosphates (NMPs) or nucleoside diphosphates (NDPs) are phosphorylated before they are polymerized to form a desired synthetic RNA, such as a single-stranded mRNA. This process is energy dependent and thus requires an energy source, typically a high-energy phosphate source, such as, for example, phosphoenolpyruvate, ATP, or polyphosphate.

In some embodiments, the energy source is ATP that is directly added to a cell lysate. In other embodiments, the energy source is provided using an ATP regeneration system. For example, polyphosphate and polyphosphate kinase can be used to produce ATP. Other examples included using acetyl-phosphate and acetate kinase to produce ATP; phospho-creatine and creatine kinase to produce ATP; and phosphoenolpyruvate and pyruvate kinase to produce ATP. Other ATP (or other energy) regeneration systems can be used. In some embodiments, at least one component of the energy source is added to a cell lysate, cell lysate mixture, or cell-free reaction mixture. A “component” of an energy source includes substrate(s) and enzyme(s) required to produce energy (e.g., ATP). Non-limiting examples of these components include polyphosphate with polyphosphate kinase, acetyl-phosphate with acetate kinase, phospho-creatine with creatine kinase, and phosphoenolpyruvate with pyruvate kinase. In some embodiments, the polyphosphate kinase is Deinococcus geothermalis polyphosphate kinase 2 (DgPPK2). In some embodiments, the polyphosphate kinase is the kinase represented in SEQ ID NO: 1.

A kinase is an enzyme that catalyzes transfer of phosphate groups from high-energy, phosphate-donating molecules, such as ATP, to specific substrates/molecules. This process is referred to as phosphorylation, where a substrate gains a phosphate group donated from a high-energy ATP molecule. This transesterification produces a phosphorylated substrate and ADP. Kinases of the present disclosure, in some embodiments, convert NMPs to NDPs and NDPs to NTPs. Both nucleotide-specific (AMP, GMP, CMP, UMP) and panspecific (NDP) transfer enzymes are contemplated for use in the invention.

In some embodiments, a kinase is a nucleoside monophosphate kinase, which catalyzes the transfer of a high-energy phosphate from ATP to an NMP, resulting in ADP and NDP. In some embodiments, a cell lysate comprises one or more (or all) of the following four nucleoside monophosphate kinases: uridylate kinase, cytidylate kinase, guanylate kinase and adenylate kinase. Exemplary nucleoside monophosphate kinases are listed in Tables 2-5. Thermostable variants of the enzymes are encompassed by the present disclosure. In some embodiments, one or more of the nucleoside monophosphate kinases is thermostable. In a preferred embodiment, all of the nucleoside monophosphate kinases are thermostable. In some embodiments, the thermostable kinases have their undesirable activities heat-inactivated prior to use in any NTP or RNA production reactions. In some embodiments, the uridylate kinase is from or derived from Pyrococcus furiosus. In some embodiments, the uridylate kinase is the kinase represented in SEQ ID NO: 14. In some embodiments, the cytidylate kinase is from or derived from Thermus thermophiles. In some embodiments, the cytidylate kinase is the kinase represented in SEQ ID NO: 13. In some embodiments, the guanylate kinase is from or derived from Thermotoga maritima. In some embodiments, the guanylate kinase is the kinase represented in SEQ ID NO: 15. In some embodiments, the adenylate kinase is from Thermus thermophilus. In some embodiments, the adenylate kinase is the kinase represented in SEQ ID NO: 12.

TABLE 2
Examples of AMP kinase enzymes

| | | | | |
|---|---|---|---|---|
| | | | Sequence | |
| | | | Identifi- | |
| En- | | GenBank # | cation | Refer- |
| zyme | Organism | UniProt # | Number | ence |

Thermophiles

| | | | | |
|---|---|---|---|---|
| Adk | Thermus thermophilus | Q72I25 | SEQ ID | 25, 26 |
| | | | NO: 12 | |
| Adk | Pyrococcus furiosus | Q8U207 | | 27 |

Solvent-tolerant organisms

| | | | |
|---|---|---|---|
| Adk | Pseudomonas putida | AFO48764.1 | 42 |
| | DOT-T1E | I7CAA9 | |
| Adk | Escherichia coli K-12 | BAE76253.1 | 44 |
| | W3110 | P69441 | |
| Adk1 | Aspergillus niger CBS | CAK45139.1 | 45 |
| | 513.88 | A2QPN9 | |
| Adk1 | Saccharomyces cerevisiae | AAC33143.1 | 46 |
| | ATCC 204508 / S288c | P07170 | |
| Adk | Clostridium | AAK81051.1 | 43 |
| | acetobutylicum | Q97EJ9 | |
| | ATCC 824 | | |
| Adk | Halobacterium salinarum | AAG19963.1 | 32 |
| | ATCC 700922 | Q9HPA7 | |

Acidophiles

| | | |
|---|---|---|
| Adk | Acidithiobacillus | WP_024894015.1 |
| | thiooxidans | |
| Adk | Acidithiobacillus | WP_064218420.1 |
| | ferrooxidans | |
| Adk | Acetobacter aceti | WP_077811596.1 |
| Adk | Bacillus acidicola | WP_066267988.1 |
| Adk | Sulfolobus solfataricus | WP_009991241.1 |

Alkaliphiles

| | | |
|---|---|---|
| Adk | Thioalkalivibrio | WP_019570706.1 |
| Adk | Amphibacillus xylanus | WP_015008883.1 |

Psychrophiles

| | | | |
|---|---|---|---|
| Adk | Colwellia psychrerythraea | WP_033093471.1 | 28 |
| | (Vibrio psychroerythus) | Q47XA8 | |
| Adk | Psychromonas ingrahamii | WP_011769361 | |
| | | A1STI3 | |
| Adk | Pseudoalteromonas | CAI86283 | 29 |
| | haloplanktis | Q3IKQ1 | |
| Adk | Psychrobacter arcticus | WP_011280822 | 30 |
| Adk | Pseudomonas syringae | WP_004406317.1 | 31 |
| | | Q4ZWV2 | |

Halophiles

| | | | |
|---|---|---|---|
| Adk | Halobacterium halobium | WP_010903261.1 | 32 |
| | | Q9HPA7 | |

TABLE 3
Examples of CMP kinase enzymes

| | | | | |
|---|---|---|---|---|
| | | | Sequence | |
| | | | Identifi- | |
| En- | | GenBank # | cation | Refer- |
| zyme | Organism | UniProt # | Number | ence |

Thermophiles

| | | | | |
|---|---|---|---|---|
| Cmk | Thermus thermophilus | Q5SL35 | SEQ ID | 33 |
| | | | NO: 13 | |
| Cmk | Pyrococcus furiosus | Q8U2L4 | | 27 |

Solvent-tolerant organisms

| | | | |
|---|---|---|---|
| Cmk | Pseudomonas putida | AFO48857.1 | 42 |
| | DOT-T1E | I7BXE2 | |
| Cmk | Escherichia coli K-12 | AAC73996.1 | 47 |
| | MG1655 | P0A6I0 | |
| Cmk | Clostridium | AAK79812.1 | 43 |
| | acetobutylicum | Q97I08 | |
| | ATCC 824 | | |
| Cmk | Halobacterium salinarum | AAG19965.1 | 34 |
| | ATCC 700922 | Q9HPA5 | |

Acidophiles

| | | |
|---|---|---|
| Cmk | Bacillus acidicola | WP_066270173 |
| Cmk | Acetobacter aceti | WP_010667744 |
| Cmk | Acidithiobacillus | WP_024892761.1 |
| | thiooxidans | |
| Cmk | Acidithiobacillus | WP_064220349.1 |
| | ferrooxidans | |
| Cmk | Metallosphaera sedula | WP_011921264.1 |

Alkaliphiles

| | | |
|---|---|---|
| Cmk | Amphibacillus xylanus | WP_015009966.1 |
| Cmk | Thioalkalivibrio | WP_077278466.1 |
| | denitrificans | |

Psychrophiles

| | | | |
|---|---|---|---|
| Cmk | Colwellia psychrerythraea | WP_011043148.1 | 28 |
| | (Vibrio psychroerythus) | Q482G4 | |
| Cmk | Pseudoalteromonas | CAI86499.1 | 29 |
| | haloplanktis | Q3ILA1 | |
| Cmk | Psychrobacter arcticus | AAZ19343.1 | 30 |
| | | Q4FRL5 | |
| Cmk | Psychromonas ingrahamii | ABM04716 | |
| | | A1SZ01 | |
| Cmk | Pseudomonas syringae | YP_236713 | 31 |
| | | Q4ZQ97 | |

Halophiles

| | | | |
|---|---|---|---|
| Cmk | Halobacterium salinarum | Q9HPA5 | 34 |

TABLE 4
Examples of UMP kinase enzymes

| | | | | |
|---|---|---|---|---|
| | | | Se- | |
| | | | quence | |
| | | | Identifi- | |
| | | GenBank # | cation | Refer- |
| Enzyme | Organism | UniProt # | Number | ence |

Thermophiles

| | | | | |
|---|---|---|---|---|
| PyrH | Pyrococcus furiosus | Q8U122 | SEQ ID | 35, 36 |
| | | | NO: 14 | |
| PyrH | Thermus | P43891 | | 33 |
| | thermophilus | | | |

Solvent-tolerant organisms

| | | | |
|---|---|---|---|
| PyrH | Pseudomonas putida | AFO48412.1 | 42 |
| | DOT-T1E | I7BW46 | |
| PyrH | Escherichia coli | CAA55388.1 | 48 |
| | K-12 MG1655 | P0A7E9 | |
| An13g00440 | Aspergillus niger | CAK41445.1 | 45 |
| | CBS 513.88 | A2R195 | |
| URA6 | Saccharomyces | AAA35194.1 | 49 |
| | cerevisiae ATCC | P15700 | |
| | 204508 / S288c | | |
| PyrH | Clostridium | AAK79754.1 | 43 |
| | acetobutylicum | Q97I64 | |
| | ATCC 824 | | |
| PyrH | Halobacterium | AAG20182.1 | 34 |
| | salinarum ATCC | Q9HNN8 | |
| | 700922 | | |

Acidophiles

| | | | |
|---|---|---|---|
| PyrH | Picrophilus torridus | WP_048059653 | |
| PyrH | Metallosphaera | WP_012021705 | |
| | sedula | | |
| PyrH | Ferroplasma | WP_009886950.1 | |
| PyrH | Thermoplasma | WP_010900913 | |
| | acidophilum | | |
| PyrH | Sulfolobus | WP_009992427 | 37 |
| | solfataricus | | |
| PyrH | Acetobacter aceti | WP_042788648 | |

Alkaliphiles

| | | |
|---|---|---|
| PyrH | Thioalkalivibrio sp. | WP_081759172.1 |
| | HK1 | |
| PyrH | Amphibacillus | WP_015010200.1 |
| | xylanus | |

Psychrophiles

| | | | |
|---|---|---|---|
| PyrH | Colwellia | WP_011042391.1 | 28 |
| | psychrerythraea | Q485G8 | |
| | (Vibrio | | |
| | psychroerythus) | | |
| PyrH | Pseudoalteromonas | CR954246.1 | 29 |
| | haloplanktis | Q3IIX6 | |
| PyrH | Psychrobacter | AAZ19383.1 | 30 |
| | arcticus | Q4FRH5 | |
| PyrH | Psychromonas | ABM04676.1 | |
| | ingrahamii | A1SYW1 | |
| PyrH | Pseudomonas | YP_234434 | 31 |
| | syringae | Q4ZWS6 | |

Halophiles

| | | | |
|---|---|---|---|
| PyrH | Halobacterium | WP_010903483.1 | 34 |
| | salinarum | Q9HNN8 | |

TABLE 5
Examples of GMP kinase enzymes

| | | | | |
|---|---|---|---|---|
| | | | Se- | |
| | | | quence | |
| | | | Identifi- | |
| | | GenBank # | cation | Refer- |
| Enzyme | Organism | UniProt # | Number | ence |

Thermophiles

| | | | | |
|---|---|---|---|---|
| Gmk | Thermotoga maritima | Q9X215 | SEQ ID | 38 |
| | | | NO: 15 | |
| Gmk | Thermus thermophilus | Q5SI18 | | 33 |

Solvent-tolerant organisms

| | | | |
|---|---|---|---|
| Gmk | Pseudomonas putida | AFO49847.1 | 42 |
| | DOT-T1E | I7C087 | |
| Gmk | Escherichia coli K-12 | AAB88711.1 | 50 |
| | | P60546 | |
| An08g00300 | Aspergillus niger | CAK45182.1 | 45 |
| | CBS 513.88 | A2QPV2 | |
| GUK1 | Saccharomyces | AAA34657.1 | 51 |
| | cerevisiae ATCC | P15454 | |
| | 204508 / S288c | | |
| Gmk | Clostridium | AAK79684.1 | 43 |
| | acetobutylicum | Q97ID0 | |
| | ATCC 824 | | |

Acidophiles

| | | |
|---|---|---|
| Gmk | Acidithiobacillus | WP_064219869.1 |
| | ferrooxidans | |
| Gmk | Acidithiobacillus | WP_010637919.1 |
| | thiooxidans | |
| Gmk | Bacillus acidicola | WP_066264774.1 |
| Gmk | Acetobacter aceti | WP_018308252.1 |

Alkaliphiles

| | | |
|---|---|---|
| Gmk | Amphibacillus | WP_015010280.1 |
| | xylanus | |
| Gmk | Thioalkalivibrio | WP_018953989.1 |
| | sulfidiphilus | |

Psychrophiles

| | | | |
|---|---|---|---|
| Gmk | Colwellia | AAZ24463 | 28 |
| | psychrerythraea | Q47UB3 | |
| | (Vibrio | | |
| | psychroerythus) | | |
| Gmk | Pseudoalteromonas | Q3IJH8 | 29 |
| | haloplanktis | | |
| Gmk | Psychrobacter | WP_011280984.1 | 30 |
| | arcticus | Q4FQY7 | |
| Gmk | Psychromonas | ABM05306 | |
| | ingrahamii | A1T0P1 | |
| Gmk | Pseudomonas | WP_003392601.1 | 31 |
| | syringae | Q4ZZY8 | |

In some embodiments, a kinase is a nucleoside diphosphate kinase, which transfers a phosphoryl group to NDP, resulting in NTP. The donor of the phosphoryl group can be, without limitation, ATP, polyphosphate polymer, or phosphoenolpyruvate. Non-limiting examples of kinases that convert NDP to NTP include nucleoside diphosphate kinase, polyphosphate kinase, and pyruvate kinase. Thermostable variants of the foregoing enzymes are encompassed by the present disclosure. In some embodiments, the NDP kinase(s) is/are obtained from Aquifex aeolicus.

Phosphorylation of NMPs to NTPs occurs, in some embodiments, through a polyphosphate-dependent kinase pathway, where high-energy phosphate is transferred from polyphosphate to ADP via a polyphosphate kinase (PPK). In some embodiments, the polyphosphate kinase belongs to the polyphosphate kinase 1 (PPK1) family, which transfers high-energy phosphate from polyphosphate to ADP to form ATP. This ATP is subsequently used by NMP kinases to convert NMPs to their cognate ribonucleotide diphosphates (NDPs). NMP kinases include, but are not limited to, AMP kinase, UMP kinase, GMP kinase, and/or CMP kinase. Furthermore, ATP is subsequently used by nucleotide diphosphate kinase to convert NDPs to NTPs. In some embodiments, polyphosphate kinases used in the methods disclosed herein are polyphosphate kinase 2 (PPK2) family kinases. In some particular embodiments, the polyphosphate kinase belongs to a Class I PPK2 family, which transfers high-energy phosphate from polyphosphate to NDPs to form NTPs. ATP produced by the system is used as a high-energy phosphate donor to convert NMPs to NDPs. In some particular embodiments, the polyphosphate kinase belongs to a Class III PPK2 family, which transfers high-energy phosphate from polyphosphate to NMPs and NDPs to form NTPs. In some embodiments, Class III PPK2 is used alone to produce NTPs from NMPs. In other embodiments. Class III PPK2 is used in combination with other kinases. Class III PPK2 produces ATP from ADP, AMP, and polyphosphate, which is subsequently used by NMP and NDP kinases to convert NMPs to NTPs. Exemplary polyphosphate kinases are listed in Table 6.

TABLE 6
Polyphosphate Kinases

| | | | | |
|---|---|---|---|---|
| | | | Sequence | |
| | | | Identifi- | |
| En- | | GenBank # | cation | |
| zyme | Organism | UniProt # | Number | Reference |

Thermophiles

| | | | | |
|---|---|---|---|---|
| PPK2 | Deinococcus | WP_011531362.1 | SEQ ID | 20 |
| | geothermalis DSM | | NO: 1 | |
| | 11300 | | | |
| PPK2 | Meiothermus ruber | ADD29239.1 | SEQ ID | 20 |
| | DSM 1279 | | NO: 2 | |
| PPK2 | Meiothermus silvanus | WP_013159015.1 | SEQ ID | 20 |
| | DSM 9946 | | NO: 3 | |
| PPK2 | Thermosynechococcus | NP_682498.1 | SEQ ID | 20 |
| | elongatus BP-1 | | NO: 4 | |
| PPK2 | Anaerolinea | WP_013558940 | SEQ ID | |
| | thermophila UNI-1 | | NO: 5 | |
| PPK2 | Caldilinea aerophila | WP_014433181 | SEQ ID | |
| | DSM 14535 | | NO: 6 | |
| PPK2 | Chlorobaculum | NP_661973.1 | SEQ ID | |
| | tepidum TLS | | NO: 7 | |
| PPK2 | Oceanithermus | WP_013458618 | SEQ ID | |
| | profundus DSM 14977 | | NO: 8 | |
| PPK2 | Roseiflexus | WP_012120763 | SEQ ID | |
| | castenholzii DSM | | NO: 9 | |
| | 13941 | | | |
| PPK2 | Roseiflexus sp. RS-1 | WP_011956376 | SEQ ID | |
| | | | NO: 10 | |
| PPK2 | Truepera radiovictrix | WP_013178933 | SEQ ID | |
| | DSM 17093 | | NO: 11 | |

Solvent-tolerant organisms

| | | | |
|---|---|---|---|
| PPK1 | Pseudomonas putida | AFO50238.1 | 42 |
| | DOT-T1E | I7BEV8 | |
| PPK1 | Escherichia coli K-12 | AAC75554.1 | |
| | | P0A7B1 | |
| PPK1 | Clostridium | NP_347259.1 | 43 |
| | acetobutylicum ATCC | Q97LE0 | |
| | 824 | | |

Acidophiles

| | | |
|---|---|---|
| PPK1 | Thermosynechococcus | WP_011056068 |
| | elongatus | |
| PPK1 | Acidithiobacillus | WP_064219446 |
| | ferrooxidans | |
| PPK1 | Acidithiobacillus | WP_031572361 |
| | thiooxidans | |
| PPK1 | Bacillus acidicola | WP_066264350 |
| PPK1 | Acetobacter aceti | GAN58028 |
| PPK2 | Acetobacter aceti | WP_077811826.1 |
| PPK2 | Acidithiobacillus | WP_051690689.1 |
| | thiooxidans | |
| PPK2 | Acidithiobacillus | WP_064219816.1 |
| | ferrooxidans | |

Alkaliphiles

| | | |
|---|---|---|
| PPK1 | Thioalkalivibrio | WP_077277945.1 |
| | denitrificans | |

Psychrophiles

| | | |
|---|---|---|
| PPK1 | Psychromonas | WP_041766473.1 |
| | ingrahamii | |
| PPK2 | Psychrobacter arcticus | WP_083756052.1 |
| PPK2 | Psychroserpens | WP_033960485.1 |
| | jangbogonensis | |
| PPK2 | Cryobacterium | WP_092324020.1 |
| | psychrotolerans | |
| PPK2 | Nocardioides | WP_091116082.1 |
| | psychrotolerans | |
| PPK2 | Pseudomonas | WP_019411115.1 |
| | psychrophile | |

In some embodiments, some or all of the CMP, UMP, GMP, NDP, and PPK kinases are heat inactivated after the reaction. In other embodiments, PPK2 enzymes used in cell-free reaction mixtures provided herein can be thermostable. For example, the PPK2 enzymes can be thermostable Class III PPK2 enzymes, which favor ATP synthesis over polyphosphate polymerization, and convert both ADP and AMP to ATP. In some embodiments, the polyphosphate kinase is a Class III PPK2 enzyme from or derived from Deinococcus geothermalis. In some embodiments, the polyphosphate kinase is the kinase represented in SEQ ID NO: 1. In some embodiments, the PPK2 enzymes are used to convert a polyphosphate, such as hexametaphosphate, to ATP, at rates ranging, for example, from 10 to 800 mM per hour (e.g., 10, 15, 20, 25, 50, 75, 100, 150, 200, 250, 300, 350, 400, 450, 500, 550, 600, 650, 700, 750, or 800 mM per hour).

The present disclosure also encompasses fusion enzymes. Fusion enzymes can exhibit multiple activities, each corresponding to the activity of a different enzyme. For example, rather than using an independent nucleoside monophosphate kinase and an independent nucleoside diphosphate kinase, a fusion enzyme (or any other enzyme) having both nucleoside monophosphate kinase activity and nucleoside diphosphate kinase activity can be used.

It should be understood that the present disclosure also embodies uses of any one or more of the enzymes described herein as well as variants of the enzymes (e.g., “PPK2 variants”). Variant enzymes can share a certain degree of sequence identity with the reference enzyme. The term “identity” refers to a relationship between the sequences of two or more polypeptides or polynucleotides, as determined by comparing the sequences. Identity measures the percent of identical matches between the smaller of two or more sequences with gap alignments (if any) addressed by a particular mathematical model or computer program (e.g., “algorithms”). Identity of related molecules can be readily calculated by known methods. “Percent (%) identity” as it applies to amino acid or nucleic acid sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Identity depends on a calculation of percent identity but can differ in value due to gaps and penalties introduced in the calculation. Variants of a particular sequence can have at least 70%, 75%, 80%, 85%, 90%, 91%, 92%, 93%, 94%, 95%, 96%, 97%, 98%, 99% but less than 100% sequence identity to that particular reference sequence, as determined by sequence alignment programs and parameters described herein and known to those skilled in the art.

The comparison of sequences and determination of percent identity between two sequences can be accomplished using a mathematical algorithm. Techniques for determining identity are codified in publicly available computer programs. Exemplary computer software to determine homology between two sequences include, but are not limited to, GCG program package (Devereu et al., 1984, Nucleic Acids Research 1: 387, 1984), the BLAST suite (Altschul et al., 1997, Nucleic Acids Res. 25: 3389), and FASTA (Altschul et al., 1990, J. Molec. Biol. 215: 403, 1990). Other techniques include: the Smith-Waterman algorithm (Smith et al., 1981, J. Mol. Biol. 147: 195; the Needleman-Wunsch algorithm (Needleman, S. B. et al., 1970, J. Mol. Biol. 48: 443; and the Fast Optimal Global Sequence Alignment Algorithm (FOGSAA) (Chakraborty et al., 2013, Sci Rep. 3: 1746, 2013).

### DNA Template

In some embodiments, the reaction comprises a DNA template encoding an mRNA to be produced according to the methods disclosed herein. A DNA template encoding an mRNA can be derived from engineered cells (e.g. on a plasmid or integrated within genomic DNA) or produced via polymerase chain reaction (PCR). In some embodiments, a DNA template is added to the cell-free reaction mixture during biosynthesis of the RNA (e.g., following a heat inactivation step). In some embodiments, DNA template concentration in a cell lysate is 0.005-1.0 g/L. In some embodiments, the DNA template concentration in a cell lysate is 0.005 g/L, 0.01 g/L, 0.1 g/L, 0.5 g/L, or 1.0 g/L.

A DNA template includes a promoter, optionally an inducible promoter. A DNA template also includes a nucleotide sequence encoding a desired RNA product (an open reading frame, or ORF) that is operably linked to the promoter. Optionally, a DNA template includes a transcriptional terminator.

A promotor or a terminator can be a naturally occurring sequence or an engineered sequence. In some embodiments, the promotor is a naturally occurring sequence. In other embodiments, the promoter is an engineered sequence. In some embodiments, the promoter is engineered to enhance transcriptional activity. In some embodiments, a terminator is a naturally occurring sequence. In other embodiments, a terminator is an engineered sequence. A DNA template can be engineered, in some instances, to have a transcriptional promoter that selectively facilitates transcription of the mRNA.

An mRNA may contain untranslated regions (UTRs) on either or both sides of the coding sequence. If positioned on the 5′ side, it is called a 5′ UTR (or leader sequence), or if positioned on the 3′ side, it is called a 3′ UTR (or trailer sequence). UTRs can have a variety of biological functions, not limited to the functions described herein. A 5′ UTR can form secondary structures that regulate translation, and in some cases can themselves be translated. A 5′ UTR advantageously comprises a sequence that is recognized by the ribosome that allows the ribosome to bind and initiate translation of the mRNA. Some 5′ UTRs have been found to interact with proteins. Some 5′ UTR sequences have been linked to mRNA localization and export signals and cellular mechanisms. Sequences and structures of 3′-untranslated regions (3′ UTRs) of messenger RNAs can govern their stability, localization, and expression. 3′ UTR regulatory elements are recognized by a wide variety of trans-acting factors that include microRNAs (miRNAs), their associated machinery, and RNA-binding proteins (RBPs). In turn, these factors instigate common mechanistic strategies to execute the regulatory programs that are encoded by 3′ UTRs.

In some embodiments the 5′UTR may include an initial transcribed sequence (ITS) positioned at the 5′ end of the 5′UTR that improves the efficiency of transcription initiation to maximize RNA product yield from transcription reactions, (e.g., cell-free reactions). An ITS is a short sequence of about 6 to 15 nucleotides. An ITS, when present, has a critical role in the early stages of transcription (initiation and the transition to elongation phase via promoter clearance) and influences the overall rate and yield of transcription from a given promoter. In some embodiments, an ITS is a naturally occurring ITS, e.g., a consensus ITS found downstream of a T7 class III promoter. In some embodiments, a consensus T7 class III promoter ITS is 6 nucleotides in length (GGGAGA). In some embodiments, an ITS is a synthetic ITS, e.g., GGGAGACCAGGAATT (SEQ ID NO: 17). In some embodiments, an ITS is 6 to 15 nucleotides of the synthetic ITS (“truncated ITS”), e.g., GGGAGACCAGGAATT (SEQ ID NO: 17).

In some embodiments, the transcribed RNA encoded by the DNA template contains one or more internal ribosomal entry site (TRES). UTRs can be strategically or empirically matched with the mRNA coding sequence to optimize translation levels and processing of mRNAs; in other words, they are modular components of the mRNA. A DNA template can contain DNA encoding for an mRNA 5? UTR sequence, an mRNA 3UTR sequence, neither, or both flanking the DNA encoding for the mRNA's open reading frame. UTR sequences used in these methods can come from multiple species and genes, and 5? and 3? sequences need not come from the same species or gene if both are present. UTR sequences can be engineered to contain specific secondary structures, binding sites, or other elements. UTRs that can be used in the methods of the invention include, but are not limited to, the UTRs listed in Table 7.

TABLE 7
Examples of UTRs

| 5′ UTR | ORF | 3′ UTR | Poly A |
|---|---|---|---|
| HSD (hydroxysteroid | Enhanced green | ALB (serum | 100 |
| 17-beta dehydro- | fluorescent protein | albumin, human) | |
| genase, human) | (EGFP) | | |
| COX (cytochrome c | Enhanced green | ALB (serum | 100 |
| oxidase subunit 6C, | fluorescent protein | albumin, human) | |
| human) | (EGFP) | | |
| HBG (hemoglobin | Enhanced green | HBG (hemoglobin | 100 |
| subunit beta, human) | fluorescent protein | subunit beta, | |
| | (EGFP) | human) | |
| XBG (beta-globin, | Enhanced green | XBG (beta-globin, | 100 |
| Xenopus laevis) | fluorescent protein | Xenopus laevis) | |
| | (EGFP) | | |
| HBG (hemoglobin | Hemagglutinin (HA) | HBG (hemoglobin | 100 |
| subunit beta, human) | from influenza A/ | subunit beta, | |
| | Puerto Rico/8/1934 | human) | |
| | (H1N1) | | |
| XBG (beta-globin, | Hemagglutinin (HA) | XBG (beta-globin, | 100 |
| Xenopus laevis) | from influenza A/ | Xenopus laevis) | |
| | Puerto Rico/8/1934 | | |
| | (H1N1) | | |
| HBG (hemoglobin | Firefly luciferase | HBG (hemoglobin | 100 |
| subunit beta, human) | (FLuc) | subunit beta, | |
| | | human) | |
| XBG (beta-globin, | Firefly luciferase | XBG (beta-globin, | 100 |
| Xenopus laevis) | (FLuc) | Xenopus laevis) | |

This disclosure contemplates DNA templates that encode a polyadenylate “tail” sequence for the mRNA at the 3′end of the resulting mRNA. In some embodiments, the polyadenylate tail is between 50 and 250 nucleotides in length. This disclosure also contemplates DNA templates for which a polyadenylate tail is not encoded. Optionally, the DNA template encodes a polyadenylation signal. In some embodiments, a polyadenylation signal is read by a polyadenylation polymerase. Optionally, the DNA template encodes a ribozyme sequence at the 3′ end of the resulting mRNA, such that the ribozyme is located 3′ to the polyadenylate tail.

In some embodiments, the DNA template is linear. A DNA template can be generated through polymerase chain reaction. A DNA template can be contained in a cassette or plasmid, including a circular plasmid, a linearized circular plasmid, or a linear plasmid. The plasmid used can be any known in the art, including but not limited to a pUC-family or pET-family, with high-copy, or medium-copy origins of replication.

The DNA template can contain a restriction endonuclease (also known as restriction enzyme) cleavage site. The restriction endonuclease for which the DNA template contains a site can be a Type IIS variety restriction endonuclease. In some embodiments, the restriction enzyme cuts in a blunt manner or results in 5′ overhang. In some embodiments, the restriction endonuclease cuts with no 3′ overhangs to avoid undesired transcriptional activity of the T7 polymerase. In some embodiments, the restriction endonuclease does not have sequence requirements to the 5′ end to the cleavage site. In some embodiments, the restriction endonuclease site is positioned so that the restriction enzyme cleaves after the polyadenylate tail-producing sequence. In some embodiments, the restriction endonuclease site is positioned so that the restriction enzyme cleaves after the polyadenylate tail-producing sequence with no additional nucleotides added after the last adenine base. In embodiments with a circular plasmid that includes a restriction endonuclease site, the circular plasmid can be treated with the corresponding restriction endonuclease to linearize it. In some embodiments, the restriction enzyme is produced by cells in culture. In some embodiments, the restriction endonuclease is prepared from a cell lysate derived from cells that produce the restriction endonuclease. In some embodiments, the plasmid DNA and restriction endonuclease are incubated together under conditions which result in linearization of the DNA template. In some embodiments, the plasmid DNA and/or restriction endonuclease are purified before or after linearization. A circular plasmid can include a transcriptional terminator sequence. A DNA template is typically provided on a vector, such as a plasmid, although other template formats can be used (e.g., linear DNA templates generated by polymerase chain reaction (PCR), chemical synthesis, or other means known in the art).

In some embodiments, more than one DNA template is used in a reaction mixture. In some embodiments, 2, 3, 4, or 5 different DNA templates are used in a reaction mixture. In some embodiments, more than one mRNA sequence is encoded in a single template.

In some embodiments, the lysate containing the DNA template is treated with a heat inactivation step before the polymerization step.

### Polymerization of Nucleoside Triphosphates to Ribonucleic Acid

After NTPs are produced as disclosed above and one or a plurality of DNA templates provided, biosynthesis of an mRNA is achieved by polymerizing NTPs into RNA (e.g., ssRNA) using, for example, a DNA-dependent RNA polymerase. In this step of the method, the DNA template is transcribed into the RNA of interest.

ATP can be produced using purified AMP or ADP plus a phosphate donor in the presence of PPK. In another aspect ATP can be produced using AMP or ADP derived from cellular RNA and a phosphate donor in the presence of PPK.

Similarly, GTP can be added directly to the reaction or purified GMP or GDP plus a phosphate donor in the presence of one or more kinases can be used to produce GTP. In yet another aspect, GTP can be produced GMP or GDP derived from cellular RNA and a phosphate donor in the presence of one or more kinases.

RNA polymerization requires NTPs, a DNA template comprising a transcriptional promoter, and a polymerase (RNA polymerase) that recognizes and commences transcription from the transcriptional promoter. Typically, a polymerase for use as provided herein is a single subunit polymerase that is highly selective for its cognate transcriptional promoters, has high fidelity, and is highly efficient. Examples of such polymerases include, without limitation, T7 RNA polymerase, T3 RNA polymerase, and SP6 RNA polymerase. Bacteriophage T7 RNA polymerase is a DNA-dependent RNA polymerase that is highly specific for the T7 phage promoters. This 99 kD enzyme catalyzes RNA synthesis from a DNA template under control of the T7 promoter. Bacteriophage T3 RNA polymerase is a DNA-dependent RNA polymerase that is highly specific for the T3 phage promoters. This 99 kD enzyme catalyzes RNA synthesis from a DNA template under control of the T3 promoter. Bacteriophage SP6 RNA polymerase is a DNA-dependent RNA polymerase that is highly specific for the SP6 phage promoter. The 98.5 kD polymerase catalyzes RNA synthesis from a DNA template under control of the SP6 promoter. Each of T7, T3, and SP6 polymerase are optimally active at 37-40° C. In some embodiments, thermostable variants of T7, T3, and SP6 polymerase are used. Thermostable variant polymerases are typically optimally active at temperatures above 40° C. (or about 40-60° C.). In some embodiments, the polymerase is not thermostable. In some embodiments, T7 polymerase is used. In some embodiments, the T7 polymerase used in the methods is purified or partially purified by precipitation and centrifugation before use in the polymerization reaction. In some embodiments, the T7 polymerase that is purified or partially purified by precipitation and centrifugation is further purified or partially purified by chromatography before use in the polymerization reaction.

TABLE 8
Examples of Polymerases

| | | GenBank # or |
|---|---|---|
| Enzyme | Organism | UniProt # |
| T7 RNA | Bacteriophage T7 | NP_041960.1 |
| Polymerase | | P00573 |
| Φ6 RdRP | Bacteriophage Φ6 | P11124 |
| T3 RNA | Bacteriophage T3 | NP_523301.1 |
| polymerase | | Q778M8 |
| SP6 Polymerase | Bacteriophage SP6 | Y00105.1 |
| | | P06221 |
| rpoA | Escherichia coli - K12 MG1655 | P0A7Z4 |
| rpoB | Escherichia coli - K12 MG1655 | P0A8V2 |
| rpoC | Escherichia coli - K12 MG1655 | P0A8T7 |

As disclosed herein, “conditions that result in production of nucleoside triphosphates and polymerization of the nucleoside triphosphates,” also referred to as “conditions for the biosynthesis of RNA” can be determined by one of ordinary skill in the art, taking into consideration, for example, optimal conditions for polymerase activity, including pH, temperature, length of time, and salt concentration of the cell lysate as well as any exogenous cofactors.

The pH of a cell-free reaction mixture during RNA biosynthesis can have a value of 3.0 to 8.0. In some embodiments, the pH value of a cell-free reaction mixture is 3-8, 4-8, 5-8, 6-8, 7-8, 3-7, 4-7, 5-7, 6-7, 3-6, 4-6, 5-6, 3-5, 3-4, or 4-5. In some embodiments, the pH value of a cell-free reaction mixture is 3.0, 3.5, 4.0, 4.5, 5.0, 5.5, 6.0, 6.5, 7.0, 7.5, or 8.0. In advantageous embodiments, the pH value of a cell-free reaction mixture during biosynthesis of RNA is 7.0 to 7.5.

The temperature of a cell-free reaction mixture during RNA biosynthesis can be 15° C. to 95° C. In some embodiments, the temperature of a cell-free reaction mixture during RNA biosynthesis is 30-60° C., 30-50° C., 40-60° C., 40-50° C., 50-70° C., 50-60° C. In some embodiments, the temperature of a cell-free reaction mixture during RNA biosynthesis is 30° C., 32° C., 37° C., 40° C., 42° C., 45° C., 50° C., 55° C., 56° C., 57° C., 58° C., 59° C., or 60° C. In advantageous embodiments, the temperature of a cell-free reaction mixture during RNA biosynthesis is 37-55° C.

A cell-free reaction mixture during RNA biosynthesis can be incubated for 15 minutes (min) to 72 hours (hrs). In some embodiments, a cell-free reaction mixture during RNA biosynthesis is incubated for 30 min-48 hrs. For example, a cell-free reaction mixture during RNA biosynthesis can be incubated for 30 min, 45 min, 1 hr, 2 hrs, 3 hrs, 4 hrs, 5 hrs, 6 hrs, 7 hrs, or 8 hrs. In some advantageous embodiments, a cell-free reaction mixture during RNA biosynthesis is incubated for 1-4 hours.

Some polymerase activities can require the presence of metal ions. Thus, in some embodiments, metal ions are added to a cell lysate. Non-limiting examples of metal ions include Mg²⁺, Li⁺, Na⁺, K⁺, Ni²⁺, Ca²⁺, Cu²⁺, and Mn²⁺. Other metal ions can be used. In some embodiments, more than one metal ion can be used. The concentration of a metal ion in a cell lysate can be 0.1 mM to 100 mM, or 10 mM to 50 mM. In some embodiments, the concentration of a metal ion in a cell lysate is 0.1, 0.2, 0.5, 1, 1.5, 2, 2.5, 3, 3.5, 4, 4.5, 5, 5.5, 6, 6.5, 7, 7.5, 8, 8.5, 9, 9.5, 10, 20, 25, 30, 35, 40, 45, 50, 60, 70, 80, 90, or 100 mM. Mg²⁺ is the preferred metal ion in the reaction.

### Thermostable Enzymes

One advantage of cell-free RNA-biosynthesis methods of the present disclosure is that all enzymes needed to convert endogenous RNA to synthetic mRNA, for example, can be (but need not be) expressed in a single engineered cell. For example, a clonal population of the engineered cell is cultured to a desired cell density, the cells are lysed, incubated under conditions that result in depolymerization of endogenous RNA to its monomer form (e.g., at a temperature of 55-99° C.), subjected to temperatures sufficient to inactivate endogenous nucleases and phosphatases (e.g., 55-99° C.), and incubated under conditions that result in the polymerization of ssRNA (e.g., 55-99° C.). In order to proceed to end product synthetic RNA, the enzymes required for conversion of NMPs to NDPs (e.g., nucleoside monophosphate kinases and/or polyphosphate kinases), from NDPs to NTPs (e.g., nucleoside diphosphate kinases and/or polyphosphate kinase), and from NTPs to RNA (e.g., polymerase) can be thermostable to avoid denaturation during heat inactivation of the endogenous nuclease (and/or exogenous nucleases) and phosphatases. Thermostability refers to the quality of enzymes to resist denaturation at relatively high temperature. For example, if an enzyme is denatured (inactivated) at a temperature of 42° C., an enzyme having similar activity (e.g., kinase activity) is considered “thermostable” if it does not denature at 42° C.

An enzyme (e.g., kinase or polymerase) is considered thermostable if the enzyme (a) retains activity after temporary exposure to high temperatures that denature other native enzymes or (b) functions at a high rate after temporary exposure to a medium to high temperature where native enzymes function at low rates.

In some embodiments, a thermostable enzyme retains greater than 50% activity following temporary exposure to relatively high temperature (e.g., higher than 41° C. for kinases obtained from E. coli, higher than 37° C. for many RNA polymerases) that would otherwise denature a similar (non-thermostable) native enzyme. In some embodiments, a thermostable enzyme retains 50-100% activity following temporary exposure to relatively high temperature that would otherwise denature a similar (non-thermostable) native enzyme. For example, a thermostable enzyme can retain 50-90%, 50-85%, 50-80%, 50-75%, 50-70%, 50-65%, 50-60%, or 50-55% activity following temporary exposure to relatively high temperature that would otherwise denature a similar (non-thermostable) native enzyme. In some embodiments, a thermostable enzyme retains 25%, 30%, 35%, 40%, 45%, 50%, 55%, 60%, 65%, 70%, 75%, 80%, 85%, 90%, 95%, 98%, 99%, or 100% activity following temporary exposure to relatively high temperature that would otherwise denature a similar (non-thermostable) native enzyme.

In some embodiments, the activity of a thermostable enzyme after temporary exposure to medium to high temperature (e.g., 42-80° C.) is greater than (e.g., 25%, 30%, 35%, 40%, 45%, 50%, 55%, 60%, 65%, 70%, 75%, 80%, 85%, 90%, 95%, 98%, 99%, or 100% greater than) the activity of a similar (non-thermostable) native enzyme.

The activity of a thermostable kinase, for example, can be measured by the amount of NMP or NDP the kinase is able to phosphorylate. Thus, in some embodiments, a thermostable kinase, at relatively high temperature (e.g., 42° C.) converts greater than 50% of NMP to NDP, or greater than 50% of NDP to NTP, in the same amount of time required to complete a similar conversion at 37° C. In some embodiments, a thermostable kinase, at relatively high temperature (e.g., 42° C.) converts greater than 60% of NMP to NDP, or greater than 60% of NDP to NTP, in the same amount of time required to complete a similar conversion at 37° C. In some embodiments, a thermostable kinase, at relatively high temperature (e.g., 42° C.) converts greater than 70% of NMP to NDP, or greater than 70% of NDP to NTP, in the same amount of time required to complete a similar conversion at 37° C. In some embodiments, a thermostable kinase, at relatively high temperature (e.g., 42° C.) converts greater than 80% of NMP to NDP, or greater than 80% of NDP to NTP, in the same amount of time required to complete a similar conversion at 37° C. In some embodiments, a thermostable kinase, at relatively high temperature (e.g., 42° C.) converts greater than 90% of NMP to NDP, or greater than 90% of NDP to NTP, in the same amount of time required to complete a similar conversion at 37° C.

The activity of a thermostable polymerase, for example, is assessed based on fidelity and polymerization kinetics (e.g., rate of polymerization). Thus, one unit of a thermostable T7 polymerase, for example, can incorporate 10 nmoles of NTP into acid insoluble material in 30 minutes at temperatures above 37° C. (e.g., at 50° C.).

Thermostable enzymes (e.g., kinases or polymerases) can remain active (able to catalyze a reaction) at a temperature of 42° C. to 99° C., or higher. In some embodiments, thermostable enzymes remain active at a temperature of 42-95° C., 42-90° C., 42-85° C., 42-80° C., 42-70° C., 42-60° C., 42-50° C., 50-80° C., 50-70° C., 50-60° C., 60-80° C., 60-70° C., or 70-80° C. For example, thermostable enzymes can remain active at a temperature of 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C. 81° C., 82° C., 83° C., 84° C., 85° C., 86° C., 87° C., 88° C. 89° C., or 90° C., 91° C., 92° C., 93° C., 94° C., 95° C., 96° C., 97° C., 98° C., or 99° C. Thermostable enzymes can remain active at relatively high temperatures for 15 minutes to 48 hours, or longer. For example, thermostable enzymes can remain active at relatively high temperatures for 1, 2, 3, 4, 5, 6, 7, 8, 9, 10, 11, 12, 13, 14, 15, 16, 17, 18, 19, 20, 24, 36, 42, or 48 hours.

Thermostable RNA polymerases can be prepared by modifying wild-type enzymes. Such modifications (e.g., mutations) are known. For example, variant thermostable T7 RNA polymerases can include one or more of the following point mutations: V426L, A702V, V795I, S430P, F849I, S633I, F880Y, C510R, and S767G (EP2377928 and EP1261696A1, each of which is incorporated herein by reference). Other variant and recombinant thermostable polymerases are encompassed by the present disclosure. Wild type T7 RNA polymerase can also be used.

In some embodiments, a thermostable T7 polymerase is used to produce an mRNA. For example, a thermostable T7 polymerase (e.g., incubated at a temperature of 40-60° C.) having a concentration of 1-2% total protein can be used to synthesize mRNA at a rate of greater than 2 g/L/hr (or, e.g., 2 g/L/hr-10 g/L/hr). As another example, a thermostable T7 polymerase (e.g., incubated at a temperature of 40-60° C.) having a concentration of 3-5% total protein can be used to synthesize mRNA at a rate of greater than 10 g/L/hr (or, e.g., 10 g/L/hr-20 g/L/hr).

It should be understood that while many embodiments of the present disclosure describe the use of thermostable enzymes, other enzymes can be used. No enzyme discussed herein need be thermostable, but thermostable variants of all enzymes discussed are included in the present disclosure. In some embodiments, purified polymerase can be exogenously added to heat-inactivated cell lysates, for example, to compensate for any reduction or loss of activity of the thermostable enzyme(s).

### Enzymatic Addition of Polyadenylate Tail

In some embodiments, a polyadenylate tail is added enzymatically using polyA polymerase, EC 2.7.7.19, also called polynucleotide adenylyltransferase. This enzyme uses RNA and ATP as substrates and catalyzes addition of A nucleotides to the 3′ end of the RNA. In some embodiments, polyadenylation with polyA polymerase is performed in a separate step after RNA synthesis, after the RNA polymerase has been inactivated, for example, through heat inactivation. In some embodiments, polyA polymerase is added at this stage, along with adenosine monophosphate (AMP), and polyphosphate. In some embodiments, the added AMP has been purified. In some embodiments, the AMP is provided as part of a mixture of NMPs or as a cell lysate.

### Engineered Cells

Engineered cells of the disclosure can comprise at least one, most, or all, of the enzymatic activities required to biosynthesize RNA. “Engineered cells” are cells that comprise at least one engineered (e.g., recombinant or synthetic) nucleic acid, or are otherwise modified such that they are structurally and/or functionally distinct from their naturally occurring counterparts. Thus, a cell that contains an engineered nucleic acid is considered an “engineered cell.”

Engineered cells of the disclosure, in some embodiments, comprise RNA, enzymes that depolymerize RNA, kinases, and/or polymerases. In some embodiments, the engineered cells further comprise a DNA template containing a promoter operably linked to a nucleotide sequence encoding an mRNA.

Engineered cells, in some embodiments, express selectable markers. Selectable markers are typically used to select engineered cells that have taken up an engineered nucleic acid following transfection of the cell (or following other procedure used to introduce foreign nucleic acid into the cell). Thus, a nucleic acid encoding product can also encode a selectable marker. Examples of selectable markers include, without limitation, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds. Additional examples of selectable markers include, without limitation, genes encoding proteins that enable the cell to grow in media deficient in an otherwise essential nutrient (auxotrophic markers). Other selectable markers can be used in accordance with the present disclosure.

An engineered cell “expresses” a product if the product, encoded by a nucleic acid (e.g., an engineered nucleic acid), is produced by the cell. It is known in the art that gene expression refers to the process by which genetic instructions in the form of a nucleic acid are used to synthesize a product, such as a protein (e.g., an enzyme).

Engineered cells can be prokaryotic cells or eukaryotic cells. In some embodiments, engineered cells are bacterial cells, yeast cells, insect cells, mammalian cells, or other types of cells. Examples include, but are not limited to, yeast, E. coli, or Vibrio cells. These cells can be sourced commercially. These cells can be grown in culture using standard high-productivity methods.

Engineered bacterial cells of the present disclosure include, without limitation, engineered Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., and Pantoea spp.

Engineered yeast cells of the present disclosure include, without limitation, engineered Saccharomyces spp., Schizosaccharomyces, Hansenula, Candida, Kluyveromyces, Yarrowia, and Pichia.

In some embodiments, engineered cells of the present disclosure are engineered Escherichia coli cells, Bacillus subtilis cells, Pseudomonas putida cells, Saccharomyces cerevisae cells, or Lactobacillus brevis cells. In some embodiments, engineered cells of the present disclosure are engineered Escherichia coli cells. As used herein, the phrase, “from” a species we mean that the gene and gene product are encoded and produced natively in that species, and that the term is intended to encompass isolation from such species and recombinant production in heterologous species, inter alia, bacteria, yeast, or other recombinant hosts.

In some embodiments, cell-free RNA-biosynthesis methods of the present disclosure can be (but need not be) expressed in a single engineered cell. For example, a clonal population of the engineered cell is cultured to a desired cell density, the cells are lysed, incubated under conditions that result in depolymerization of endogenous RNA to its monomer form (e.g., at a temperature of 30-37° C.), subjected to temperatures sufficient to inactivate endogenous nucleases and phosphatases (e.g., 40-99° C.), and incubated under conditions that result in the polymerization of ssRNA (e.g., 30-50° C.). In order to proceed to end product synthetic RNA, the enzymes required for conversion of NMPs to NDPs (e.g., nucleoside monophosphate kinases and/or polyphosphate kinases), from NDPs to NTPs (e.g., nucleoside diphosphate kinases and/or polyphosphate kinase), and from NTPs to RNA (e.g., polymerase) can be thermostable to avoid denaturation during heat inactivation of the endogenous nuclease (and/or exogenous nucleases) and phosphatases. Thermostability refers to the quality of enzymes to resist denaturation at relatively high temperature. For example, if an enzyme is denatured (inactivated) at a temperature of 42° C., an enzyme having similar activity (e.g., kinase activity) is considered “thermostable” if it does not denature at 42° C.

### Engineered Nucleic Acids

A “nucleic acid” is at least two nucleotides covalently linked together, and in some instances, can contain phosphodiester bonds (e.g., a phosphodiester “backbone”). Nucleic acids (e.g., components, or portions, of nucleic acids) can be naturally occurring or engineered. “Naturally occurring” nucleic acids are present in a cell that exists in nature in the absence of human intervention. “Engineered nucleic acids” include recombinant nucleic acids and synthetic nucleic acids. A “recombinant nucleic acid” refers to a molecule that is constructed by joining nucleic acid molecules (e.g., from the same species or from different species) and, typically, can replicate in a living cell. A “synthetic nucleic acid” refers to a molecule that is biologically synthesized, chemically synthesized, or by other means synthesized or amplified. A synthetic nucleic acid includes nucleic acids that are chemically modified or otherwise modified but can base pair with naturally occurring nucleic acid molecules. Recombinant and synthetic nucleic acids also include those molecules that result from the replication of either of the foregoing. Engineered nucleic acids can contain portions of nucleic acids that are naturally occurring, but as a whole, engineered nucleic acids do not occur naturally and require human intervention. In some embodiments, a nucleic acid encoding a product of the present disclosure is a recombinant nucleic acid or a synthetic nucleic acid. In other embodiments, a nucleic acid encoding a product is naturally occurring.

An engineered nucleic acid encoding RNA, as provided herein, can be operably linked to a “promoter,” which is a control region of a nucleic acid at which initiation and rate of transcription of the remainder of a nucleic acid are controlled. A promoter drives expression or drives transcription of the nucleic acid that it regulates.

A promoter can be one naturally associated with a gene or sequence, as can be obtained by isolating the 5′ non-coding sequences located upstream of the coding segment of a given gene or sequence. Such a promoter can be referred to as “endogenous.”

In some embodiments, a coding nucleic acid sequence can be positioned under the control of a recombinant or heterologous promoter, which refers to a promoter that is not normally associated with the encoded sequence in its natural environment. Such promoters can include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences can be produced using recombinant cloning and/or nucleic acid amplification technology, including polymerase chain reaction (PCR).

A promoter is considered to be “operably linked” when it is in a correct functional location and orientation in relation to the nucleic acid it regulates to control (“drive”) transcriptional initiation and/or expression of that nucleic acid.

Engineered nucleic acids of the present disclosure can contain a constitutive promoter or an inducible promoter. A “constitutive promoter” refers to a promoter that is constantly active in a cell. An “inducible promoter” refers to a promoter that initiates or enhances transcriptional activity when in the presence of, influenced by, or contacted by an inducer or inducing agent, or activated in the absence of a factor that causes repression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters, steroid-regulated promoters, metal-regulated promoters, pathogenesis-regulated promoters, temperature/heat-inducible, phosphate-regulated (e.g., PhoA), and light-regulated promoters.

An inducer or inducing agent can be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription can activate or inactivate transcription, depending on the regulatory system used. Activation of transcription can involve directly acting on a promoter to drive transcription or indirectly acting on a promoter by inactivation a repressor that is preventing the promoter from driving transcription. Conversely, deactivation of transcription can involve directly acting on a promoter to prevent transcription or indirectly acting on a promoter by activating a repressor that then acts on the promoter.

Engineered nucleic acids can be introduced into host cells using any means known in the art, including, without limitation, transformation, transfection (e.g., chemical (e.g., calcium phosphate, cationic polymers, or liposomes) or non-chemical (e.g., electroporation, sonoporation, impalefection, optical transfection, hydrodynamic transfection)), and transduction (e.g., viral transduction).

Enzymes or other proteins encoded by a naturally-occurring, intracellular nucleic acid can be referred to as “endogenous enzymes” or “endogenous proteins.”

### Cell Cultures and Cell Lysates

In many embodiments, engineered cells are cultured. “Culturing” refers to the process by which cells are grown under controlled conditions, typically outside of their natural environment. For example, engineered cells, such as engineered bacterial cells, can be grown as a cell suspension in liquid nutrient broth, also referred to as liquid “culture medium.”

Examples of commonly used bacterial Escherichia coli growth media include, without limitation, LB (Lysogeny Broth) Miller broth (1% NaCl): 1% peptone, 0.5% yeast extract, and 1% NaCl; LB (Lysogeny Broth) Lennox Broth (0.5% NaCl); 1% peptone, 0.5% yeast extract, and 0.5% NaCl; SOB medium (Super Optimal Broth): 2% peptone, 0.5% Yeast extract, 10 mM NaCl, 2.5 mM KCl, 10 mM MgCl₂, 10 mM MgSO₄; SOC medium (Super Optimal broth with Catabolic repressor): SOB+20 mM glucose; 2×YT broth (2× Yeast extract and Tryptone): 1.6% peptone, 1% yeast extract, and 0.5% NaCl; TB (Terrific Broth) medium: 1.2% peptone, 2.4% yeast extract, 72 mM K₂HPO₄, 17 mM KH₂PO₄4 and 0.4% glycerol; and SB (Super Broth) medium: 3.2% peptone, 2% yeast extract, and 0.5% NaCl and or Korz medium (Korz, D J et al. 1995). Examples of high-density bacterial E. coli growth media include, but are not limited to, DNAGro™ medium, ProGro™ medium, AutoX™ medium. DetoX™ medium, InduX™ medium, and SecPro™ medium.

In some embodiments, engineered cells are cultured under conditions that result in expression of enzymes or nucleic acids. Such culture conditions can depend on the particular product being expressed and the desired amount of the product.

In some embodiments, engineered cells are cultured at a temperature of 30° C. to 40° C. For example, engineered cells can be cultured at a temperature of 30° C., 31° C., 32° C., 33° C., 340 C, 35° C., 36° C., 37° C., 38° C., 39° C. or 40° C. Typically, engineered cells, such as engineered E. coli cells, are cultured at a temperature of 37° C.

In some embodiments, engineered cells are cultured for a period of time of 12 hours to 72 hours, or more. For example, engineered cells can be cultured for a period of time of 12, 18, 24, 30, 36, 42, 48, 54, 60, 66, or 72 hours. Typically, engineered cells, such as engineered bacterial cells, are cultured for a period of time of 12 to 24 hours. In some embodiments, engineered cells are cultured for 12 to 24 hours at a temperature of 37° C.

In some embodiments, engineered cells are cultured (e.g., in liquid cell culture medium) to an optical density, measured at a wavelength of 600 nm (OD₆₀₀), of 5 to 200. In some embodiments, engineered cells are cultured to an OD₆₀₀ of 5, 10, 15, 20, 25, 50, 75, 100, 150, or 200.

In some embodiments, engineered cells are cultured to a density of 1×10⁸ (OD<1) to 2×10¹¹ (OD˜200) viable cells/ml cell culture medium. In some embodiments, engineered cells are cultured to a density of 1×10⁸, 2×10⁸, 3×10⁸, 4×10⁸, 5×10⁸, 6×10⁸, 7×10⁸, 8×10⁸, 9×10¹⁰, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, or 2×10¹¹ viable cells/ml. (Conversion factor: OD 1=8×10⁸ cells/ml).

In some embodiments, engineered cells are cultured in a bioreactor. A bioreactor refers simply to a container in which cells are cultured, such as a culture flask, a dish, or a bag that can be single-use (disposable), autoclavable, or sterilizable. The bioreactor can be made of glass, or it can be polymer-based, or it can be made of other materials. Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor can be a batch or continuous processes and will depend on the engineered cells being cultured. A bioreactor is continuous when the feed and product streams are continuously being fed and withdrawn from the system. A batch bioreactor can have a continuous recirculating flow, but no continuous feeding of nutrient or product harvest. For intermittent-harvest and fed-batch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product can be harvested, and the removed culture medium is replenished with fresh medium. This process can be repeated several times. For production of recombinant proteins and antibodies, a fed-batch process can be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the production phase. Fresh medium can be added proportionally to cell concentration without removal of culture medium (broth). To accommodate the addition of medium, a fed-batch culture is started in a volume much lower that the full capacity of the bioreactor (e.g., approximately 40% to 50% of the maximum volume).

Some methods of the present disclosure are directed to large-scale production of RNA (e.g., ssRNA, more specifically mRNA). For large-scale production methods, engineered cells can be grown in liquid culture medium in a volume of 5 liters (L) to 250,000 L, or more. In some embodiments, engineered cells can be grown in liquid culture medium in a volume of greater than (or equal to) 10 L, 100 L, 1000 L, 10000 L, or 100000 L. In some embodiments, engineered cells are grown in liquid culture medium in a volume of 5 L, 10 L, 15 L, 20 L, 25 L, 30 L, 35 L, 40 L, 45 L, 50 L, 100 L, 500 L, 1000 L, 5000 L, 10000 L, 100000 L, 150000 L, 200000 L, 250000 L, or more. In some embodiments, engineered cells can be grown in liquid culture medium in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L. In some embodiments, engineered cells can be grown in liquid culture medium in a volume of 100 L to 300000 L, 100 L to 200000 L, or 100 L to 100000 L.

Typically, engineered cell culturing is followed by lysing the cells. “Lysing” refers to the process by which cells are broken down, for example, by viral, enzymatic, mechanical, or osmotic mechanisms. A “cell lysate” refers to a fluid containing the contents of lysed cells (e.g., lysed engineered cells), including, for example, organelles, membrane lipids, proteins, nucleic acids and inverted membrane vesicles. Cell lysates of the present disclosure can be produced by lysing any population of engineered cells, as provided herein.

Methods of cell lysis, referred to as “lysing,” are known in the art, any of which can be used in accordance with the present disclosure. Such cell lysis methods include, without limitation, physical lysis such as homogenization.

Cell lysis can disturb carefully controlled cellular environments, resulting in protein degradation and modification by unregulated endogenous proteases and phosphatases. Thus, in some embodiments, protease inhibitors and/or phosphatase inhibitors can be added to the cell lysate or cells before lysis, or these activities can be removed by heat inactivation or gene inactivation.

Cell lysates, in some embodiments, can be combined with at least one nutrient. For example, cell lysates can be combined with Na₂HPO₄, KH₂PO₄, NH₄Cl, NaCl, MgSO₄, or CaCl₂). Examples of other nutrients include, without limitation, magnesium sulfate, magnesium chloride, magnesium orotate, magnesium citrate, potassium phosphate monobasic, potassium phosphate dibasic, potassium phosphate tribasic, sodium phosphate monobasic, sodium phosphate dibasic, sodium phosphate tribasic, ammonium phosphate monobasic, ammonium phosphate dibasic, ammonium sulfate, ammonium chloride, and ammonium hydroxide.

Cell lysates, in some embodiments, can be combined with at least one cofactor. For example, cell lysates can be combined with adenosine diphosphate (ADP), adenosine triphosphate (ATP), nicotinamide adenine dinucleotide (NAD+), or other non-protein chemical compounds required for activity of an enzyme (e.g., inorganic ions and coenzymes).

In some embodiments, cell lysates are incubated under conditions that result in RNA depolymerization. In some embodiments, cell lysates are incubated under conditions that result in production of ssRNA or more particularly mRNA.

The volume of cell lysate used for a single reaction can vary. In some embodiments, the volume of a cell lysate is 0.001 to 10 m³. For example, the volume of a cell lysate can be 0.001 m³, 0.01 m³, 0.1 m³, 1 m³, 5 m³, 10 m³

In some embodiments, cell lysates are further processed before RNA depolymerization. Total cellular RNA can be recovered from cultured cells using established techniques, for instance, use of TRIzol reagent or salt, or precipitation.

### Capping

An mRNA cap serves a variety of functions, including, but not limited to, recruiting ribosomal subunits, promoting ribosome assembly & translation, and protecting the mRNA from exonuclease activity.

Capping can be achieved using a variety of methods. In some embodiments, capping is achieved using one or more enzymes. The process of capping requires a variety of enzymatic activities that are represented in Table 9. In some embodiments, one protein accomplishes all four functions. In some embodiments, the four activities are accomplished by two, three, or four enzymes.

TABLE 9
Capping enzyme activity

| Activity | Enzyme name | EC # |
|---|---|---|
| A | RNA 5′-triphosphatase | 3.1.3.33 |
| B | Guanylyltransferase | 2.7.7.50 |
| C | Guanylyl methyltransferase | 2.1.1.56 |
| D | 2′-O-Methyltransferase | 2.1.1.57 |

Capping can be performed after the RNA polymerization step. In some embodiments, the RNA polymerization reaction is deactivated before capping. In some embodiments, the capping enzymes are added to the reaction, along with a methyl donor (e.g., S-adenosylmethionine), and either GTP or GMP with polyphosphate. In some embodiments, GMP is converted to GTP by kinases present in the reaction. Messenger RNA can be capped using a variety of enzymes. A non-comprehensive list of enzymes for potential use in capping messenger RNAs that can be used in the methods of the invention is included in Table 10.

TABLE 10
Examples of Capping Enzymes

| | Protein Name | Organism | Virus Class |
|---|---|---|---|
| 1 | D1 | Vaccinia virus | Pox |
| 2 | D12 | Vaccinia virus | Pox |
| 3 | VP39 | Vaccinia virus | Pox |
| 4 | VP4-1 | Bluetongue virus 10 | Orbivirus |
| 5 | VP4-2 | Cronobacter | N/A |
| | | malonaticus | (Entero- |
| | | | bacteriaceae) |
| 6 | MIMI_R382 | Acanthamoeba | Mimiviridae |
| | | polyphaga mimivirus | |
| 7 | Ba71V-101 | African swine fever | Asfivirus |
| | | virus Ba71V | |
| 8 | PLM | Powai lake megavirus | Mimiviridae |
| 9 | Cap | Wallal virus | Orbivirus |
| | | | (Reovirus) |
| 10 | VP4-3 | African horse sickness | Orbivirus |
| | | virus 4 | (Reovirus) |
| 11 | VP4-4 | Epizootic | Orbivirus |
| | | hemorrhagic disease | (Reovirus) |
| | | virus | |
| 12 | VP4-5 | Grass carp reovirus | Reovirus |
| 13 | NS5 | West Nile virus | Flavivirus |
| 14 | NS3-T | West Nile virus | Flavivirus |
| 15 | MTase | Acanthamoeba | Mimiviridae |
| | | polyphaga mimivirus | |
| 16 | Ba71V-055 | African swine fever | Asfivirus |
| | | virus Ba71V | |
| 17 | VP3-BTV | Bluetongue virus 10 | Orbivirus |
| 18 | VP7-BTV | Bluetongue virus 10 | Orbivirus |
| 19 | D1- | Vaccinia (fusion) | Pox |
| | GSSGGSGSGGSSSGS- | | |
| | D12 | | |

In some embodiments, mRNA is capped using a cap analog. Cap analogs can include dinucleotide cap analogs (e.g. standard cap analog or anti-reverse cap analog, ARCA) or 3+ nucleotide cap analogs (e.g. CleanCap from TriLink), unmethylated cap analogs, or methylated cap analogs. In some embodiments, the cap analog is added to the polymerization reaction.

In some embodiments, one or more internal ribosomal entry site (IRES) sequences is included instead of or in addition to a cap. In some embodiments, the IRES sequence is incorporated into the 5′ UTR sequence. In some embodiments, an IRES is from a viral genome, such as Encephalomyocarditis virus (EMCV) or Cricket Paralysis Virus (CrPV). In some embodiments, an IRES is from a cellular mRNA, such as those encoding apoptotic protease activating factor (Apaf-1), myelin transcription factor 2 (MYT-2), or c-myc.

Capping can be performed at a variety of different steps of the mRNA synthesis process. Capping can occur co-transcriptionally or post-transcriptionally. These methods can be executed after RNA synthesis and before or after an enzymatic polyadenylation step, if enzymatic polyadenylation is performed. In some embodiments, the RNA is capped in the reaction mix, before purification. In other embodiments, the RNA is capped after it is purified.

In some embodiments, auxiliary enzymes, such as 5′, 3′ exoribonuclease 1 (xrn1), can be used to achieve more efficient capping. Xrn1 treatment allows for the degrading of mRNA starting with a GMP, which cannot be capped, back into NMP monomers, which can then be used again to produce mRNA. Since this degradation does not affect mRNA starting with a GTP or GDP, this will increase the production of mRNA that can be capped. In some embodiments, monophosphorylated mRNA recycling using specific exonuclease is performed. In addition to the standard enzymes in the CFR reaction, 5′-monophosphate specific exoribonucleases such as xrn1 are added. Xrn1 can be used to degrade mRNA that has a 5′ monophosphate, recycling the NMPs back into the CFR reaction. It can also be used to degrade uncappable mRNA in mRNA produced in a CFR.

### Downstream Processing

The methods and systems provided herein, in some embodiments, yield mRNA product at a concentration of 0.5-10 g/L (e.g., 0.5, 1, 2, 3, 4, 5, or 10 g/L). Downstream processing increases purity to as much as 99% (e.g., 75, 80, 85, 90, 95, 96, 97, 98, or 99%) RNA by weight. An example of downstream processing is shown in starting with the addition of a protein precipitating agent (e.g., lithium chloride) followed by disc-stack centrifugation (DSC) to remove protein, lipids, and some DNA from the product stream. Ultrafiltration is then implemented to remove salts and volume. Addition of lithium chloride to the product stream leads to precipitation of the RNA product, which is subsequently separated from the bulk liquid using disc stack centrifugation, for example, yielding an ˜80% purity RNA product stream. Further chromatographic polishing yield about 99% pure product.

In some embodiments, the mRNA product is precipitated by lithium chloride precipitation protocols. In some embodiments, the mRNA product is ultra purified through the reversed-phase ion-pair high performance liquid chromatography protocol described in Weissman et al., 2013, “HPLC purification of in vitro transcribed long RNA.” Methods in molecular biology (Clifton, N.J.), 969, 43. Reversed-phase HPLC can be used, in some embodiments, to remove contaminating nucleic acid products, for example, double-stranded nucleic acids which can lead to undesired immune responses, from the mRNA preparation. Other purification methods can also be used.

### EXAMPLES

The Examples that follow are illustrative of specific embodiments of the disclosure, and various uses thereof. They are set forth for explanatory purposes only and should not be construed as limiting the scope of the disclosure in any way.

### Example 1. Cell-Free Reactions

Cell-free RNA synthesis reactions were assembled from the following components:

NMP mix: yeast RNA treated with Nuclease P1 to yield 5′ nucleoside monophosphates (NMPs). Nuclease P1 was removed by ultrafiltration and the mixture adjusted to neutral pH prior to use in the cell-free reactions.

Kinases: E. coli strains overexpressing the following kinases were grown in high-cell density fermentations and lysed by high-pressure homogenization:
CMP kinase (Cmk) from Thermus thermophilus
UMP kinase (PyrH) from Pyrococcus furiosus
GMP kinase (Gmk) from Thermotoga maritima
NDP kinase (Ndk) from Aquifex aeolicus
Polyphosphate kinase (PPK2) from Deinococcus geothermalis
   RNA polymerase: Thermostable mutant T7 RNA polymerase with an N-terminal hexahistidine tag was overexpressed in E. coli in high-cell density fermentation. (Wild type T7 RNA polymerase was also successfully used in these reactions.) Cells were lysed by high-pressure homogenization and the protein purified by Fast Protein Liquid Chromatography (FPLC).

Cell-free RNA synthesis reactions were assembled according to Table 11. Prior to reaction assembly, kinase lysates were diluted to equal total protein concentrations in potassium phosphate buffer and combined. Magnesium sulfate and sodium hexametaphosphate were added, and then the lysate mix heat-treated (70° C. for 15 minutes) to inactivate off-pathway enzymatic activities from the kinase lysates.

TABLE 11
Example Reaction Setup

| Component | Concentration |
|---|---|
| NMP mix | 20% v/v |
| MgSO₄ | 45 mM |
| Sodium hexametaphosphate | 13 mM |
| (HMP) | |
| RNA polymerase | 0.1 mg/mL |
| Template DNA | 20 ng/μL |
| Kinase lysate mix | 1.75 mg/mL total |
| | protein |

CFRs were incubated at 48° C. for 60 minutes, then treated with TURBO DNase (Thermo Fisher). Insoluble debris was pelleted by centrifugation, and the supernatant transferred to a new tube. RNA was recovered by lithium chloride precipitation following standard techniques.

### Example 2: Capping of mRNA

The cell lysate produced in Example 1 was subjected to a capping reaction using a commercial kit (e.g. Vaccinia capping system, New England Biolabs) containing Vaccinia virus enzymes. The reaction was performed as recommended by the product specification.

### Example 3: PCR Production of Linear Template

DNA template sequences containing the open reading frames (ORF) and untranslated regions (UTRs) were synthesized as linear dsDNA gBlocks (Integrated DNA Technologies) and cloned into pCR4-TOPO (Thermo Fisher Scientific). Linear DNA templates were amplified by PCR using a reverse primer that encoded the polyA tail. PCR products were purified using AMPure XP SPRI beads (Beckman Coulter). This template DNA is used in the procedure described in Example 1.

### Example 4: GFP Expression in HeLa Cells

HeLa cells were transfected with 0.1 μg mRNA encoding green fluorescent protein (GFP) with the XBG (beta-globin, Xenopus laevis) UTR, and a 5′ ITS, produced either through the cell-free process described in the previous Examples, crudely purified with lithium chloride precipitation, or by in vitro transcription (IVT). 3% MessengerMAX lipofectamine, as per manufacturer instructions, was used for transfection. Green fluorescent protein expression was compared.

### Results

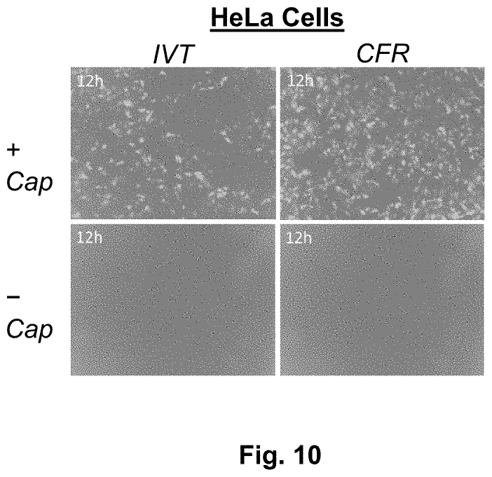

CFR resulted in green fluorescent protein expression equivalent to that produced by IVT (FIG. 10). In summary the results demonstrate that the cell-free reaction method is comparable to the in vitro transcription (IVT) method.

### Example 5: Expression with Different UTRs

mRNA encoding GFP was produced using by CFR and assayed in HeLa cell extracts. mRNAs with 4 different untranslated regions (UTRs)—5′ hydroxysterol dehydrogenase, 3′ albumin (HSD); 5′ cytochrome oxidase, 3′ albumin (COX); 5′, 5′ human β-globin (HBG); 5′, 3′ Xenopus β-globin (XBG), each with 5′ITS—were prepared. (These UTRs are detailed in Table 7.) Expression of GFP was quantified by monitoring green fluorescence of the reaction (e.g. in a qPCR machine or fluorescence microplate reader with the appropriate settings).

### Results

mRNAs with all four UTRs produced by CFR resulted in active GFP expression (FIG. 11). XBG, HBG, COX, and HSD UTRs all resulted in expression at or above 300,000 RFUs of GFP.

### Example 6: RNA Purification

mRNA from the CFR process and mRNA that was produced through in vitro transcription (IVT) was crudely purified using lithium chloride precipitation. The relative abundance of the RNAs was determined using capillary gel electrophoresis on a BioAnalyzer. mRNA made by CFR and either only crudely purified through lithium chloride precipitation or purified with reversed-phase ion-pair high performance liquid chromatography (ultra-purification or “polishing”) followed by lithium chloride precipitation as follows.

HPLC purification essentially followed the protocol of Weissman et al., 2013, “HPLC purification of in vitro transcribed long RNA.” Methods in molecular biology (Clifton, N.J.), 969, 43.

An RNASep Semi-Prep (ADS Biotec Catalog #RPC-99-2110) 21.2×100 mm was used, maintained at 80° C. The mobile phases used were as follows:

A—0.1 M tetraethylammonium acetate (TEAA), pH 7.0

B—0.1M TEAA, pH 7.0, 25% acetonitrile

Samples were injected onto the column and separated using the following gradient program with a pump speed of 3 ml/min:

0.0 min, 62% A, 38% B

20.0 min, 40% A, 60% B

20.01 min, 0% A, 100% B

22.00 min, 0% A, 100% B

22.01 min, 62% A, 38% B

28.0 min, 62% A, 38% B.

Fractions containing the desired mRNA species were pooled and concentrated using a pre-wetted 30 kDa cutoff centrifugal filter (e.g. Amicon UFC903096). The concentrated mRNA sample was precipitated with LiCl following standard techniques.

The crudely purified or ultra-purified CFR mRNA was tested for dsRNA using an immunoblotting technique according to Karikó et al., 2011, “Generating the optimal mRNA for therapy: HPLC purification eliminates immune activation and improves translation of nucleoside-modified, protein-encoding mRNA.” Nucleic Acids Research, 39(21), e142. The crude and ultra-purified CFR mRNA was also tested for endotoxin using the EndoSafe-MCS system (Charles River Laboratories, manufacturer's instructions) and compared to crude or ultra-purified mRNA produced through IVT. The products of crude and ultra-purification were subjected to mass-based purity analysis as below.

Residual protein was quantified by bicinchoninic acid (BCA) assay kit (Thermo Fisher), following kit instructions. Residual cations were quantified by the method of Thomas et al., 2002, “Determination of inorganic cations and ammonium in environmental waters by ion chromatography with a high-capacity cation-exchange column.” Journal of Chromatography A, 956(1-2), 181-186. Residual anions were quantified by the method of Boyles, 1992, “Method for the analysis of inorganic and organic acid anions in all phases of beer production using gradient ion chromatography.” Journal of the American Society of Brewing Chemists, 50(2), 61-63. Residual nucleotides were quantified following the methods of Edelson et al., 1979, “Ion-exchange separation of nucleic acid constituents by high-performance liquid chromatography.” Journal of Chromatography A, 174(2), 409-419; and Hartwick et al., 1975, “The performance of microparticle chemically-bonded anion-exchange resins in the analysis of nucleotides.” Journal of Chromatography A, 112, 651-662.

### Results

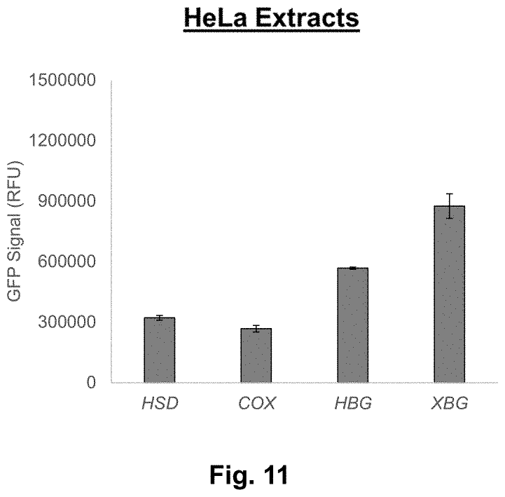

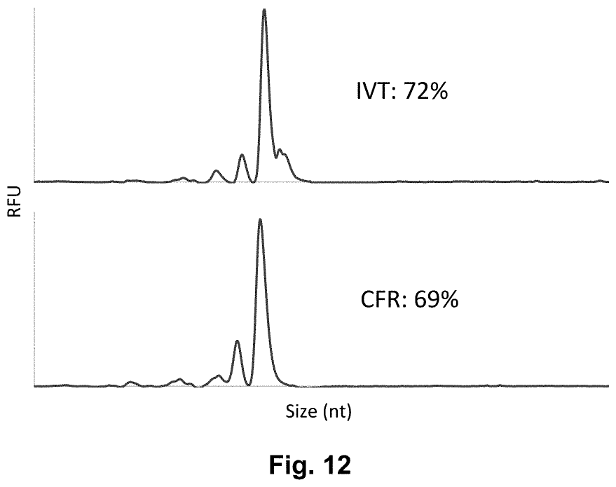

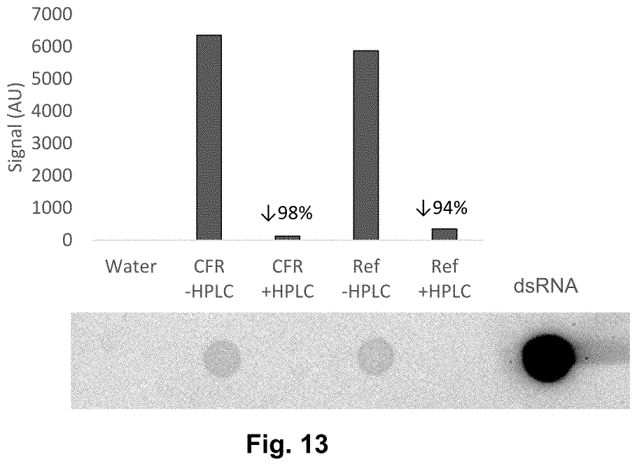

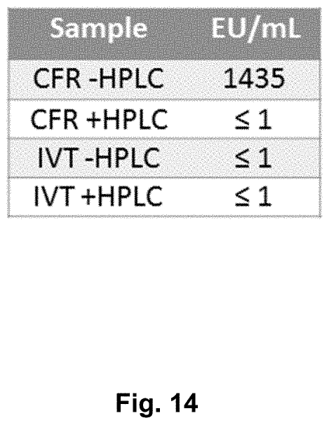

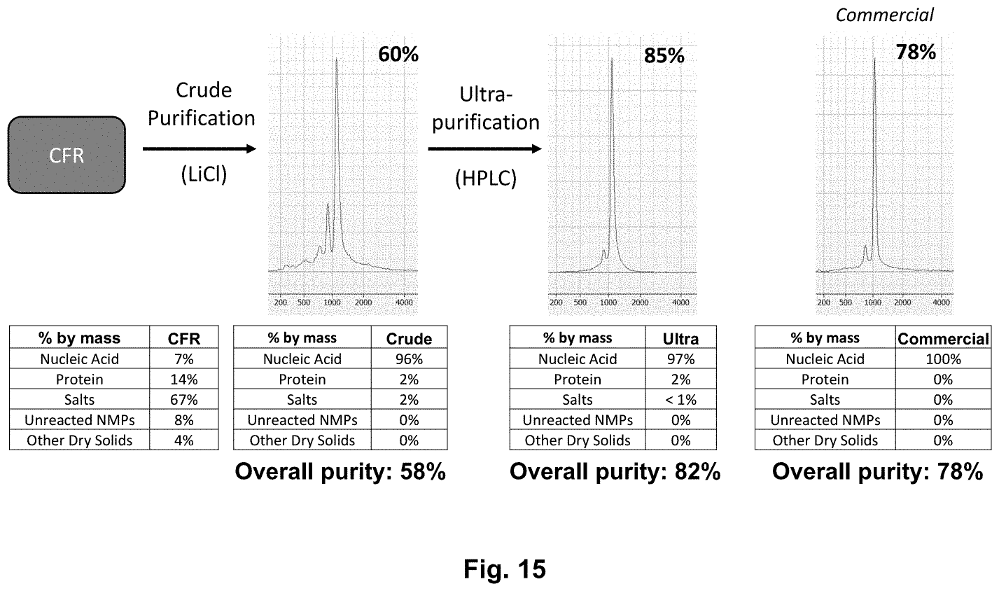

mRNA made through the cell-free process results in nucleic acid purity of less than 70% of the desired mRNA, though the purity achieved is similar to that achieved with in vitro transcription (IVT; FIG. 12). The HPLC process removes most of the dsRNA (FIG. 13). Endotoxin is removed (FIG. 14). Subsequent “polishing” of the RNA using the HPLC process results in a substantially higher percentage of total nucleic acid representing the mRNA species of interest—85%. The commercial RNA preparation contained the species of interest at 78% (FIG. 15).

### Example 7: Expression of Influenza Antigen

mRNAs encoding the hemagglutinin protein (HA) from H1N1 Puerto Rico/8/1934 influenza virus, with either HBG or XBG UTRs, each including a 5′ ITS, were produced using the CFR system and either LiCl-precipitated or ultra-purified using HPLC. HA was measured in HeLa extracts using ELISA. Microplates were first coated with capture antibody (Rabbit anti-Influenza A/Puerto Rico/8/1934 hemagglutinin monoclonal antibody, Sino Biological), then washed and blocked. HeLa cell extracts containing translated HA were diluted, then applied to the plate and incubated for 1 hour at room temperature. Wells were then washed before detection antibody (Rabbit anti-Influenza A/Puerto Rico/8/1934 conjugated to horseradish peroxidase, Sino Biological) was applied. Horseradish peroxidase substrate (3′, 3′, 5′, 5′-tetramethylbenzidine) was then added, and absorbance at 650 nm quantified using a microplate reader.

The CFR-produced mRNA was analyzed by Western Blot in comparison to mRNA produced by in vitro transcription (IVT). Translated HA was also detected in cell extracts by Western blotting, using an affinity-purified rabbit anti-Influenza A/Puerto Rico/8/1934 polyclonal primary antibody (Sino Biological) and an affinity-purified goat anti-rabbit horseradish peroxidase conjugated secondary antibody (Jackson Immunologicals).

### Results

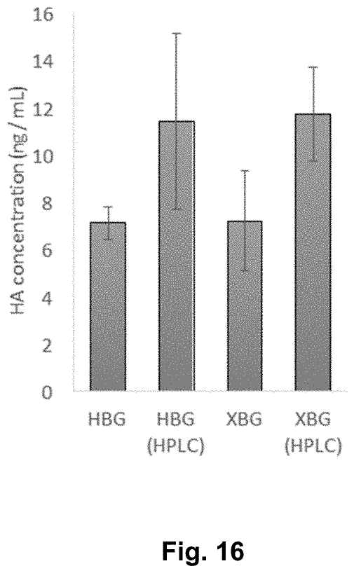

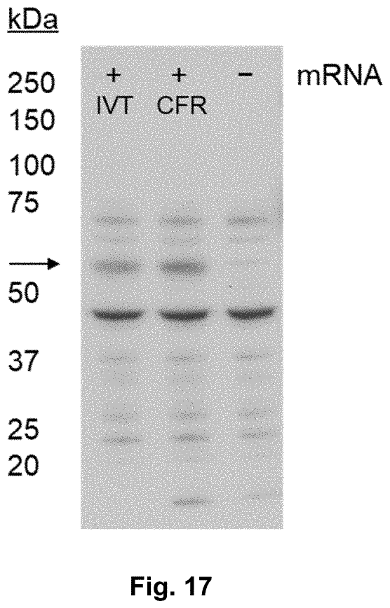

HA was successfully produced, and the concentration in the final preparation was higher if the sample was subjected to ultra-purification (FIG. 16). Production was similar to that achieved with IVT. (FIG. 17).

### Example 8: Cell-Free Production of Luciferase

mRNAs encoding firefly luciferase with either HBG or XBG UTRs, each including a 5′ ITS, were produced using the CFR process. Translation was measured in HeLa cell extracts using the 1-Step Human Coupled IVT Kit (Thermo Fisher) following kit instructions, except that 500 ng of capped, DNase-treated mRNA (corrected for purity) was added instead of purified DNA. Expression of firefly luciferase was measured using the Steady-Glo Luciferase Assay System (Promega), following kit instructions. Luminescence from the firefly luciferase was measured in both HeLa extracts and HeLa cells, with the Promega kit providing readout. The HeLa cell transfection was performed as in Example 4.

### Results

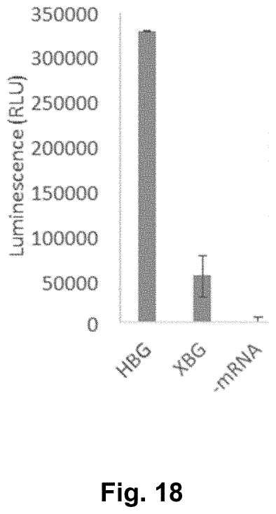

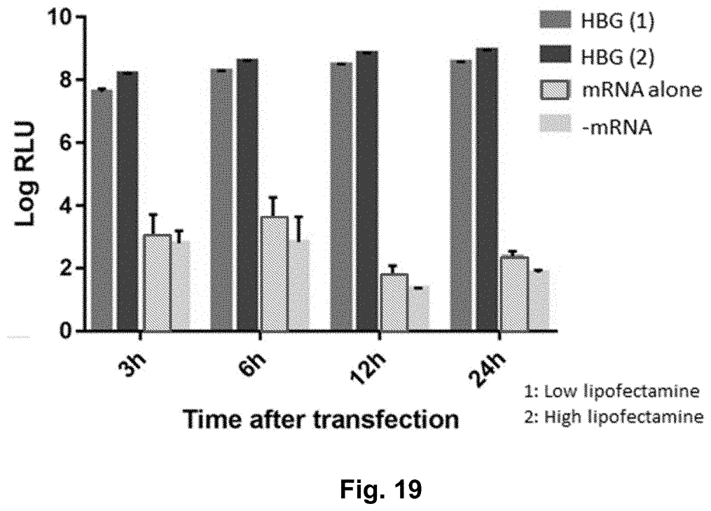

Functional luciferase was produced in both HeLa extracts (FIG. 18) and HeLa cells (FIG. 19). In HeLa extracts, higher luciferase expression was observed with HBG UTRs compared to XBG. In HeLa cells, luciferase mRNA with HBG UTRs produced high levels of luciferase expression regardless of transfection condition (1: 0.15 uL lipofectamine per well, 2: 0.30 uL lipofectamine per well, with 100 ng mRNA transfected in each case)

### Example 9: Expression of Cell-Free RNA-Produced Luciferase In Vivo

mRNA encoding firefly luciferase was produced using both IVT and CFR methods, HPLC-purified, and capped. mRNAs included HBG UTRs and a 5′ ITS. mRNA was produced in two formulations for both CFR and IVT: “in-house” lipid nanoparticles (literature formulation, optimized by the researchers) and external lipid nanoparticles (made by a commercial partner or with the Precision Nanosystems kit). “In-house” LNPs were formulated according to Pardi et al. using D-Lin-MC3-DMA as the ionizable lipid: Pardi et al., 2015, J. Controlled Release, 217, 345-351. “GenVoy” LNPs were formulated using the GenVoy-ILM Ionizable Lipid Mix (Precision Nanosystems). Both formulations were produced using the NanoAssemblr Benchtop microfluidic mixer (Precision Nanosystems) following manufacturer instructions.

Each of the 4 treatments (2 production methods, 2 formulations) was administered to an experimental group of 3 BALB/c mice in a single intradermal dose of 40, 15, or 5 μg. Animals were administered D-luciferin 150 mg/kg intraperitoneally and imaged with an in vivo imaging system (IVIS) 6 hours after mRNA administration, and luminescence was measured over 72 hours.

### Results

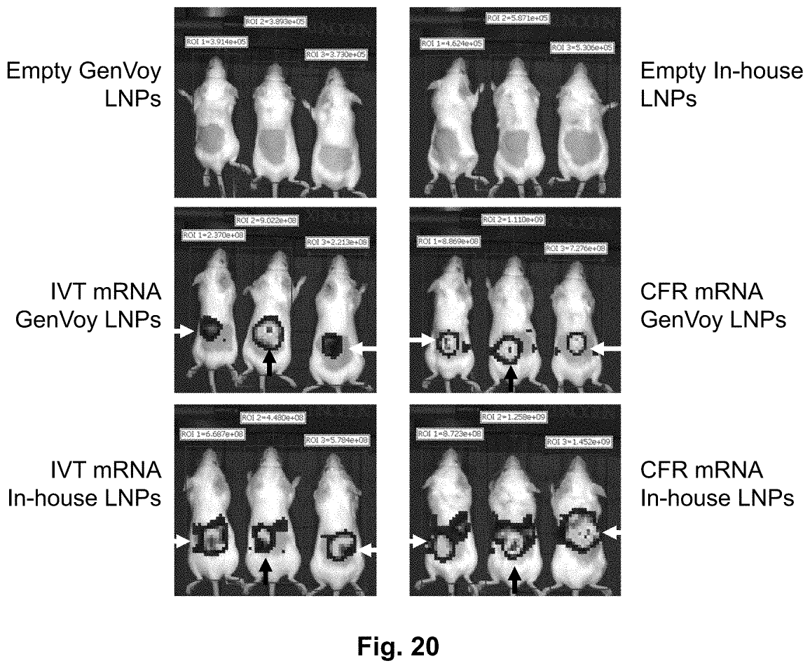

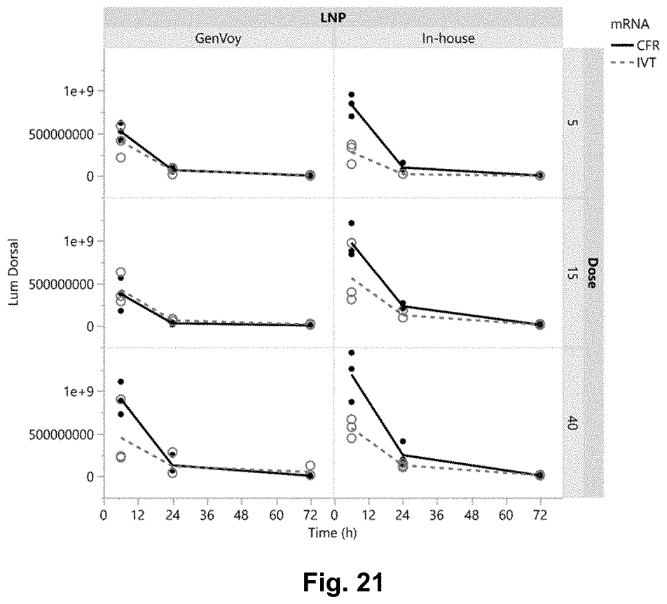

CFR-produced mRNA is at least as potent as IVT in eliciting luciferase expression. At earlier time points, the CFR-produced mRNA with the internal formulation yielded higher luciferase expression. Similar levels of expression are achieved with the 40 and 15 μg administrations (FIG. 20, 21).

### Example 10: Production of Nucleoside-Modified mRNAs with Anti-Reverse Cap Analog (ARCA) Capping

Nucleoside-modified mRNAs were produced using CFR & HPLC purified as described in Example 9, with the exception that the nucleotide source for the synthesis reaction consisted of the unmodified nucleoside monophosphates adenosine and guanosine and pseudouridine, y and 5-methylcytidine, ^{m5}C. Unmodified and modified nucleosides were added to the reaction at 5 mM each, with the exception of GMP, which was added at 1 mM. Cap analog (ARCA) was added at 4 mM. Reactions were incubated for 4 hours at 37° C., after which reactions were DNase-treated, recovered by LiCl precipitation and purified by HPLC. Templates were produced by PCR as described previously in the application and contained the gene of interest (firefly luciferase) flanked by 5′ and 3′ untranslated regions, as well as a 3′ polyA tail. mRNAs were subsequently analyzed for purity, incorporation of nucleoside modifications, capping, and gene expression in mice.

Quantification of nucleoside modification was achieved by digesting samples to mononucleosides using a mixture of nuclease, phosphodiesterase, and phosphatase enzymes, and mononucleosides quantified using LC-MS. Relative concentrations of modified nucleoside (e.g. ψ) were compared to unmodified (e.g. U). Similarly, for the ^{m7}G cap, relative concentrations of ^{m7}G were compared to the IVT reference.

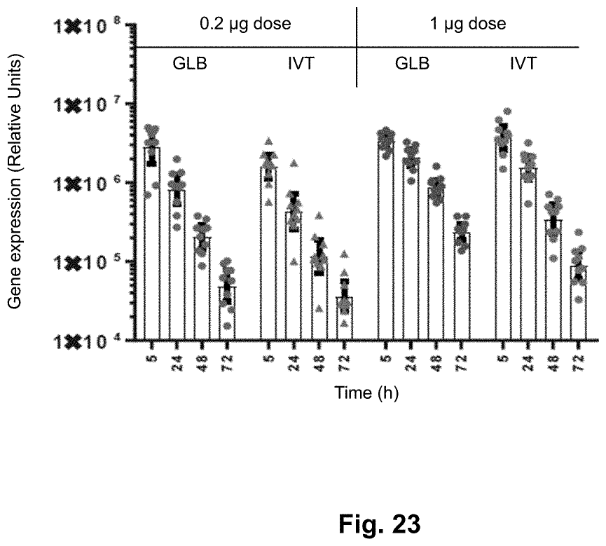

Determination of target gene expression in mice was achieved by formulating mRNAs into lipid nanoparticle formulations followed by injection at the doses indicated into BALB/c mice (N=10 mice per group) via the intramuscular route. Mice were subsequently injected with D-luciferin at times indicated in FIG. 23, followed by in vivo imaging (IVIS).

### Results

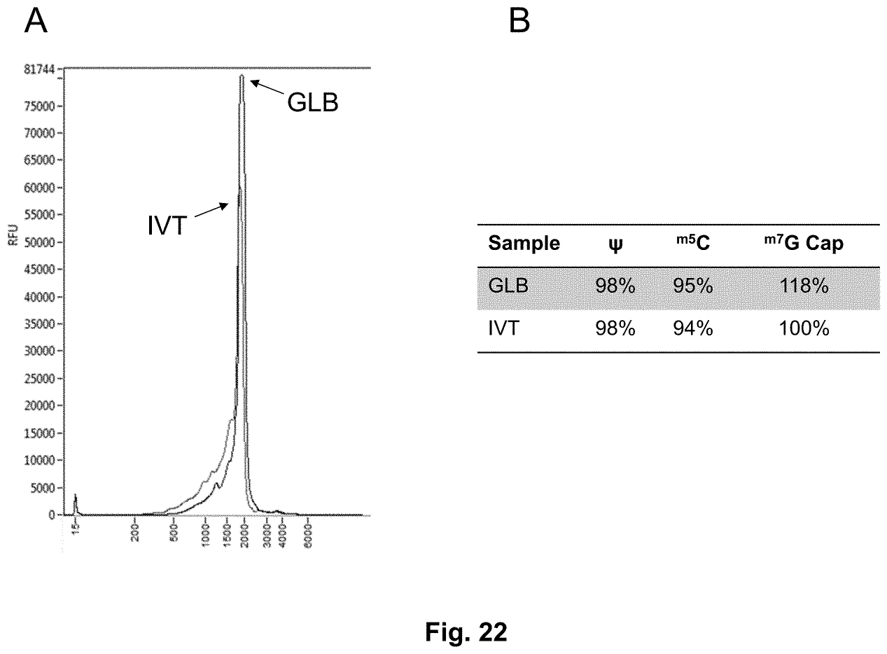

Similar purity was observed in CFR-produced mRNA and a reference standard of the same sequence produced by IVT (FIG. 22A). Quantification of nucleoside modification and capping of CFR-produced mRNA and the reference standard of the same sequence produced by IVT are shown in FIG. 22B. CFR-produced mRNA exhibited similar substitution efficiency with modified nucleosides and similar capping to IVT. FIG. 23 provides a graphical depiction of target gene expression in mice. At both 1 μg and 0.1 μg doses, CFR-produced mRNAs were similarly potent to IVT at all timepoints.

### Example 11: Production of a Model Influenza Vaccine that Protects Mice from Influenza Infection

mRNAs encoding a model influenza vaccine were produced using CFR, HPLC-purified, and encapsulated in lipid nanoparticles as described in Example 9 of this application. mRNAs encoded the full-length hemagglutinin (HA) protein from influenza A/Puerto Rico/8/1934(H1N1) according to Petsch et al., Nat Biotechnol. 2012 (doi:10.1038/nbt.2436), or firefly luciferase (FLuc). mRNA sequences included 5′ and 3′ HBG UTRs, a 5′ ITS, and a 3′ A₁₀₀ tail. Templates were produced by PCR. BALB/c mice (N=8 mice per group) were immunized twice at the indicated doses (prime immunization at Day 0 and boost at Day 21; intramuscular). Mice administered inactivated H1N1 virus served as positive controls. Serum immunity was quantified in treated mice, and protection from influenza challenge measured. Serum immunity was determined in mice by hemagglutination inhibition (HAI) assay. Blood was collected by tail vein at Day 42 and processed to serum for HAI determination. Influenza challenges was measured by body weight changes in mice after challenge with influenza A/Puerto Rico/8/1934 (H1N1). Mice were administered live virus intranasally on Day 63 and body weights monitored for 10 days thereafter.

### Results

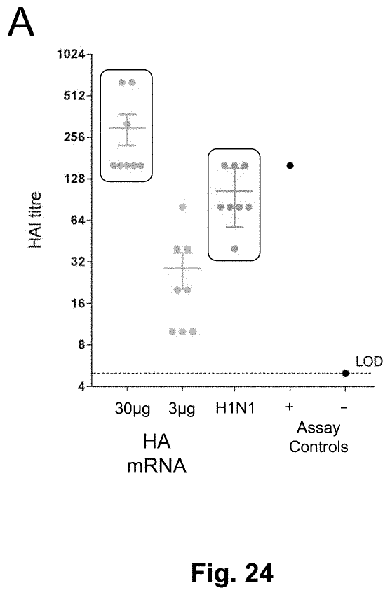

Serum immunity in mice, as measured by the HAI assay, is shown in FIG. 24. Mice treated with both doses of HA mRNA produced HA-inactivating antibodies, with titers in the 30 μg dose group exceeding those of the inactivated H1N1 control group. Mice from these groups (circled in FIG. 24) were selected for the subsequent challenge study.

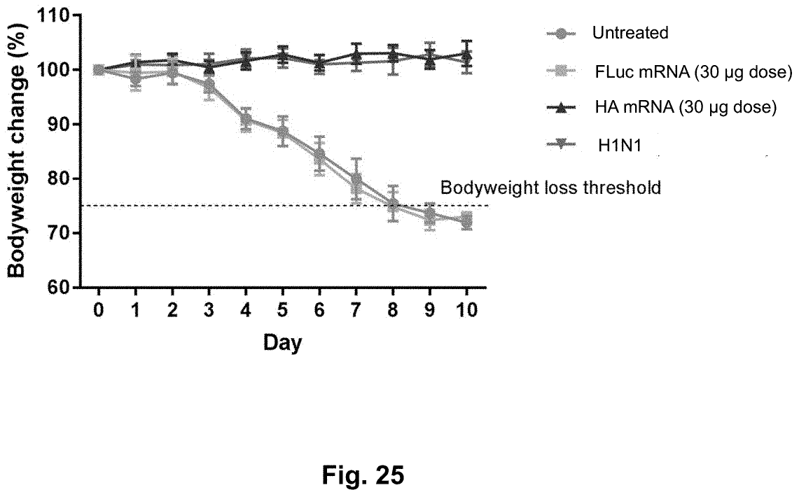

Body weights of mice after challenge with influenza A/Puerto Rico/8/1934 (H1N1) are shown in FIG. 25. Mice administered HA mRNA or the inactivated H1N1 control were protected from body weight loss associated with influenza infection, while untreated mice and mice administered FLuc mRNA lost weight until they reached the humane study endpoint (25% total body weight loss) and were sacrificed.

These results demonstrate that CFR-produced mRNA produced an effective immune response against influenza A/Puerto Rico/8/1934, and can therefore act as a protective and specific vaccine in mice.

### Example 12: Production of mRNA from Various Nucleotide Sources

mRNAs encoding influenza hemagglutinin, described in Example 11, were produced by CFR utilizing cellular RNA-derived nucleotides or using purified nucleoside monophosphates. mRNA was produced as described in Example 9 of this application, except that cellular RNA-derived nucleotide mixture was pre-incubated with the kinases, magnesium, and sodium hexametaphosphate (HMP) for 1 hour at 48° C. before the temperature was lowered to 37° C., and template and polymerase added. The reaction was further incubated for 2 hours at 37° C. As a control, the same sequence was produced by conventional in vitro transcription (IVT). Reactions were DNase-treated, RNA purified by LiCl precipitation, and RNA quality assessed by electrophoresis using a BioAnalyzer (Agilent).

### Results

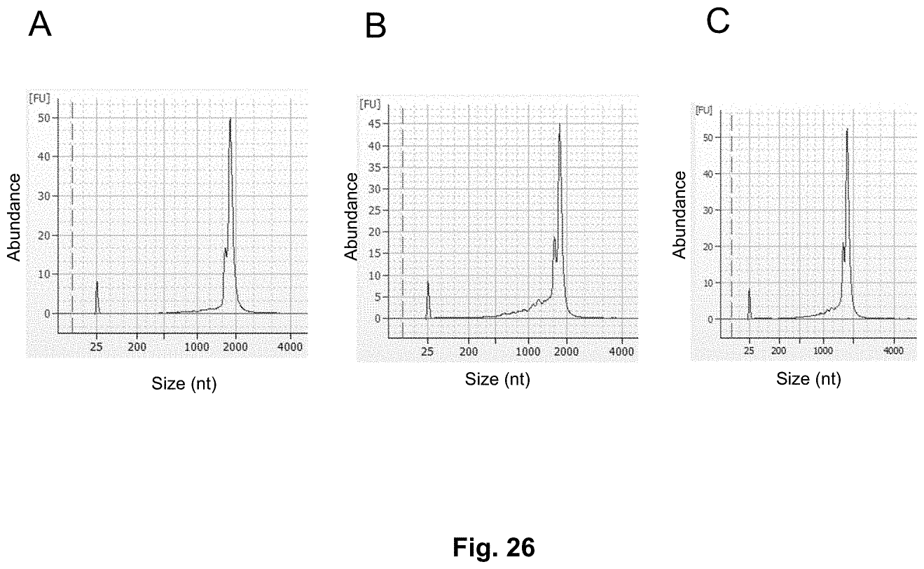

FIG. 26A is an electropherogram of uncapped IVT-produced mRNA as a reference for purity. FIG. 26B is an electropherogram of uncapped CFR-produced mRNA using cellular RNA-derived nucleotides. FIG. 26C is an electropherogram of uncapped CFR-produced mRNA using an equimolar mix of purified nucleoside monophosphates (AMP, CMP, GMP, and UMP) at 5 mM each. CFR reactions were performed similarly to those in FIG. 26B. mRNAs produced by CFR exhibited similar purity, regardless of nucleotide source, to IVT.

### Example 13: Production of mRNA with Encoded polyA Tails from Linearized Plasmid Templates

Uncapped mRNAs encoding EGFP were produced by CFR as described elsewhere in the application. Minimized template plasmids were constructed consisting of a pUC origin of replication, selectable marker, T7 promoter, EGFP gene flanked by 5′ and 3′ HBG UTRs, 3′ polyA tail, and a unique BspQI site for linearization. PolyA tails consisting of 0, 50, 100, or 150 A nucleotides were encoded in the template plasmid. Plasmids were cultivated in E. coli strain DH10b, purified by Plasmid Midi kit (Qiagen), linearized by digestion with BspQI (New England Biolabs), and purified by phenol/chloroform extraction before use in CFRs. mRNAs were synthesized, purified by lithium chloride precipitation, and analyzed by electrophoresis using a BioAnalyzer (Agilent).

### Results

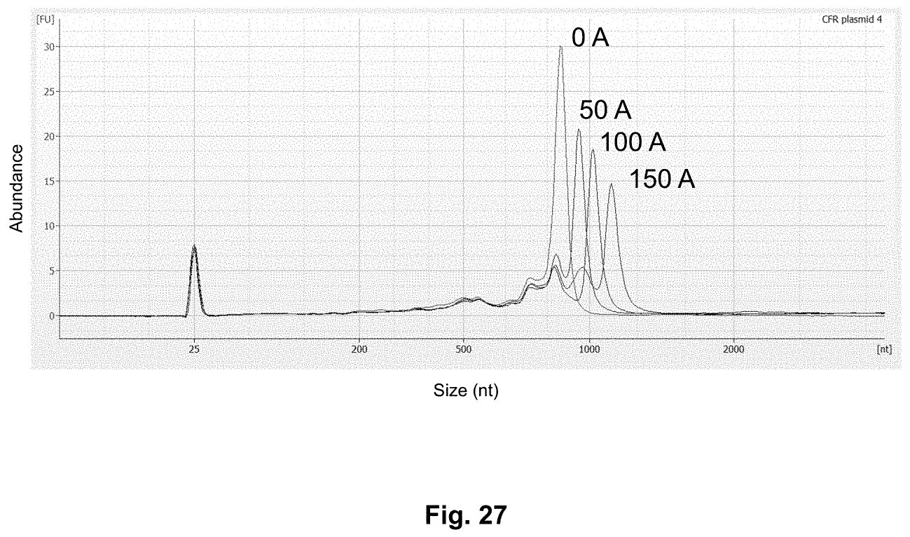

FIG. 27 is an overlay electropherogram of CFR-produced mRNAs with polyA tails of 0, 50, 100, or 150 nucleotides in length. The major peak in each sample represents a full-length mRNA of the desired size, demonstrating that the CFR system is compatible with plasmid templates and encoded polyA tails.

Having described the invention in detail and by reference to specific aspects and/or embodiments thereof, it will be apparent that modifications and variations are possible without departing from the scope of the invention defined in the appended claims. More specifically, although some aspects of the present invention can be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art can be explicitly excluded from any one or more of the claims. Because such embodiments are deemed to be known to one of ordinary skill in the art, they can be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

Sequences
Deinococcus geothermalis DSM 11300 PPK2
(SEQ ID NO: 1)
MQLDRYRVPPGQRVRLSNWPTDDDGGLSKAEGEALLPDLQQRLANLQERLYAESQQA
LLIVLQARDAGGKDGTVKHVIGAFNPSGVQVSNFKVPTEEERAHDFLWRIHRQTPRLG
MIGVFNRSQYEDVLVTRVHHLIDDQTAQRRLKHICAFESLLTDSGTRIVKFYLHISPEEQ
KKRLEARLADPSKHWKFNPGDLQERAHWDAYTAVYEDVLTTSTPAAPWYVVPADRK
WFRNLLVSQILVQTLEEMNPQFPAPAFNAADLRIV
Meiothermus ruber DM 1279 PPK2
(SEQ ID NO: 2)
MGFCSIEFLMGAQMKKYRVQPDGRFELKRFDPDDTSAFEGGKQAALEALAVLNRRLEK
LQELLYAEGQHKVLVVLQAMDAGGKDGTIRVVFDGVNPSGVRVASFGVPTEQELARD
YLWRVHQQVPRKGELVIFNRSHYEDVLVVRVKNLVPQQVWQKRYRHIREFERMLADE
GTTILKFFLHISKDEQRQRLQERLDNPEKRWKFRMGDLEDRRLWDRYQEAYEAAIRETS
TEYAPWYVIPANKNWYRNWLVSHILVETLEGLAMQYPQPETASEKIVIE
Meiothermus silvanus DSM 9946 PPK2
(SEQ ID NO: 3)
MAKTIGATLNLQDIDPRSTPGFNGDKEKALALLEKLTARLDELQEQLYAEHQHRVLVIL
QGMDTSGKDGTIRHVFKNVDPLGVRVVAFKAPTPPELERDYLWRVHQHVPANGELVIF
NRSHYEDVLVARVHNLVPPAIWSRRYDHINAFEKMLVDEGTTVLKFFLHISKEEQKKRL
LERLVEADKHWKFDPQDLVERGYWEDYMEAYQDVLDKTHTQYAPWHVIPADRKWYR
NLQVSRLLVEALEGLRMKYPRPKLNIPRLKSELEKM
Thermosynechococcus elongatus BP-1 PPK2
(SEQ ID NO: 4)
MIPQDFLDEINPDRYIVPAGGNFHWKDYDPGDTAGLKSKVEAQELLAAGIKKLAAYQD
VLYAQNIYGLLIIFQAMDAAGKDSTIKHVMSGLNPQACRVYSFKAPSAEELDHDFLWRA
NRALPERGCIGIFNRSYYEEVLVVRVHPDLLNRQQLPPETKTKHIWKERFEDINHYERYL
TRNGILILKFFLHISKAEQKKRFLERISRPEKNWKFSIEDVRDRAHWDDYQQAYADVFRH
TSTKWAPWHIIPANHKWFARLMVAHFIYQKLASLNLHYPMLSEAHREQLLEAKALLEN
EPDED
Anaerolinea thermophila UNI-1 PPK2
(SEQ ID NO: 5)
MGEAMERYFIKPGEKVRLKDWSPDPPKDFEGDKESTRAAVAELNRKLEVLQERLYAER
KHKVLVILQGMDTSGKDGVIRSVFEGVNPQGVKVANFKVPTQEELDHDYLWRVHKVV
PGKGEIVIFNRSHYEDVLVVRVHNLVPPEVWKKRYEQINQFERLLHETGTTILKFFLFISR
EEQKQRLLERLADPAKHWKFNPGDLKERALWEEYEKAYEDVLSRTSTEYAPWILVPAD
KKWYRDWVISRVLVETLEGLEIQLPPPLADAETYRRQLLEEDAPESR
Caldilinea aerophila DSM 14535 PPK2
(SEQ ID NO: 6)
MDVDRYRVPPGSTIHLSQWPPDDRSLYEGDKKQGKQDLSALNRRLETLQELLYAEGKH
KVLIILQGMDTSGKDGVIRHVFNGVNPQGVKVASFKVPTAVELAHDFLWRIHRQTPGSG
EIVIFNRSHYEDVLVVRVHGLVPPEVWARRYEHINAFEKLLVDEGTTILKFFLHISKEEQR
QRLLERLEMPEKRWKFSVGDLAERKRWDEYMAAYEAVLSKTSTEYAPWYIVPSDRKW
YRNLVISHVIINALEGLNMRYPQPEDIAFDTIVIE
Chlorobaculum tepidum TLS PPK2
(SEQ ID NO: 7)
MKLDLDAFRIQPGKKPNLAKRPTRIDPVYRSKGEYHELLANHVAELSKLQNVLYADNR
YAILLIFQAMDAAGKDSAIKHVMSGVNPQGCQVYSFKEIPSATELEHDFLWRTNCVLPE
RGRIGIFNRSYYEEVLVVRVHPEILEMQNIPHNLAHNGKVWDHRYRSIVSHEQHLHCNG
TRIVKFYLHLSKEEQRKRFLERIDDPNKNWKFSTADLEERKFWDQYMEAYESCLQETST
KDSPWFAVPADDKKNARLIVSRIVLDTLESLNLKYPEPSPERRKELLDIRKRLENPENGK
Oceanithermus profundus DSM 14977 PPK2
(SEQ ID NO: 8)
MDVSRYRVPPGSGFDPEAWPTREDDDFAGGKKEAKKELARLAVRLGELQARLYAEGR
QALLIVLQGMDTAGKDGTIRHVFRAVNPQGVRVTSFKKPTALELAHDYLWRVHRHAPA
RGEIGIFNRSHYEDVLVVRVHELVPPEVWGRRYDHINAFERLLADEGTRIVKFFLHISKD
EQKRRLEARLENPRKHWKFNPADLSERARWGDYAAAYAEALSRTSSDRAPWYAVPAD
RKWQRNRIVAQVLVDALEAMDPRFPRVDFDPASVRVE
Roseiflexus castenholzii DSM 13941 PPK2
(SEQ ID NO: 9)
MYAQRVVPGMRVRLHDIDPDANGGLNKDEGRARFAELNAELDVMQEELYAAGIHALL
LILQGMDTAGKDGAIRNVMLNLNPQGCRVESFKVPTEEELAHDFLWRVHRVVPRKGM
VGVFNRSHYEDVLVVRVHSLVPESVWRARYDQINAFERLLADTGTIIVKCFLHISKEEQE
QRLLARERDVSKAWKLSAGDWRERAFWDDYMAAYEEALTRCSTDYAPWYIIPANRK
WYRDLAISEALVETLRPYRDDWRRALDAMSRARRAELEAFRAEQHAMEGRPQGAGGV
SRR
Roseiflexus sp. RS-1 PPK2
(SEQ ID NO: 10)
MHYAHTVIPGTQVRLRDIDPDASGGLTKDEGRERFASFNATLDAMQEELYAAGVHALL
LILQGMDTAGKDGAIRNVMHNLNPQGCRVESFKVPTEEELAHDFLWRVHKVVPRKGM
VGVFNRSHYEDVLVVRVHSLVPEHVWRARYDQINAFERLLTDTGTIIVKCFLHISKDEQ
EKRLLAREQDVTKAWKLSAGDWRERERWDEYMAAYEEALTRCSTEYAPWYIIPANRK
WYRDLAISEVLVETLRPYRDDWQRALDAMSQARLAELKAFRHQQTAGATRL
Truepera radiovictrix DSM 17093 PPK2
(SEQ ID NO: 11)
MSQGSAKGLGKLDKKVYARELALLQLELVKLQGWIKAQGLKVVVLFEGRDAAGKGST
ITRITQPLNPRVCRVVALGAPTERERTQWYFQRYVHHLPAAGEMVLFDRSWYNRAGVE
RVMGFCTEAEYREFLHACPTFERLLLDAGIILIKYWFSVSAAEQERRIVIRRRNENPAKRW
KLSPMDLEARARWVAYSKAKDAMFYHTDTKASPWYVVNAEDKRRAHLSCIAHLLSLI
PYEDLTPPPLEMPPRDLAGADEGYERPDKAHQTWVPDYVPPTR
Thermus thermophilus Adk
(SEQ ID NO: 12)
MDVGQAVIFLGPPGAGKGTQASRLAQELGFKKLSTGDILRDHVARGTPLGERVRPIMER
GDLVPDDLILELIREELAERVIFDGFPRTLAQAEALDRLLSETGTRLLGVVLVEVPEEELV
RRILRRAELEGRSDDNEETVRRRLEVYREKTEPLVGYYEARGVLKRVDGLGTPDEVYA
RIRAALGI
Thermus thermophilus Cmk
(SEQ ID NO: 13)
MRGIVTIDGPSASGKSSVARRVAAALGVPYLSSGLLYRAAAFLALRAGVDPGDEEGLLA
LLEGLGVRLLAQAEGNRVLADGEDLTSFLHTPEVDRVVSAVARLPGVRAWVNRRLKEV
PPPFVAEGRDMGTAVFPEAAHKFYLTASPEVRAWRRARERPQAYEEVLRDLLRRDERD
KAQSAPAPDALVLDTGGMTLDEVVAWVLAHIRR
Pyrococcus furiosus PyrH
(SEQ ID NO: 14)
MRIVFDIGGSVLVPENPDIDFIKEIAYQLTKVSEDHEVAVVVGGGKLARKYIEVAEKFNS
SETFKDFIGIQITRANAMLLIAALREKAYPVVVEDFWEAWKAVQLKKIPVMGGTHPGHT
TDAVAALLAEFLKADLLVVITNVDGVYTADPKKDPTAKKIKKMKPEELLEIVGKGIEKA
GSSSVIDPLAAKIIARSGIKTIVIGKEDAKDLFRVIKGDHNGTTIEP
Thermotoga maritima Gmk
(SEQ ID NO: 15)
MKGQLFVICGPSGAGKTSIIKEVLKRLDNVVFSVSCTTRPKRPHEEDGKDYFFITEEEFLK
RVERGEFLEWARVHGHLYGTLRSFVESHINEGKDVVLDIDVQGALSVKKKYSNTVFIYV
APPSYADLRERILKRGTEKEADVLVRLENAKWELMFMDEFDYIVVNENLEDAVEMVVS
IVRSERAKVTRNQDKIERFKMEVKGWKKL
Aquifex aeolicus Ndk
(SEQ ID NO: 16)
MAVERTLIIVKPDAMEKGALGKILDRFIQEGFQIKALKMFRFTPEKAGEFYYVHRERPFF
QELVEFMSSGPVVAAVLEGEDAIKRVREIIGPTDSEEARKVAPNSIRAQFGTDKGKNAIH
ASDSPESAQYEICFIFSGLEIV
(SEQ ID NO: 17)
ITSGGGAGACCAGGAATT

### REFERENCES

1. Maekewa K., Tsunasawa S., Dibo G., Sakiyama F. 1991. Primary structure of nuclease P1 from Penicillium citrinum. Eur. J. Biochem. 200:651-661.
2. Volbeda A., Lahm A., Sakiyama F., Suck D. 1991. Crystal structure of Penicillium citrinum P1 nuclease at 2.8-A resolution. EMBO J. 10:1607-1618(1991)
3. Romier C., Dominguez R., Lahm A., Dahl O., Suck D. 1998. Recognition of single-stranded DNA by nuclease P1: high resolution crystal structures of complexes with substrate analogs. Proteins 32:414-424
4. Cheng Z. F., Deutscher M. P. 2002. Purification and characterization of the Escherichia coli exoribonuclease RNase R. Comparison with RNase II. J. Biol. Chem. 277:21624-21629.
5. Zilhao R., Camelo L., Arraiano C. M. 1993. DNA sequencing and expression of the gene rnb encoding Escherichia coli ribonuclease II. Mol. Microbiol. 8:43-51
6. March P. E., Ahnn J., Inouye M. 1985. The DNA sequence of the gene (rnc) encoding ribonuclease III of Escherichia coli. Nucleic Acids Res. 13:4677-4685
7. Chen S. M., Takiff H. E., Barber A. M., Dubois G. C., Bardwell J. C., Court D. L. 1990. Expression and characterization of RNase III and Era proteins. Products of the rnc operon of Escherichia coli. J. Biol. Chem. 265:2888-2895
8. Robertson H. D., Webster R. E., Zinder N. D. 1968. Purification and properties of ribonuclease III from Escherichia coli. J. Biol. Chem. 243:82-91.
9. Molina L., Bernal P., Udaondo Z., Segura A., Ramos J. L.2013. Complete Genome Sequence of a Pseudomonas putida Clinical Isolate, Strain H8234. Genome Announc. 1:E00496-13; and Cheng, Z. F. and M. P. Deutscher. 2002. Purification and characterization of the Escherichia coli exoribonuclease RNAse R. Comparison with RNAse II. J Biol Chem. 277(24).
10. Even S., Pellegrini O., Zig L., Labas V., Vinh J., Brechemmier-Baey D., Putzer H. 2005. Ribonucleases J1 and J2: two novel endoribonucleases in B. subtilis with functional homology to E. coli RNase E. Nucleic Acids Res. 33:2141-2152.
11. Li de la Sierra-Gallay I., Zig L., Jamalli A., Putzer H. 2008. Structural insights into the dual activity of RNase J. Nat. Struct. Mol. Biol. 15:206-212.
12. Ball T. K., Saurugger P. N., Benedick M. J. 1987. The extracellular nuclease gene of Serratia marcescens and its secretion from Escherichia coli. Gene 57:183-192.
13. Biedermann K., Jepsen P. K., Riise E., Svendsen I. 1989. Purification and characterization of a Serratia marcescens nuclease produced by Escherichia coli. Carlsberg Res. Commun. 54:17-27.
14. Shlyapnikov S. V., Lunin V. V., Perbandt M., Polyakov K. M., Lunin V. Y., Levdikov V. M., Betzel C., Mikhailov A. M. 2000. Atomic structure of the Serratia marcescens endonuclease at 1.1 A resolution and the enzyme reaction mechanism. Acta Crystallogr. D 56:567-572.
15. Zuo Y., Deutscher M. P. 2002. Mechanism of action of RNase T. I. Identification of residues required for catalysis, substrate binding, and dimerization. J. Biol. Chem. 277:50155-50159.
16. Zuo Y., Zheng H., Wang Y., Chruszcz M., Cymborowski M., Skarina T., Savchenko A., Malhotra A., Minor W. 2007. Crystal structure of RNase T, an exoribonuclease involved in tRNA maturation and end turnover. Structure 15:417-428.
17. Huang S., Deutscher M. P. 1992. Sequence and transcriptional analysis of the Escherichia coli rnt gene encoding RNase T. J. Biol. Chem. 267:25609-25613.
18. Chauhan A. K., Miczak A., Taraseviciene L., Apirion D. 1991. Sequencing and expression of the me gene of Escherichia coli. Nucleic Acids Res. 19:125-129.
19. Cormack R. S., Genereaux J. L., Mackie G. A. 1993. RNase E activity is conferred by a single polypeptide: overexpression, purification, and properties of the ams/rne/hmp1 gene product. Proc. Natl. Acad. Sci. U.S.A. 90:9006-9010.
20. Motomura, K., Hirota, R., Okada, M., Ikeda, T., Ishida, T., & Kuroda, A. (2014). A New Subfamily of Polyphosphate Kinase 2 (Class III PPK2) Catalyzes both Nucleoside Monophosphate Phosphorylation and Nucleoside Diphosphate Phosphorylation. Applied and Environmental Microbiology, 80(8), 2602-2608. http://doi.org/10.1128/AEM.03971-13
21. Elkin, S. R., Kumar, A., Price, C. W., & Columbus, L. (2013). A Broad Specificity Nucleoside Kinase from Thermoplasma acidophilum. Proteins, 81(4), 568-582. doi.org/10.1002/prot.24212
22. Hansen, T., Arnfors, L., Ladenstein, R., & Schonheit, P. (2007). The phosphofructokinase-B (MJ0406) from Methanocaldococcus jannaschii represents a nucleoside kinase with a broad substrate specificity. Extremophiles, 11(1), 105.
23. Ota, H., Sakasegawa, S., Yasuda, Y., Imamura, S., & Tamura, T. (2008). A novel nucleoside kinase from Burkholderia thailandensis: a member of the phosphofructokinase B-type family of enzymes. The FEBS journal, 275(23), 5865.
24. Tomoike, F., Nakagawa, N., Kuramitsu, S., & Masui, R. (2011). A single amino acid limits the substrate specificity of Thermus thermophilus uridine-cytidine kinase to cytidine. Biochemistry, 50(21), 4597.
25. Henne A., Brueggemann H., Raasch C., Wiezer A., Hartsch T., Liesegang H., Johann A., Lienard T., Gohl O., Martinez-Arias R., Jacobi C., Starkuviene V., Schlenczeck S., Dencker S., Huber R., Klenk H.-P., Kramer W., Merkl R., Gottschalk G., Fritz H.-J. 2004. The genome sequence of the extreme thermophile Thermus thermophilus. Nat. Biotechnol. 22:547-553.
26. Tan Z W, Liu J, Zhang X F, Meng F G, Zhang Y Z. Nan Fang Yi Ke Da Xue Xue Bao. 2010. Expression, purification and enzymatic characterization of adenylate kinase of Thermus thermophilus HB27 in Escherichia coli. January; 30(1):1-6
27. Maeder D. L., Weiss R. B., Dunn D. M., Cherry J. L., Gonzalez J. M., DiRuggiero J., Robb F. T. 1999. Divergence of the hyperthermophilic archaea Pyrococcus furiosus and P. horikoshii inferred from complete genomic sequences. Genetics 152:1299-1305.
28. Methe, B. A., Nelson, K. E., Deming, J. W., Momen, B., Melamud, E., Zhang, X., . . . Fraser, C. M. (2005). The psychrophilic lifestyle as revealed by the genome sequence of Colwellia psychrerythraea 34H through genomic and proteomic analyses. Proceedings of the National Academy of Sciences of the United States of America, 102(31), 10913-10918. doi.org/10.1073/pnas.0504766102
29. Medigue, C., Krin, E., Pascal, G., Barbe, V., Bernsel, A., Bertin, P. N., . . . Danchin, A. (2005). Coping with cold: The genome of the versatile marine Antarctica bacterium Pseudoalteromonas haloplanktis TAC125. Genome Research, 15(10), 1325-1335. doi.org/10.1101/gr.4126905
30. Ayala-del-Rio, H. L., Chain, P. S., Grzymski, J. J., Ponder, M. A., Ivanova, N., Bergholz, P. W., . . . Tiedje, J. M. (2010). The Genome Sequence of Psychrobacter arcticus 273-4, a Psychroactive Siberian Permafrost Bacterium, Reveals Mechanisms for Adaptation to Low-Temperature Growth. Applied and Environmental Microbiology, 76(7), 2304-2312. doi.org/10.1128/AEM.02101-09
31. Feil, H., Feil, W. S., Chain, P., Larimer, F., DiBartolo, G., Copeland, A., . . . Lindow, S. E. (2005). Comparison of the complete genome sequences of Pseudomonas syringae pv. syringae B728a and pv. tomato DC3000. Proceedings of the National Academy of Sciences of the United States of America, 102(31), 11064-11069. doi.org/10.1073/pnas.0504930102
32. Song, S., Inouye, S., Kawai, M., Fukami-Kobayashi, K., Gō, M., & Nakazawa, A. (1996). Cloning and characterization of the gene encoding Halobacterium halobium adenylate kinase. Gene, 175(1), 65-70.
33. Masui R., Kurokawa K., Nakagawa N., Tokunaga F., Koyama Y., Shibata T., Oshima T., Yokoyama S., Yasunaga T., Kuramitsu S. Complete genome sequence of Thermus thermophilus HB8. Submitted (November 2004) to the EMBL/GenBank/DDBJ databases.
34. Ng, W. V., Kennedy, S. P., Mahairas, G. G., Berquist, B., Pan, M., Shukla, H. D., . . . DasSarma, S. (2000). Genome sequence of Halobacterium species NRC-1. Proceedings of the National Academy of Sciences of the United States of America, 97(22), 12176-12181.
35. Marco-Marin C., Escamilla-Honrubia J. M., Rubio V. 2005. First-time crystallization and preliminary X-ray crystallographic analysis of a bacterial-archaeal type UMP kinase, a key enzyme in microbial pyrimidine biosynthesis. Biochim. Biophys. Acta 1747:271-275.
36. Marco-Marin C., Escamilla-Honrubia J. M., Rubio V. 2005. First-time crystallization and preliminary X-ray crystallographic analysis of a bacterial-archaeal type UMP kinase, a key enzyme in microbial pyrimidine biosynthesis. Biochim. Biophys. Acta 1747:271-275.
37. Jensen, K. S., Johansson, E., & Jensen, K. F. (2007). Structural and enzymatic investigation of the Sulfolobus solfataricus uridylate kinase shows competitive UTP inhibition and the lack of GTP stimulation. Biochemistry, 46(10), 2745-2757.
38. Nelson K. E., Clayton R. A., Gill S. R., Gwinn M. L., Dodson R. J., Haft D. H., Hickey E. K., Peterson J. D., Nelson W. C., Ketchum K. A., McDonald L. A., Utterback T. R., Malek J. A., Linher K. D., Garrett M. M., Stewart A. M., Cotton M. D., Pratt M. S. Fraser C. M. 1999. Evidence for lateral gene transfer between Archaea and Bacteria from genome sequence of Thermotoga maritima. Nature 399:323-329.
39. Riley, M., Staley, J. T., Danchin, A., Wang, T. Z., Brettin, T. S., Hauser, L. J., . . . Thompson, L. S. (2008). Genomics of an extreme psychrophile, Psychromonas ingrahamii. BMC Genomics, 9,210. doi.org/10.1186/1471-2164-9-210
40. Ishibashi, M., Tokunaga, H., Hiratsuka, K., Yonezawa, Y., Tsurumaru, H., Arakawa, T., & Tokunaga, M. (2001). NaCl-activated nucleoside diphosphate kinase from extremely halophilic archaeon, Halobacterium salinarum, maintains native conformation without salt. FEBS letters, 493(2-3), 134.
41. Polosina, Y. Y., Zamyatkin, D. F., Kostyukova, A. S., Filimonov, V. V., & Fedorov, O. V. (2002). Stability of Natrialba magadii NDP kinase: comparisons with other halophilic proteins. Extremophiles: life under extreme conditions, 6(2), 135.
42. Polosina, Y. Y., Zamyatkin, D. F., Kostyukova, A. S., Filimonov, V. V., & Fedorov, O. V. (2002). Stability of Natrialba magadii NDP kinase: comparisons with other halophilic proteins. Extremophiles: life under extreme conditions, 6(2), 135.
43. Udaondo, Z., Molina, L., Daniels, C., Gómez, M. J., Molina-Henares, M. A., Matilla, M. A., . . . Ramos, J. L. (2013). Metabolic potential of the organic-solvent tolerant Pseudomonas putida DOT-T1E deduced from its annotated genome. Microbial Biotechnology, 6(5), 598-611. http://doi.org/10.1111/1751-7915.12061
44. Nölling, J., Breton, G., Omelchenko, M. V., Makarova, K. S., Zeng, Q., Gibson, R., Smith, D. R. (2001). Genome Sequence and Comparative Analysis of the Solvent-Producing Bacterium Clostridium acetobutylicum. Journal of Bacteriology, 183(16), 4823-4838. http://doi.org/10.1128/JB.183.16.4823-4838.2001
45. Brune, M., Schumann, R., & Wittinghofer, F. (1985). Cloning and sequencing of the adenylate kinase gene (adk) of Escherichia coli. Nucleic Acids Research, 13(19), 7139-7151.
46. Pel, H. J., de Winde, J. H., Archer, D. B., Dyer, P. S., Hofmann, G., Schaap, P. J., . . . & Andersen, M. R. (2007). Genome sequencing and analysis of the versatile cell factory Aspergillus niger CBS 513.88. Nature biotechnology, 25(2), 221.
47. Magdolen, V., Oechsner, U., & Bandlow, W. (1987). The complete nucleotide sequence of the gene coding for yeast adenylate kinase. Current genetics, 12(6), 405.
48. Pedersen, S., Skouv, J., Kajitani, M., & Ishihama, A. (1984). Transcriptional organization of the rpsA operon of Escherichia coli. Molecular & general genetics: MGG, 196(1), 135.
49. Smallshaw, J., & Kelln, R. A. (1992). Cloning, nucleotide sequence and expression of the Escherichia coli K-12 pyrH gene encoding UMP kinase. Genetics (Life Sci. Adv.), 11, 59-65.
50. Liljelund, P., Sanni, A., Friesen, J. D., & Lacroute, F. (1989). Primary structure of the S. cerevisiae gene encoding uridine monophosphokinase. Biochemical and biophysical research communications, 165(1), 464.
51. Gentry, D., Bengra, C., Ikehara, K., & Cashel, M. (1993). Guanylate kinase of Escherichia coli K-12. The Journal of biological chemistry, 268(19), 14316.
52. Konrad, M. (1992). Cloning and expression of the essential gene for guanylate kinase from yeast. The Journal of biological chemistry, 267(36), 25652.
53. Hama, H., Almaula, N., Lerner, C. G., Inouye, S., & Inouye, M. (1991). Nucleoside diphosphate kinase from Escherichia coli; its overproduction and sequence comparison with eukaryotic enzymes. Gene, 105(1), 31.
54. Besir, H., Zeth, K., Bracher, A., Heider, U., Ishibashi, M., Tokunaga, M., & Oesterhelt, D. (2005). Structure of a halophilic nucleoside diphosphate kinase from Halobacterium salinarum. FEBS letters, 579(29), 6595.
55. Deutscher, M. & Reuven N. (1991). Enzymatic basis for hydrolytic versus phosphorolytic mRNA degradation in Escherichia coli and Bacillus subtilis. PNAS, 88, 3277-3280.
56. Nwokeji, A. O., Kilby, P. M., Portwood, D. E., & Dickman, M. J. (2016). RNASwift: A rapid, versatile RNA extraction method free from phenol and chloroform. Analytical Biochemistry, 512, 36-46.
57. Mohanty, B. K., Giladi, H., Maples, V. F., & Kushner, S. R. (2008). Analysis of RNA decay, processing, and polyadenylation in Escherichia coli and other prokaryotes. Methods in Enzymology, 447, 3-29.
58. Korz, D. J., Rinas, U., Hellmuth, K., Sanders, E. A., & Deckwer, W. D. (1995). Simple fed-batch technique for high cell density cultivation of Escherichia coli. Journal of biotechnology, 39(1), 59-654
59. Phue, J. N., Lee, S. J., Trinh, L., & Shiloach, J. (2008). Modified Escherichia coli B (BL21), a superior producer of plasmid DNA compared with Escherichia coli K (DH5alpha). Biotechnology and bioengineering, 101(4), 831.
60. de Korte, D., Haverkort, W. A., Roos, D., & van Gennip, A. H. (1985). Anion-exchange high performance liquid chromatography method for the quantitation of nucleotides in human blood cells. Clinica chimica acta; international journal of clinical chemistry, 148(3), 185.]

1. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; and
(b) treating said reaction mixture under conditions wherein the RNA depolymerizing enzymes are eliminated or inactivated; and
(c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, v) at least one nucleoside-diphosphate (NDP) kinase, under conditions wherein nucleotide triphosphates are produced and further wherein vi) at least one RNA polymerase, vii) at least one DNA template encoding an mRNA with a polyA tail, viii) one or more capping reagents are added under conditions that produce mRNA, and further wherein, optionally ix) at least one deoxyribonuclease is added under conditions that digest the DNA following RNA production.

2. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; and
(b) treating said reaction mixture under conditions wherein the RNA depolymerizing enzymes are eliminated or inactivated; and
(c) incubating said reaction mixture comprising nucleoside monophosphates with a second reaction mixture comprising i) at least one polyphosphate (PPK) kinase and a phosphate donor and optionally ii) at least one cytidine monophosphate (CMP) kinase, iii) at least one uridine monophosphate (UMP) kinase, iv) at least one guanosine monophosphate (GMP) kinase, v) at least one nucleoside-diphosphate (NDP) kinase, under conditions wherein nucleotide triphosphates are produced and further wherein vi) at least one RNA polymerase, vii) at least one DNA template encoding an mRNA, and viii) one or more capping reagents are added under conditions that produce capped RNA; and further wherein optionally ix) at least one deoxyribonuclease is added under conditions that digest the DNA following RNA production; and
d) (i) further incubating said reaction mixture produced in step (c) in the presence of a polyA polymerase and ATP, under conditions that produce mRNA
or
(ii) removing the RNA polymerase from the reaction mixture of step (c) by inactivation or physical separation followed by producing mRNA by further incubating the reaction mixture in the presence of polyA polymerase and ATP.

3. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; and
(b) treating said reaction mixture under conditions wherein the RNA depolymerizing enzymes are eliminated or inactivated; and
(c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, v) at least one nucleoside-diphosphate (NDP) kinase, under conditions wherein nucleotide triphosphates are produced and further wherein vi) at least one RNA polymerase vii) at least one DNA template encoding an mRNA with a poly A tail, are added under conditions that produce uncapped RNA, and further wherein optionally viii) at least one deoxyribonuclease is added under conditions that digest the DNA following RNA production; and
(d) exchanging the buffer from said reaction mixture from step (c) and incubating said reaction mixture in the presence of capping enzymes, GTP, and a methyl donor to produce mRNA.

4. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises 5′ nucleoside monophosphates; and
(b) treating said reaction mixture under conditions wherein the RNA depolymerizing enzymes are eliminated or inactivated; and
(c) incubating said reaction mixture comprising nucleoside monophosphates with a second reaction mixture comprising i) at least one polyphosphate (PPK) kinase and a phosphate donor; and optionally ii) at least one cytidine monophosphate (CMP) kinase, iii) at least one uridine monophosphate (UMP) kinase, iv) at least one guanosine monophosphate (GMP) kinase, v) at least one nucleoside-diphosphate (NDP) kinase, under conditions wherein nucleotide triphosphates are produced and further wherein vi) at least one RNA polymerase vii) at least one DNA template encoding an mRNA, are added under conditions that produce uncapped, untailed RNA, and further wherein, optionally viii) at least one deoxyribonuclease is added under conditions that digest the DNA following RNA production; and
d) (i) further incubating said reaction mixture produced in step (c) in the presence of a polyA polymerase and ATP, under conditions that produce uncapped RNA;
or
(ii) removing the RNA polymerase from the reaction mixture of step (c) by inactivation or physical separation followed by producing uncapped RNA by further incubating the reaction mixture in the presence of polyA polymerase and ATP; and
e) exchanging the buffer from said reaction mixture from step (c) and incubating said reaction mixture in the presence of capping enzymes, GTP, and a methyl donor to produce mRNA.

5. The method of any one of claims 1-4, wherein steps (c)(i)-(c)(v) are performed to produce nucleotide triphosphates before the remaining steps of (c), instead of concurrently.

6. The method of claim 1 or 2, wherein the capping reagents are dinucleotide, trinucleotide, or tetranucleotide capping reagents.

7. The method of any one of claims 1-4, wherein the cellular RNA is derived from biomass.

8. The method of step (a) of any one of claims 1-4, wherein the enzyme that depolymerizes RNA into 5′ nucleoside monophosphates is Nuclease P1.

9. The method of any one of claims 1-4, wherein the second reaction mixture of step (c) comprises an enzyme preparation obtained from cells that produce the PPK, the NMP kinases, the NDP kinase, the deoxyribonucleic acid (DNA) template, and/or the RNA polymerase.

10. The method of any one of claims 1-4, wherein the at least one cytidine monophosphate kinase is from Thermus thermophilus.

11. The method of any one of claims 1-4, wherein the at least one uridine monophosphate kinase is from Pyroccus furiosus.

12. The method of any one of claims 1-4, wherein the at least one guanosine monophosphate kinase is from Thermatoga maritima.

13. The method of any one of claims 1-4, wherein the at least one nucleoside diphosphate kinase is from Aquifex aeolicus.

14. The method of any one of claims 1-4, wherein the at least one polyphosphate kinase is a Class III polyphosphate kinase 2 from Deinococcus geothermalis.

15. The method of any one of claims 1-4, wherein the phosphate donor is hexametaphosphate.

16. The method of any one of claims 1-4, wherein the RNA polymerase is bacteriophage T7 RNA polymerase or mutants thereof.

17. The method of any one of claims 1-4, wherein the DNA template comprises: i) sequence encoding an open reading frame (ORF) for the resulting mRNA; and/or ii) a transcriptional promoter, iii) sequence encoding a 5′ untranslated region (5′ UTR) for the resulting mRNA, iv) sequence encoding a 3′ untranslated region (3′ UTR) for the resulting mRNA and optionally, a recognition site for a restriction endonuclease.

18. The method of claim 17, wherein the DNA template further comprises a sequence encoding one or more internal ribosome entry site (IRES) elements.

19. The method of claim 17 or 18, wherein the DNA template is produced by polymerase chain reaction.

20. The method of claim 17 or 18, wherein the DNA template is encoded on a plasmid.

21. The method of claim 20, wherein the plasmid contains one or a plurality of DNA templates encoding one or a plurality of mRNAs.

22. The method of claim 20 or 21, wherein the plasmid DNA is linearized using a restriction endonuclease.

23. The method of claim 22, wherein the plasmid DNA is linearized using a type IIS restriction endonuclease.

24. The method of claim 22 or 23, wherein the plasmid DNA and/or restriction endonuclease are purified before or after linearization.

25. The method of claim 2 or 4, wherein the poly(A) polymerase is thermostable.

26. The method of claim 3 or 4, wherein the one or more capping enzymes of step 3d or 4e have phosphatase activity, methyltransferase activity, and/or guanylyltransferase activity.

27. The method of claim 3 or 4, wherein the methyl donor is S-Adenosyl methionine.

28. The method of claim 3 or 4, wherein the one or more capping enzymes is derived from Vaccinia virus and/or Blue Tongue virus.

29. The method of claim 3 or 4, wherein the one or more capping enzymes has phosphatase activity.

30. The method of claim 3 or 4, wherein the one or more capping enzymes has methyltransferase activity.

31. The method of claim 3 or 4, wherein the one or more capping enzymes has guanylyltransferase activity.

32. The method of any one of claims 1-4, wherein the second reaction mixture, comprising PPK, optionally NMP kinases, optionally NDP kinase, deoxyribonucleic acid (DNA) template optionally comprising a restriction endonuclease recognition site, RNA polymerase, polyA polymerase, and/or capping enzymes is prepared from one or more cell lysates wherein, when present in said cell lysates, undesired enzymatic activities are eliminated, inactivated, or partially inactivated.

33. The method of claim 32, wherein the undesired enzymatic activities are eliminated by physical separation or inactivated or partially inactivated by heat treatment.

34. The method of claim 33, wherein the temperature that inactivates or partially inactivates the enzymes is between 70° C. and 95° C.

35. The method of claim 33, wherein the temperature that inactivates or partially inactivates the enzymes is equal to or greater than 55° C.

36. The method of claim 32, wherein the second reaction mixture comprises one or more enzymes purified from cell lysates by chromatography.

37. The method of claim 32, wherein the capping enzymes are separated from undesired enzymatic activities using chromatography.

38. The method of any one of claims 1-4, wherein the method further comprises purification of the mRNA by filtration, extraction, precipitation, or chromatography.

39. The method of claim 38, wherein the mRNA is further purified by lithium chloride precipitation.

40. The method of claim 38 or 39 wherein the mRNA is further purified by high-performance liquid chromatography.

41. An mRNA produced by the method of any one of claims 1-40.

42. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises nucleoside diphosphates;
(b) treating said reaction mixture under conditions wherein the RNA depolymerizing enzymes are eliminated or inactivated; and
(c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, v) at least one guanosine monophosphate (GMP) kinase, under conditions wherein nucleotide triphosphates are produced and further wherein vi) at least one RNA polymerase, vii) at least one DNA template encoding an mRNA with a polyA tail, viii) one or more capping reagents are added under conditions that produce mRNA, and further wherein optionally ix) at least one deoxyribonuclease is added under conditions that digest the DNA following RNA production.

43. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises nucleoside diphosphates; and
(b) treating said reaction mixture under conditions wherein the RNA depolymerizing enzymes are eliminated or inactivated; and
(c) incubating said reaction mixture comprising nucleoside diphosphates with a second reaction mixture comprising i) at least one polyphosphate (PPK) kinase and a phosphate donor and optionally ii) at least one nucleoside-diphosphate (NDP) kinase, and optionally iii) at least one cytidine monophosphate (CMP) kinase, iv) at least one uridine monophosphate (UMP) kinase, v) at least one guanosine monophosphate (GMP) kinase, under conditions wherein nucleotide triphosphates are produced and further wherein vi) at least one RNA polymerase vii) at least one DNA template encoding an mRNA; viii) one or more capping reagents are added under conditions that produce capped RNA, and further wherein optionally ix) at least one deoxyribonuclease is added under conditions that digest the DNA following RNA production; and
d) (i) further incubating said reaction mixture produced in step (c) in the presence of a polyA polymerase and ATP, under conditions that produce mRNA
or
(ii) removing the RNA polymerase from the reaction mixture of step (c) by inactivation or physical separation followed by producing mRNA by further incubating the reaction mixture in the presence of polyA polymerase and ATP.

44. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises nucleoside diphosphates;
(b) treating said reaction mixture under conditions wherein the RNA depolymerizing enzymes are eliminated or inactivated;
(c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, v) at least one guanosine monophosphate (GMP) kinase, under conditions wherein nucleotide triphosphates are produced and further wherein vi) at least one RNA polymerase and vii) at least one DNA template encoding an mRNA with a poly A tail, are added under conditions that produce uncapped RNA; and further wherein optionally viii) at least one deoxyribonuclease is added under conditions that digest the DNA following RNA production; and
(d) exchanging the buffer from said reaction mixture from step (c) and incubating said reaction mixture in the presence of capping enzymes, GTP, and a methyl donor to produce mRNA.

45. A cell-free reaction method for synthesizing a eukaryotic messenger ribonucleic acid (mRNA), the method comprising:
(a) incubating in a reaction mixture cellular RNA and one or more enzymes that depolymerize RNA under conditions wherein the cellular RNA is substantially depolymerized, wherein the resulting first reaction mixture comprises nucleoside diphosphates; and
(b) treating said reaction mixture under conditions wherein the RNA depolymerizing enzymes are eliminated or inactivated; and
(c) incubating said reaction mixture comprising nucleoside diphosphates with a second reaction mixture comprising i) at least one polyphosphate (PPK) kinase and a phosphate donor; and optionally ii) at least one nucleoside-diphosphate (NDP) kinase, and optionally iii) at least one cytidine monophosphate (CMP) kinase, iv) at least one uridine monophosphate (UMP) kinase, v) at least one guanosine monophosphate (GMP) kinase, vi) under conditions wherein nucleotide triphosphates are produced and further wherein vii) at least one RNA polymerase and viii) at least one DNA template encoding an mRNA, are added under conditions that produce uncapped, untailed RNA, and further wherein optionally ix) at least one deoxyribonuclease is added under conditions that digest the DNA following RNA production;
(d) (i) further incubating said reaction mixture produced in step (c) in the presence of a polyA polymerase and ATP, under conditions that produce uncapped RNA;
or
(ii) removing the RNA polymerase from the reaction mixture of step (c) by inactivation or physical separation followed by producing uncapped RNA by further incubating the reaction mixture in the presence of polyA polymerase and ATP; and
e) exchanging the buffer from said reaction mixture from step (c) and incubating said reaction mixture in the presence of capping enzymes, GTP, and a methyl donor to produce mRNA.

46. The method of any one of claims 42-45, wherein steps (c)(i)-(c)(v) are performed to produce nucleotide triphosphates before the remaining steps of (c), instead of concurrently.

47. The method of claim 42 or 43, wherein the capping reagents are dinucleotide, trinucleotide, or tetranucleotide capping reagents.

48. The method of any one of claims 42-45, wherein the cellular RNA is derived from biomass.

49. The method of any one of claims 42-45, wherein the RNA depolymerizing enzyme is a ribonuclease that creates 5′ nucleoside diphosphates (NDPs).

50. The method of any one of claims 42-45, wherein the enzyme that depolymerizes RNA into 5′ nucleoside diphosphates is PNPase.

51. The method of any one of claims 42-45, wherein the second reaction mixture of step (c) comprises an enzyme preparation obtained from cells that produce PPK, NDP kinase, NMP kinases, deoxyribonucleic acid (DNA) template, and/or the RNA polymerase.

52. The method of any one of claims 42-45, wherein the at least one cytidine monophosphate kinase is from Thermus thermophilus.

53. The method of any one of claims 42-45, wherein the at least one uridine monophosphate kinase is from Pyroccus furiosus.

54. The method of any one of claims 42-45, wherein the at least one guanosine monophosphate kinase is from Thermatoga maritima

55. The method of any one of claims 42-45, wherein the at least one nucleoside diphosphate kinase is from Aquifex aeolicus.

56. The method of any one of claims 42-45, wherein the at least one polyphosphate kinase is a Class III polyphosphate kinase 2 from Deinococcus geothermalis.

57. The method of any one of claims 42-45, wherein the phosphate donor is hexametaphosphate.

58. The method of any one of claims 42-45, wherein the RNA polymerase is bacteriophage T7 RNA polymerase or mutants thereof.

59. The method of any one of claims 42-45, wherein the DNA template comprises: i) sequence encoding an open reading frame (ORF) for the resulting mRNA; and/or ii) a transcriptional promoter, iii) sequence encoding a 5′ untranslated region (5′ UTR) for the resulting mRNA, iv) sequence encoding an open reading frame (ORF) for the resulting mRNA, v) sequence encoding a 3′ untranslated region (3′ UTR) for the resulting mRNA and/or optionally, a recognition site for a restriction endonuclease.

60. The method of any one of claim 59, wherein the DNA template further comprises a sequence encoding one or more internal ribosome entry site (IRES) elements.

61. The method of claim 59 or 60, wherein the DNA template is produced by polymerase chain reaction.

62. The method of claim 59 or 60, wherein the DNA template is encoded on a plasmid.

63. The method of claim 62, wherein the plasmid contains one or a plurality of DNA templates encoding one or a plurality of mRNAs.

64. The method of claim 62 or 63 wherein the plasmid DNA is linearized using a restriction endonuclease.

65. The method of claim 64, wherein the plasmid DNA is linearized using a type IIS restriction endonuclease.

66. The method of claim 64 or 65, wherein the plasmid DNA and/or restriction endonuclease are purified before or after linearization.

67. The method of claim 43 or 45, wherein the poly(A) polymerase is thermostable.

68. The method of claim 44 or 45, wherein the one or more capping enzymes of step 44d or 45e have phosphatase activity, methyltransferase activity, and/or guanylyltransferase activity.

69. The method of claim 44 or 45, wherein the methyl donor is S-Adenosyl methionine.

70. The method of claim 44 or 45, wherein the one or more capping enzymes is derived from Vaccinia virus and/or Blue Tongue virus.

71. The method of claim 44 or 45, wherein the one or more capping enzymes has phosphatase activity.

72. The method of claim 44 or 45, wherein the one or more capping enzymes has methyltransferase activity.

73. The method of claim 44 or 45, wherein the one or more capping enzymes has guanylyltransferase activity.

74. The method of any one of claims 42-45, wherein the second reaction mixture comprising the PPK, optionally the NDP kinase, optionally the NMP kinases, the deoxyribonucleic acid (DNA) template optionally comprising a restriction endonuclease recognition site the RNA polymerase, the polyA polymerase, and/or the capping enzymes is prepared from one or more cell lysates wherein, when present in said cell lysates, undesired enzymatic activities are eliminated, inactivated, or partially inactivated.

75. The method of claim 74, wherein the undesired enzymatic activities are eliminated by physical separation or inactivated or partially inactivated by heat treatment.

76. The method of claim 75, wherein the temperature that inactivates or partially inactivates the enzymes is between 70° C. and 95° C.

77. The method of claim 75, wherein the temperature that inactivates or partially inactivates the enzymes is equal to or greater than 55° C.

78. The method of claim 32 or 74 wherein the RNA polymerase is hexahistidine-tagged and purified by immobilized metal affinity chromatography.

79. The method of claim 74, wherein the capping enzymes are separated from undesired enzymatic activities using chromatography.

80. The method of any one of claims 42-45, wherein the method further comprises purification of the mRNA by filtration, extraction, precipitation, or chromatography.

81. The method of claim 80, wherein the mRNA is further purified by lithium chloride precipitation.

82. The method of claim 80 or 81, wherein the mRNA is further purified by high-performance liquid chromatography.

83. The method of any one of claims 38-40 or claims 80-82, wherein the mRNA is further purified using reversed-phase ion-pair high performance liquid chromatography.

84. An mRNA produced by the method of any one of claims 42-83.

85. The method of any one of claims 1-40 or 42-83, wherein nucleotides produced by means other than steps (a) and (b) are added to the reaction mixture of step (c), thereby eliminating the need for steps (a) and (b).

86. The method of claim 85, wherein the nucleotides include NMPs, NDPs, NTPs, or a mixture thereof.

87. The method of claim 85, wherein the nucleotides consist of one or more of unmodified nucleotides, modified nucleotides, or mixtures thereof.

88. The method of claim 87, wherein the nucleotides consist of one or more unmodified NMPs and one or more modified NTPs, to create mRNAs with 100% replacement of one or more unmodified nucleotides with modified nucleotides.

89. The method of claim 88, wherein the nucleotides comprise unmodified AMP, CMP, and GMP, and pseudouridine triphosphate (pseudoUTP).

90. The method of claim 88, wherein the nucleotides comprise unmodified AMP and GMP with pseudoUTP and 5-methylcytidine triphosphate (5-methyl CTP).

91. The method of claim 87, wherein the nucleotides comprise a mixture containing one or more of unmodified AMP, CMP, UMP, and GMP with pseudoUTP and/or 5-methyl CTP.

92. The method of any one of claims 1-91, wherein modified nucleotides are added to cellular-RNA-derived nucleotides to achieve partially modified mRNA.

93. The method of any one of claims 2, 4, 43, or 45 step (c) or step (d), wherein the ATP is added directly.

94. The method of any one of claims 2, 4, 43, or 45 step (c) or step (d), wherein purified AMP or ADP plus a phosphate donor in the presence of PPK is added to produce ATP.

95. The method of any one of claims 2, 4, 43, or 45 step (c) or step (d), wherein AMP or ADP derived from cellular RNA and a phosphate donor in the presence of PPK to produce ATP.

96. The method of any one of claims 3d, 4e, 44d, or 45e, wherein the GTP is added directly.

97. The method of any one of claims 3d, 4e, 44d, or 45e, wherein purified GMP or GDP plus a phosphate donor in the presence of one or more kinases to produce GTP.

98. The method of any one of claims 3d, 4e, 44d, or 45e, wherein GMP or GDP derived from cellular RNA and a phosphate donor in the presence of one or more kinases to produce GTP.

99. The method of claim 9 or 51, wherein the second reaction mixture of step (c) comprises a cell lysate obtained from cells that produce PPK, NDP kinase, NMP kinases, deoxyribonucleic acid (DNA) template, and/or the RNA polymerase.

100. The method of claim 7 or 48, wherein the biomass comprises yeast.

101. An mRNA produced by the method of any one of claims 85-100.
